(19) United States
(12) Patent Application Publication
(10) Pub. No.: US 2014/0378541 A1
(43) Pub. Date: Dec. 25, 2014

(54) Synthetic Epigallocatechin Gallate (EGCG) Analogs

(71) Applicants: Tak-Hang Chan, Toronto (CA); Sreedhar Pamu, Toronto (CA); Qing Ping Dou, Grosse Pointe, MI (US); Di Chen, Detroit, MI (US)

(72) Inventors: Tak-Hang Chan, Toronto (CA); Sreedhar Pamu, Toronto (CA); Qing Ping Dou, Grosse Pointe, MI (US); Di Chen, Detroit, MI (US)

(73) Assignees: WAYNE STATE UNIVERSITY, Detroit, MI (US); THE ROYAL INSTITUTION FOR THE ADVANCEMENT OF LEARNING/MCGILL UNIVERSITY, Montreal (CA); HONG KONG POLYTECHNIC UNIVERSITY, Kowloon, HK (CN)

(21) Appl. No.: 14/126,667

(22) PCT Filed: Jun. 15, 2012
(86) PCT No.: PCT/CA2012/000596

§ 371 Date: Mar. 31, 2014

Related U.S. Application Data
(60) Provisional application No. 61/497,582, filed on Jun. 16, 2011.

(51) Int. Cl. C07C 69/84; A61K 45/06; C07C 69/92; C12N 9/12; A61K 31/245; C07C 233/54; A61K 31/24; A61K 31/235; C07C 229/60

(52) U.S. Cl. CPC C07C 69/84; A61K 31/235; A61K 45/06; C07C 69/92; C07C 229/60; A61K 31/245; C07C 233/54; A61K 31/24; C12N 9/12; C07C 2102/10; C12Y 207/11031
USPC 514/533; 560/70; 560/65; 560/73; 560/46; 560/49; 560/107; 435/375

(57) ABSTRACT

Synthetic polyphenolic compounds of formula (I), their modes of synthesis, and pharmaceutical compositions thereof are provided herein. Use of the compounds and compositions described herein for treating cancer and for treating metabolic disorders is also provided.

### FIELD OF THE INVENTION

This invention relates to novel compounds and compositions comprising analogs of epigallocatechin gallate, particularly for use as proteasome inhibitors and/or AMPK activators and for treating cancer.

### BACKGROUND OF THE INVENTION

The ubiquitin-proteasome system (UPS) is responsible for the highly regulated degradation of intracellular proteins having important roles in cellular functions (Hershko A (2005) Cell Death Differ. 12, 1191). One compound that targets the UPS is the proteasome inhibitor bortezomib (Velcade™), which is used clinically for the treatment of patients with multiple myeloma or mantle cell lymphoma. Velcade™ is an N-substituted dipeptidyl boronic acid. Another proteasome inhibitor is salinosporamide, a marine natural product characterized by a functionalized β lactone (Feling R H et al. (2003) Angew. Chem. Int. Ed. Engl. 42, 355). Yet another inhibitor of the proteasome is epigallocatechin gallate (EGCG) and its analogs (U.S. Pat. No. 7,358,383 B2; U.S. Pat. No. 6,713,506 B2; US 2008/0015248 A1; WO 2006/017981; Landis-Piwowar K R et al. (2007) Cancer Res. 67, 4303).

Proteasomes are large multi-catalytic protease complexes responsible for degrading the majority of cellular proteins. The 20S-core particle of the 26S proteasome is a barrel-shaped superstructure, and the sites of proteolytic activity reside in the interior.

The eukaryotic proteasome is known to have proteolytic activity that is associated with its β subunits. For example, the β5 subunit is associated with chymotrypsin-like proteolytic activity (cleavage after hydrophobic residues); the β subunit exhibits trypsin-like activity (cleavage after basic residues); and the β1 subunit is responsible for caspase-like activity (cleavage after acidic residues). These three proteolytic properties depend on the presence of an N-terminal threonine (Thr 1) residue. The hydroxyl group of the Thr 1 side chain is responsible for catalyzing cleavage of substrate peptides through nucleophilic attack. Binding pockets adjacent to the N-terminal threonine residue recognize the side chains of substrate peptides to be degraded and confer upon each catalytic site its substrate specificity. The S1 pocket of the β5 subunit is defined by hydrophobic residues, Ala 20, Val 31, Ile 35, Met 45, Ala 49, and Glu 53, and this binding pocket has been shown to be important for substrate specificity and binding of several types of proteasome inhibitors (Smith D M et al. Proteins: Structure, Function, and Bioinformatics (2004) 54, 58; Dou Q P et al. Inflammopharmacology (2008) 16, 208).

Catechol-O-methyl transferase (COMT) is an enzyme widely distributed throughout the body (Mannisto, P. T. and Kaakkola, S., Pharmacol Rev. (1999) 51, 593). Certain endogenous catecholamine neurotransmitters, such as dopamine, noradrenaline and adrenaline, as well as the amino acid L-DOPA and also catecholestrogens are substrates of COMT.

COMT is also able to methylate one or more of the phenolic groups of (−)-EGCG (Zhu, B. T. et al., Drug Metab. Dispos. (2000) 28, 1024; Meng, X. et al. Chem. Res. Toxicol. (2002) 15, 42). In humans, a single gene for COMT encodes both a soluble COMT (S-COMT) and a membrane-bound COMT (MB-COMT).

A single nucleotide polymorphism (G to A) in codon 108 (S-COMT) or 158 (MB-COMT) results in a valine to methionine (Val to Met) substitution, leading to a high- (Val/Val [H/H]), intermediate- (Val/Met [H/L]), or low-activity (Met/Met [L/L]) form of COMT (Lachman, H. M. et al., Pharmacogenetics. (1996) 6, 243.). There is a three-to-four-fold difference in enzyme activity between the high- and low-activity expressed genes (Weinshilboum, R. M. et al., Annu Rev Pharmacol Toxicol. (1999) 39, 19).

The anti-cancer and cancer-preventive effects of green tea and its main constituent EGCG are well documented in the literature including cell culture, animal, epidemiological, and clinical studies.

In addition, a recent case-control study of breast cancer in Asian-American women revealed that women who consumed green tea and who also carried the low activity COMT polymorphism had a reduced risk of breast cancer (Wu, A. H. et al., Cancer Res. (2003) 63, 7526). In contrast, among those who were homozygous for the high activity COMT allele, breast cancer risk did not differ between tea drinkers and non-tea drinkers. These data suggest that EGCG and other tea polyphenols may be less cancer-protective upon methylation.

According to statistics from the American Cancer Society, approximately 180,000 women in the USA are diagnosed with breast cancer each year, of which approximately 30,000 are classified as triple-negative breast cancer (TNBC). TNBC is a specific subtype of breast cancer and TNBC cells do not express the gene signatures for estrogen receptor (ER), progesterone receptor (PR) or Her2/neu. The clinical features of TNBC are a relatively poor prognosis, aggressive behavior, high rate of metastasis and lack of targeted therapies (Miles et al. Breast Cancer Res. (2009) 11, 208; Dent et al. Clin Cancer Res. (2007) 13, 4429). The median survival time is only 15 to 20 months for metastatic TNBC. Molecular characteristics of TNBC include: (i) down-regulated AMP-activated protein kinase (AMPK) signaling pathway; (ii) enriched cancer stem cell population identified with CD44^{high}/CD24^{low} and positivity of aldehyde dehydrogenase 1 (ALDH1); and (iii) over expression of epithelial growth factor receptor (EGFR). TNBC patients experience more than 3 times the risk of recurrence compared with non-triple-negative patients (non-TNBC).

TNBC patients with metastasis have limited treatment options because of the absence of specific targets for chemotherapy. The first-line chemotherapy for TNBC patients includes docetaxel and anthracycline-based chemotherapy (e.g. adriamycin) (Carey et al. Clin Cancer Res. (2007) 13, 2329). Although TNBC patients show sensitivity to these first-line drugs, they are at greater risk for early systemic recurrence and poorer survival compared with their non-TNBC counterparts, since primary and acquired resistance to these drugs occurs in almost 90% of patients with advanced disease (Brown et al. Breast Cancer Res. (2004) 6, R601; Longley and Johnston J. Pathol. (2005) 205, 275). A higher population of cancer stem cells in TNBC may be responsible for the clinical phenomena observed in this aggressive subtype of breast cancer. Therefore treatments which can target cancer stem cells would represent a promising strategy for treatment of TNBC patients.

Metformin, an anti-type II diabetes drug and AMPK activator, shows unique anti-TNBC effects through activation of the AMPK pathway (Liu et al. Cell Cycle (2009) 8, 2031). Metformin inhibits cell proliferation and colony formation and induces apoptosis both in vitro and in vivo selectively in TNBC cell lines (Liu et al. Cell Cycle (2009) 8, 2031; Nalwoga et al. Br. J.l Cancer (2010) 102, 369). At the molecular level, metformin increases phosphorylated/active AMPK (p-AMPK), reduces p-EGFR, and induces apoptosis in TNBC cells (Liu et al. Cell Cycle (2009) 8, 2031). It has also been reported that metformin selectively targets cancer stem cells, and acts together with chemotherapy to inhibit tumor growth of xenografts generated by TNBC cell line (Hirsch et al. Cancer Res. (2009) 69, 7507).

It has been also reported that besides metformin, the natural compounds EGCG and curcumin can induce apoptotic cell death in human colon cancer cells through activation of the AMPK pathway (Lee et al. Ann NY Acad. Sci. (2009) 1171, 489; Park et al. FASEB J. (2010) 24 (Meeting Abstract Supplement), lb260). However, the low absorption, instability and short half-life of EGCG due to metabolic transformations decreases its bioavailability, thus restricting the clinical use of green tea polyphenols. The hydroxyl groups of (−)-EGCG are subject to be modified through biotransformation reactions, including methylation, glucuronidation, and sulfation, resulting in reduced biological activities in vivo. There is a need therefore for tea polyphenols with modified chemical structures which have improved bioavailability in vivo.

There is a need to provide analogs of (−)-EGCG that are able to overcome at least one, but preferably more, of the problems as set forth in the prior art. It would be desirable to provide compounds that are able to overcome the limitations of (−)-EGCG in cancer therapy, as well as to provide polyphenols that inhibit the proteasome, activate AMPK, inhibit cancer cell proliferation, induce apoptosis and/or that are not as prone to deactivation, e.g., through methylation by COMT, compared to (−)-EGCG and other polyphenols of the prior art.

### SUMMARY OF THE INVENTION

Novel compounds and compositions and methods of use thereof for treating cancer, inhibiting proteasomal activity and/or activating AMPK in a cell are provided.

In accordance with an embodiment of the invention, there are provided herein compounds of formula I:

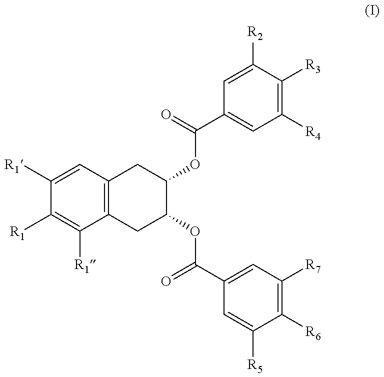

wherein:
R₁, R₁′ and R₁″ are each independently selected from the group consisting of H, alkyl, alkenyl, cycloalkyl, heterocycloalkyl, cycloalkenyl, heterocycloalkenyl, aryl, halogen, OH, an acyloxy group, and NR₈, R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, alkyl, OH, acyloxy, NR₈R₉ or a halogen, wherein
R₈ and R₉ are as defined above;
and analogs thereof; and pharmaceutically acceptable salts thereof.

In an embodiment of compounds of formula I, when R₁, R₁′ and R₁″ are all H and R₂, R₄, R₅ and R₇ are all OH, then R₃ and R₆ are not H or OH; and when R₁, R₁′ and R₁″ are all H and R₂, R₄, R₅ and R₇ are all acyloxy, then R₃ and R₆ are not H or acyloxy.

In accordance with another embodiment of the present invention there are provided compounds of formula Ia:

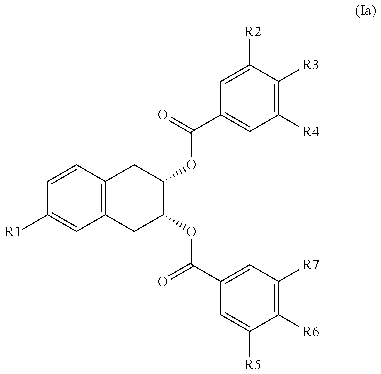

wherein:
R₁ is selected from the group consisting of H, alkyl, alkenyl, cycloalkyl, heterocycloalkyl, cycloalkenyl, heterocycloalkenyl, aryl, halogen, OH, an acyloxy group, and NR₈, R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, alkyl, OH, acyloxy, NR₈R₉ or a halogen, wherein
R₈ and R₉ are as defined above;
and analogs thereof; and pharmaceutically acceptable salts thereof.

In an embodiment of compounds of formula Ia, when R₁, is H and R₂, R₄, R₅ and R₇ are all OH, then R₃ and R₆ are not H or OH; and when R₁ is H and R₂, R₄, R₅ and R₇ are all acyloxy, then R₃ and R₆ are not H or acyloxy.

In another embodiment of the invention there are provided compounds of formula I or Ia, wherein R₁ is selected from the group consisting of H, halogen, OH, and an acyloxy group; R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and R₃ and R₆ are each independently H, alkyl, OH, acyloxy, NR₈R₉ or a halogen; and analogs thereof; and pharmaceutically acceptable salts thereof.

In another embodiment of compounds of formula I or Ia, when R₁, is H and R₂, R₄, R₅ and R₇ are all OH, then R₃ and R₆ are not H or OH; and when R₁ is H and R₂, R₄, R₅ and R₇ are all acyloxy, then R₃ and R₆ are not H or acyloxy.

In accordance with another embodiment of the present invention, there is provided a compound having the structure of formula II:

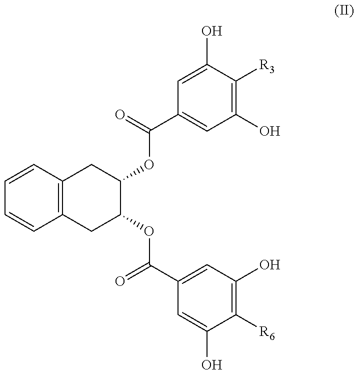

wherein R₃ and R₆ are both H, Br, F, Cl or CH₃; or an analog thereof; and pharmaceutically acceptable salts thereof.

In an embodiment of compounds of formula I₁, R₃ and R₆ are not H.

In accordance with yet another embodiment of the present invention, there is provided a compound having the structure of formula III:

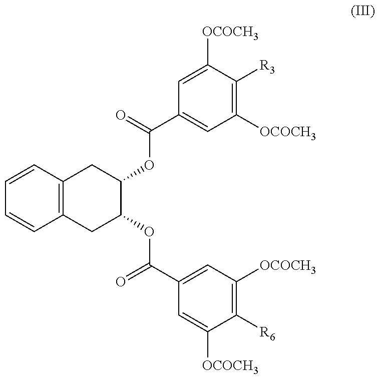

wherein R₃ and R₆ are both OCOCH₃, H, Br, F, Cl or CH₃; or an analog thereof; and pharmaceutically acceptable salts thereof.

In an embodiment of compounds of formula III, R₃ and R₆ are not OCOCH₃ or H.

In accordance with yet another embodiment of the present invention, there is provided a compound having the structure of formula IV:

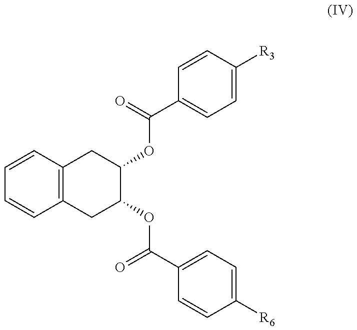

wherein R₃ and R₆ are both OH, OCOCH₃, NHCOOC(CH₃)₃, NH₂ or NHCOCH₃;
or an analog thereof; and pharmaceutically acceptable salts thereof.

In another embodiment of the invention, there is provided a compound having the structure of formula V:

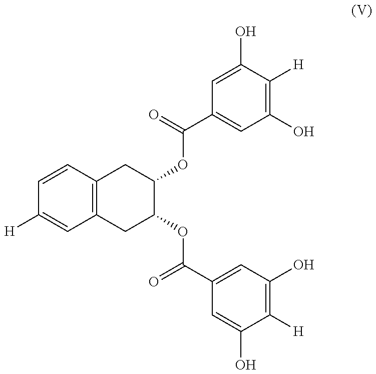 [SMILES: "(V)" ; O=C(O[C@H]1Cc2ccccc2C[C@H]1OC(=O)c1cc(O)cc(O)c1)c1cc(O)cc(O)c1]

or an analog thereof; and pharmaceutically acceptable salts thereof.

In another embodiment of the invention, there is provided a compound having the structure of formula VI:

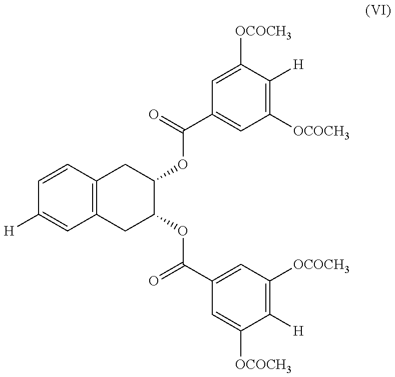

or an analog thereof; and pharmaceutically acceptable salts thereof.

In another embodiment of the invention, there is provided a compound having the structure of formula VII:

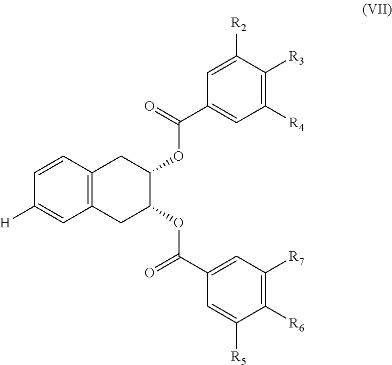

wherein:

### R₂, R₃, R₄, R₅, R₆ and R₇ are F; or

### R₂, R₃, R₅ and R₆ are F, and R₄ and R₇ are H; or

### R₂, R₄, R₅ and R₇ are F, and R₃ and R₆ are H;

or an analog thereof; and pharmaceutically acceptable salts thereof.

In another embodiment of the invention, there are provided compounds having the structure of formulae VIII, IX and X:

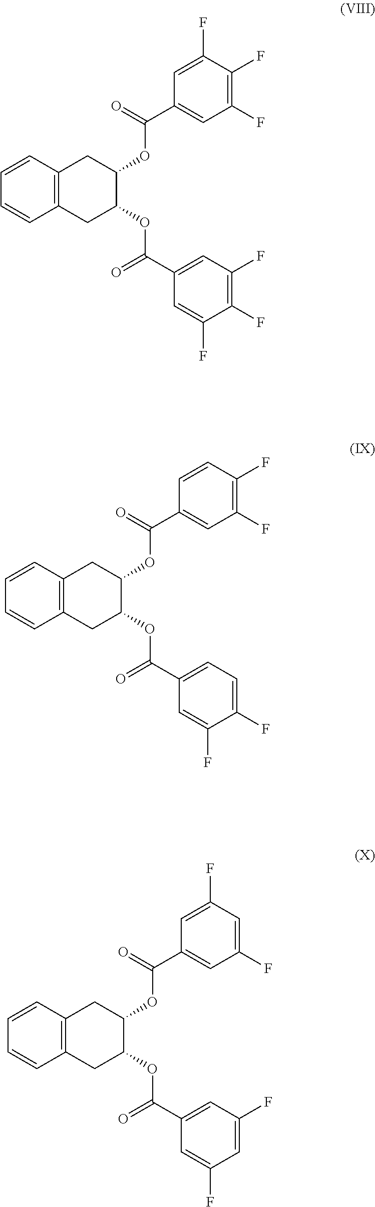 [SMILES: O=C(O[C@H]1Cc2ccccc2C[C@H]1OC(=O)c1cc(F)c(F)c(F)c1)c1cc(F)c(F)c(F)c1 ; O=C(O[C@H]1Cc2ccccc2C[C@H]1OC(=O)c1ccc(F)c(F)c1)c1ccc(F)c(F)c1 ; O=C(O[C@H]1Cc2ccccc2C[C@H]1OC(=O)c1cc(F)cc(F)c1)c1cc(F)cc(F)c1]

or an analog thereof; and pharmaceutically acceptable salts thereof.

In another embodiment of the invention, there are provided compounds having the structure of formula XI:

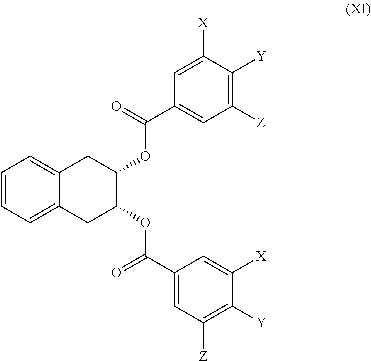

wherein X, Y and Z are each independently H, Br, F, Cl, OH, Me, NH₂, OAc, NHAc or CF₃; or an analog thereof; and pharmaceutically acceptable salts thereof.

In an embodiment of compounds of formula XI, when X and Z are both OH, then Y is not H or OH; and when X and Z are both Oac, then Y is not H or OAc.

In a particular embodiment, in the compounds of formula XI, X and Z are the same.

In an embodiment, there are provided the compounds of formula XI given in Table A.

In another embodiment, there are provided herein compounds having the structures described herein, for example the structures shown in Table 1, Table A, Scheme 1, Scheme 2, and Scheme 3, and analogs and pharmaceutically acceptable salts thereof. In one embodiment, the compound of the invention is an analog of a tea polyphenol.

In an embodiment, there are provided herein compounds having the structures described herein, for example the structures shown in Table 1 and Table A, and analogs and pharmaceutically acceptable salts thereof, wherein the compound is not compound 5, 6, 7, 8, 16, 17, 21 or 22.

In another embodiment, there are provided herein pharmaceutical compositions comprising at least one compound as provided herein and a pharmaceutically acceptable carrier. In an embodiment, the pharmaceutical compositions further comprise at least one additional active ingredient or therapeutic agent. For example, the pharmaceutical compositions may further comprise a second agent which is an anti-cancer therapeutic, a chemotherapeutic agent, an EGFR inhibitor, or a proteasome inhibitor, such as bortezomib (Velcade™), carfilzomib, docetaxel, paclitaxel, cabazitaxel, or an analog thereof. Other non-limiting examples of second agents include Taxol™, vinblastine, vincristine, camptothecin toptecan, etoposid, teniposide, salinosporamide, epigallocatechin gallate and/or erlotinib. In another embodiment, the pharmaceutical compositions may comprise Metformin.

Also provided herein are methods for inhibiting proteasomal activity and/or activating AMPK in a cell, comprising contacting the cell with an effective amount of at least one compound or pharmaceutical composition of the invention, such that proteasomal activity in the cell is inhibited and/or AMPK is activated. The contacting may occur in vitro or in vivo. Compounds and compositions may be administered by a variety of routes, such as orally, parenterally, subcutaneously, intravenously, intramuscularly, intraperitoneally, intraarterially, transdermally, and via mucosal administration. In an aspect, the proteasome may be a 20S proteasome or a 26S proteasome. In a further aspect, the chymotrypsin activity and/or the chymotrypsin-like activity of the 20S proteasome is inhibited.

There is further provided herein a method for treating cancer in a subject, e.g. a human, comprising administering a therapeutically effective amount of at least one compound or composition of the invention to the subject. In an aspect, cancer cell growth is inhibited in the subject, cancer cell apoptosis is induced in the subject, AMPK is activated in the subject, and/or proteasomal activity is inhibited in the subject. In another aspect, the cancer stem cell population, activity of epidermal growth factor receptor (EGFR), or NF-kB, PI3K, Akt and/or mTOR signaling pathways are decreased or inhibited in the subject. In yet another aspect, the CD44^{high}/CD24^{low} cell population is reduced. In another aspect, the compounds and compositions of the invention reduce a CD44^{high}/CD24^{low} cell population, e.g., in TNBC cells.

The cancer may be, for example, prostate cancer, leukemia, lymphoma, hormone-dependent cancer, breast cancer, colon cancer, lung cancer, epidermal cancer, liver cancer, esophageal cancer, stomach cancer, cancer of the brain, multiple myeloma and/or kidney cancer. In a particular embodiment, the cancer is breast cancer, e.g., TNBC. In another embodiment, the cancer is multiple myeloma.

In another embodiment there is provided herein a method for treating a metabolic disorder in a subject, e.g., a human, comprising administering a therapeutically effective amount of at least one compound or composition of the invention to the subject. The metabolic disorder may be, for example, metabolic syndrome, pre-diabetes, insulin resistance, obesity, dyslipidemia or type II diabetes. In an embodiment, the metabolic disorder is treated in the subject via AMPK activation. In an embodiment, glucose homeostasis is improved, glucose metabolism is modulated, and/or lipid metabolism is modulated in the subject. There is also provided a method for modulating glucose metabolism and/or lipid metabolism, comprising administering a therapeutically effective amount of at least one compound or composition to a subject in need thereof, e.g., a subject having pre-diabetes, insulin resistance, obesity, dyslipidemia or type II diabetes.

In another embodiment there is provided herein a method for increasing the response of a disease to a proteasome inhibitor, comprising administering both a therapeutically effective amount of at least one compound or composition of the invention and the proteasome inhibitor to a subject in need thereof. Non-limiting examples of proteasome inhibitors include bortezomib and carfilzomib. In an embodiment, the compound or composition of the invention and the proteasome inhibitor are co-administered. In another embodiment, the compound or composition of the invention and the proteasome inhibitor are administered sequentially.

Methods for synthesizing the compounds described herein are also provided. In an aspect, dihydronaphthalene is reacted with osmium tetroxide, followed by acylation with two or more equivalents of a substituted protected aryl benzoic acid and a dehydrating agent, removal of benzyloxy protecting group in the presence of a catalyst, and optionally reacting the compound with an acylating agent.

### BRIEF DESCRIPTION OF THE DRAWINGS

Particular embodiments of the present invention will now be explained by way of example and with reference to the accompanying drawings, in which:

FIG. 1 shows the structures of (−)-EGCG, (+)-EGCG, (−)-EGC and Pro-EGCG. The nomenclature of the rings is used throughout this specification.

FIG. 2 shows the proteasome inhibition by (−)-EGCG and its analogs. Purified 20S proteasome was incubated with compound 5, 7, 16, or 21 (A) or MDA-MB-231 cell extract (5.7 μg) was incubated with compound 6, 8, 17, or 22 (B) at indicated concentrations for 2 hours, followed by measuring the proteasomal chymotrypsin-like activity. EGCG was used as a comparative standard.

FIG. 3 shows the inhibition of cellular proteasome by EGCG and EGCG analogs. (A) MDA-MB-231 cell extracts (5.7 μg) were incubated with different concentrations of compound 5 or 7 or EGCG for 2 hours, followed by performance of proteasomal chymotrypsin-like activity assay. EGCG was used as a control. (B) MDA-MB-231 cell extracts (5.7 μg) were pre-treated with 10 μM DNC for 20 minutes, followed by co-incubation with compound 5, or 7 or EGCG for 2 h. The proteasomal chymotrypsin-like activity was measured.

FIG. 4 shows the inhibition of cell proliferation by EGCG analogs. Human breast cancer MDA-MB-231 cells were treated with 25 or 50 μM EGCG analogs for 24 hours, followed by MTT assay. Pro-EGCG was used as a comparison.

FIG. 5 shows the effects of DNC on EGCG analogs efficacy against cell proliferation. Human breast cancer MDA-MB-231 cells with high COMT activity were treated with 50 μM EGCG analogs for 24 hours in the absence or presence of 10 μM DNC, followed by MTT assay. Pro-EGCG was used as a comparison.

FIG. 6 shows the accumulation of proteasome substrates upon contacting MDA-MB-231 cells with analogs 6, 8 and Pro-EGCG. MDA-MB-231 cells were treated with 50 μM EGCG analogs for 22 hours. Extracted proteins were subject to Western blotting analysis using antibodies against ubiquitinated proteins and actin.

FIG. 7 shows the effect of compounds 5, 7 and 23 on human multiple myeloma ARP cells (A) or OPM1 cells (B). The cells were treated with Velcade™ alone or with 20 μM of compounds 5, 7 and 23 or in combination with varying doses of Velcade™ for 48 hrs, followed by a MTT assay.

FIG. 8 shows color changes of the MTT assay in a 96 well-plate in the same experiment as FIG. 7A. Deep purple color indicates fully viable cells; light purple color indicates a reduced number of viable cells; and yellowish color indicates an absence of viable cells.

FIG. 9 shows that EGCG analogs 23 and 30 could activate the AMPK signaling pathway, sensitize MDA-MB-231 cells to docetaxel and erlotinib (an EGFR inhibitor), and induce apoptotic cell death. In FIG. 9A, human breast cancer MDA-MB-231 cells were treated with 20 μM of EGCG, Pro-EGCG and EGCG analogs (compounds 5, 7, 23, 30 and 31), or 10 mM of metformin for 3 hrs. Cell lysates were analyzed by Western blot using antibodies of anti-AMPK, p-AMPK, PARP, p-EGFR, EGFR or β-actin. In FIG. 9B, human breast cancer MDA-MB-231 cells were treated with 20 μM of EGCG analogs 23 and 30, 10 nM of docetaxel alone, or combined treatment with compounds 23 and 30 plus docetaxel for 24 hrs. Cell lysates were analyzed by Western blot using antibodies of anti-AMPK, p-AMPK, PARP, p-EGFR, EGFR or β-actin. In FIG. 9C, cells were incubated in medium (0.1% BSA without FBS) containing 20 μM of 23, 30, 2.5 μM of Erlotinib (Eb) alone or the combinations as indicated for 24 hrs. Cell lyses were analyzed by Western blot using antibodies of anti-AMPK, p-AMPK, PARP, p-EGFR, EGFR or β-actin.

FIG. 10 shows that EGCG analogs 23 and 30 can effectively decrease the CD44⁺/CD24⁻ cell population in TNBC cells. The MDA-MB-231 cells were treated with the indicated concentrations of compounds 23 and 30 for 48 hours, followed by flow cytometry analysis. Columns show mean of three experiments; bars, SD; **, p<0.01; *, p<0.1.

FIG. 11 shows that EGCG analogs 23 and 30 inhibited mammosphere formation. MDA-MB-231 cells were seeded in low attached 6-well plates (1000 cells/well) and treated with indicated concentrations of 23 and 30 for 7 days, followed by calculating numbers of mammosphere (A) and taking photos of mammosphere morphology (B). Metformin and EGCG were served as control. *, P<0.1; **, P<0.01. Columns, mean of three experiments; bars, SD.

FIG. 12 shows that EGCG analogs 23 and 30 inhibited breast cancer cell proliferation through activation of AMPK and upregulation of p21. (A) EGCG analogs 23 and 30 inhibited breast cancer cell proliferation. MDA-MB-231 cells were treated with indicated concentrations of 23, 30 or metformin for 24 h, followed by a MTT assay. *, P<0.1; **, P<0.01. Columns, mean of three experiments; bars, SD. (B) EGCG analogs 23 and 30 activated AMPK at a dose-dependent manner, measured by elevated level of phosphor-AMPKα and its downstream proteins of phosphor-Raptor. Treatment with 23 and 30 also showed increased level of p21. MDA-MB-231 cells were treated with indicated concentrations of 23 or 30 for 3 h, followed by Western blot analysis with the indicated antibodies. The numbers underneath the Western results of p-AMPKα indicated normalized phosphor-AMPKα/β-actin ratios. The numbers underneath the Western results of p-AMPKα indicate normalized phosphor-AMPKα/β-actin ratios.

### DETAILED DISCLOSURE OF THE INVENTION

The present invention is directed to polyphenolic compounds useful for inhibiting proteasomal activity and/or activating AMPK, methods of synthesis thereof, pharmaceutical compositions thereof, and use thereof for proteasome inhibition, for activating AMPK, for treating cancer and/or for treating a metabolic disorder. In particular, the polyphenol compounds of the present invention activate AMPK and/or inhibit the chymotrypsin-like activity of a proteasome. The polyphenol compounds of the present invention may be synthesized using methods disclosed herein.

We have modified the chemical structures of tea polyphenols to provide compounds and compositions for cancer therapy. We have previously reported that EGCG potently inhibits the chymotrypsin-like activity of the proteasome in vitro and induces tumor cell growth arrest in the GI phase of the cell cycle (Nam et al. J. Biol. Chem. (2001) 276, 13322). Furthermore, we have established that an ester bond within EGCG may be important for its proteasomal inhibitory properties (Nam et al. J. Biol. Chem. (2001) 276, 13322). Previously, we also synthesized a peracetate-protected EGCG (Pro-EGCG) with improved bioavailability in vivo (Landis-Piwowar et al. Cancer Res. (2007) 67, 4303; Smith et al. Mol. Med. (Cambridge, Mass.) (2002) 8, 382).

We provide herein a series of synthetic EGCG analogs for treating cancer. We have found that EGCG analogs can inhibit cancer cell proliferation, induce apoptosis, inhibit the chymotrypsin-like activity of a proteasome, and/or activate AMPK, in some cases with greater potency than the natural compound EGCG. EGCG analogs also demonstrate effects on breast cancer and multiple myeloma cell lines, e.g., TNBC cell lines.

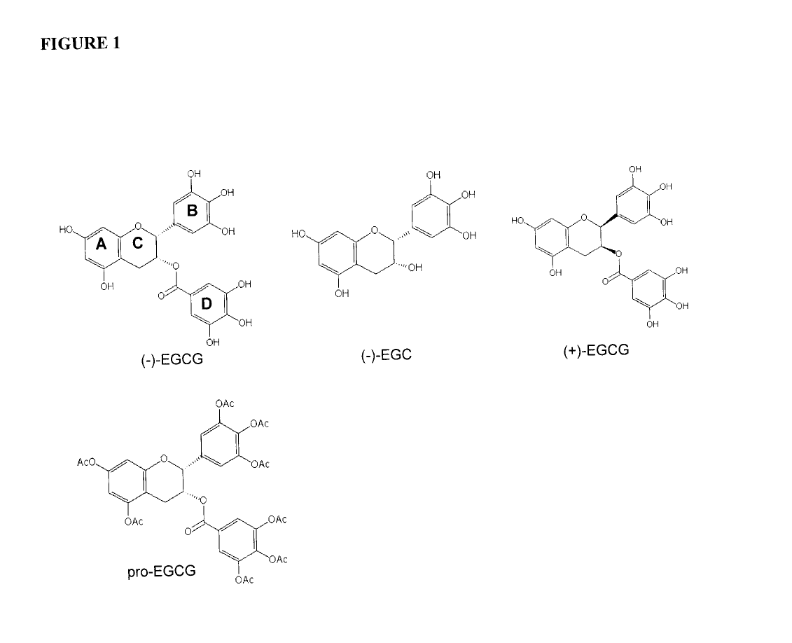

The nomenclature of FIG. 1, whereby the rings of (−)-EGCG are named A, B, C or D, is utilized throughout the specification.

One embodiment of the subject invention is directed to polyphenolic compounds having a similar ring structure to green tea polyphenols. More particularly, in an embodiment, the compounds of the present invention possess an adequate number of phenol substituents or carbonyl oxygens to ensure activation of AMPK, and/or favorable binding and inhibition of the proteasome. In an embodiment, analogs of green tea polyphenols are provided.

In accordance with another embodiment of the invention, the polyphenol analogs disclosed herein are symmetrical and do not contain the phenolic substitution pattern of epigallocatechin or epigallocatechin gallate.

In accordance with another embodiment of the present invention, there is provided a compound having the structure of formula I:

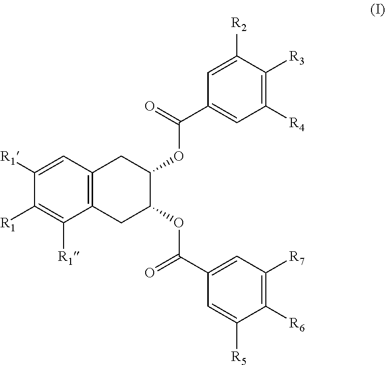

wherein:
R₁, R₁′ and R₁″ are each independently selected from the group consisting of H, alkyl, alkenyl, cycloalkyl, heterocycloalkyl, cycloalkenyl, heterocycloalkenyl, aryl, halogen, OH, an acyloxy group, and NR₈, R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, alkyl, OH, acyloxy, NR₈R₉ or a halogen, wherein
R₈ and R₉ are as defined above;
or an analog thereof; and pharmaceutically acceptable salts thereof.

In an embodiment of compounds of formula I, when R₁, R₁′ and R₁″ are all H and R₂, R₄, R₅ and R₇ are all OH, then R₃ and R₆ are not H or OH; and when R₁, R₁′ and R₁″ are all H and R₂, R₄, R₅ and R₇ are all acyloxy, then R₃ and R₆ are not H or acyloxy.

In another embodiment of the invention, there are provided compounds having the structure of formula Ia:

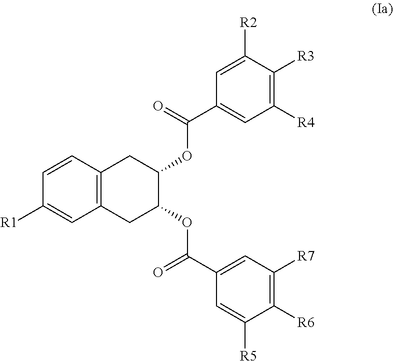

wherein:
R₁ is selected from the group consisting of H, alkyl, alkenyl, cycloalkyl, heterocycloalkyl, cycloalkenyl, heterocycloalkenyl, aryl, halogen, OH, an acyloxy group, and NR₈, R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, alkyl, OH, acyloxy, NR₈R₉ or a halogen, wherein
R₈ and R₉ are as defined above;
or an analog thereof; and pharmaceutically acceptable salts thereof.

In an embodiment of compounds of formula Ia, when R₁, is H and R₂, R₄, R₅ and R₇ are all OH, then R₃ and R₆ are not H or OH; and when R₁ is H and R₂, R₄, R₅ and R₇ are all acyloxy, then R₃ and R₆ are not H or acyloxy.

In another embodiment of the invention there are provided compounds of formula (I) or (Ia), wherein R₁ is selected from the group consisting of H, halogen, OH, and an acyloxy group; R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and R₃ and R₆ are each independently H, alkyl, OH, acyloxy, NR₈R₉ or a halogen; and pharmaceutically acceptable salts thereof.

In yet another embodiment of compounds of formula (I) or (Ia), when R₁, is H and R₂, R₄, R₅ and R₇ are all OH, then R₃ and R₆ are not H or OH; and when R₁ is H and R₂, R₄, R₅ and R₇ are all acyloxy, then R₃ and R₆ are not H or acyloxy.

In accordance with another embodiment of the present invention, there is provided a compound having the structure of formula II:

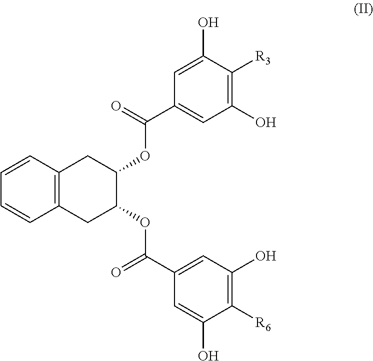

wherein R₃ and R₆ are both H, Br, F, Cl or CH₃; or an analog thereof; and pharmaceutically acceptable salts thereof.

In an embodiment of compounds of formula I₁, R₃ and R₆ are not H.

In accordance with yet another embodiment of the present invention, there is provided a compound having the structure of formula III:

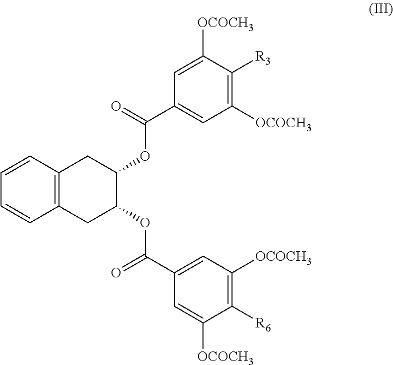

wherein R₃ and R₆ are both OCOCH₃, H, Br, F, Cl or CH₃; or an analog thereof; and pharmaceutically acceptable salts thereof.

In an embodiment of compounds of formula III, R₃ and R₆ are not OCOCH₃ or H.

In accordance with yet another embodiment of the present invention, there is provided a compound having the structure of formula IV:

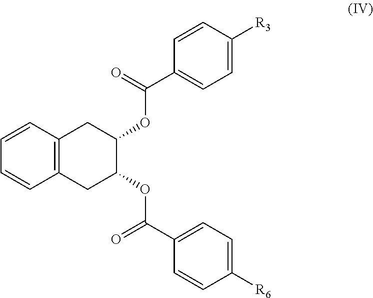

wherein R₃ and R₆ are both OH, OCOCH₃, NHCOOC(CH₃)₃, NH₂ or NHCOCH₃; or an analog thereof; and pharmaceutically acceptable salts thereof.

In another embodiment of the invention, there is provided a compound having the structure of formula V:

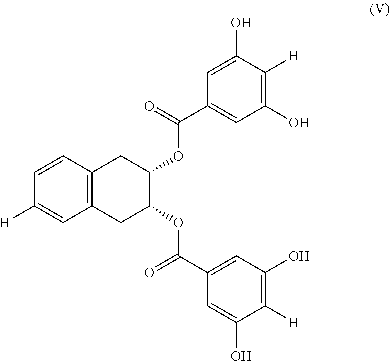 [SMILES: O=C(O[C@H]1Cc2ccccc2C[C@H]1OC(=O)c1cc(O)cc(O)c1)c1cc(O)cc(O)c1]

or an analog thereof; and pharmaceutically acceptable salts thereof.

In another embodiment of the invention, there is provided a compound having the structure of formula VI:

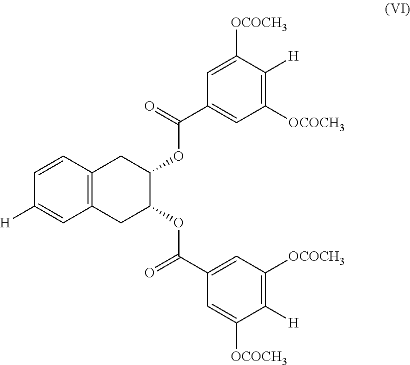

or an analog thereof; and pharmaceutically acceptable salts thereof.

In another embodiment of the invention, there is provided a compound having the structure of formula VII:

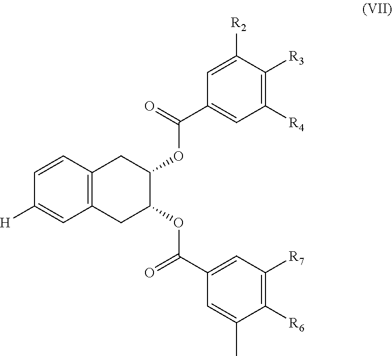

wherein:

### R₂, R₃, R₄, R₅, R₆ and R₇ are F; or

### R₂, R₃, R₅ and R₆ are F, and R₄ and R₇ are H; or

### R₂, R₄, R₅ and R₇ are F, and R₃ and R₆ are H; or

an analog thereof; and pharmaceutically acceptable salts thereof.

In another embodiment of the invention, there are provided compounds having the structure of formulae VIII, IX and X:

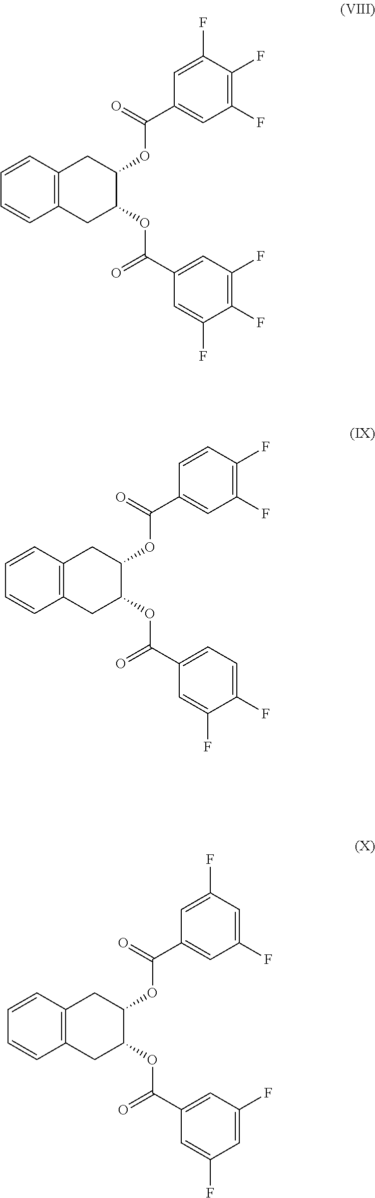 [SMILES: O=C(O[C@H]1Cc2ccccc2C[C@H]1OC(=O)c1cc(F)c(F)c(F)c1)c1cc(F)c(F)c(F)c1 ; O=C(O[C@H]1Cc2ccccc2C[C@H]1OC(=O)c1ccc(F)c(F)c1)c1ccc(F)c(F)c1 ; O=C(O[C@H]1Cc2ccccc2C[C@H]1OC(=O)c1cc(F)cc(F)c1)c1cc(F)cc(F)c1]

or an analog thereof; and pharmaceutically acceptable salts thereof.

In another embodiment of the invention, there are provided compounds having the structure of formula XI:

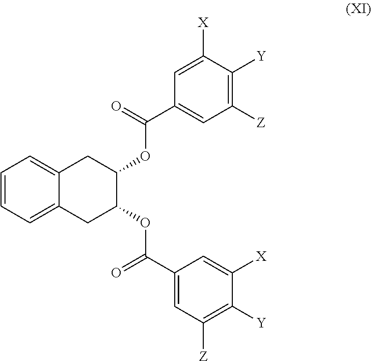

wherein X, Y and Z are each independently H, Br, F, Cl, OH, Me, NH₂, OAc, NHAc or CF₃; or an analog thereof; and pharmaceutically acceptable salts thereof.

In an embodiment of compounds of formula XI, when X and Z are both OH, then Y is not H or OH; and when X and Z are both Oac, then Y is not H or OAc.

In an embodiment, there are provided compounds having the structure of formula XI, as defined above, wherein X and Z are the same.

In another embodiment, there are provided herein compounds having the structures described herein, for example the structures shown in Table 1, Table A, Scheme 1, Scheme 2, and Scheme 3, and analogs and pharmaceutically acceptable salts thereof. In one embodiment, a compound of the invention is an analog of a tea polyphenol. In an embodiment, a compound of the invention is an EGCG analog.

TABLE 1
Representative compounds of the invention.

| Compound # | Structure |
|---|---|
| 1 ((−)-EGCG) | 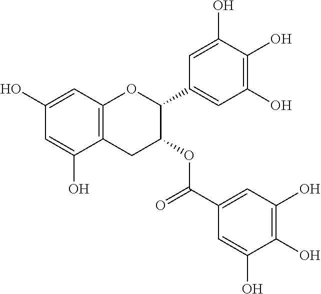 [SMILES: O=C(O[C@@H]1Cc2c(O)cc(O)cc2O[C@@H]1c1cc(O)c(O)c(O)c1)c1cc(O)c(O)c(O)c1] |
| 2 ((−)-EGC) | 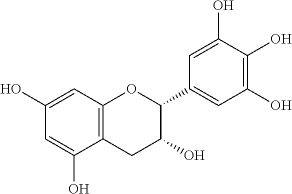 [SMILES: Oc1cc(O)c2c(c1)O[C@H](c1cc(O)c(O)c(O)c1)[C@H](O)C2] |
| 3 ((+)-EGCG) | 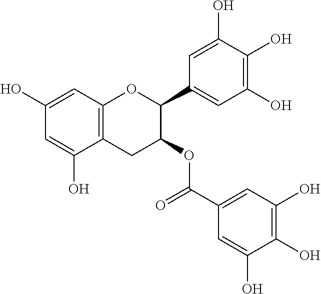 [SMILES: O=C(O[C@H]1Cc2c(O)cc(O)cc2O[C@H]1c1cc(O)c(O)c(O)c1)c1cc(O)c(O)c(O)c1] |
| 4 (Pro-EGCG) | 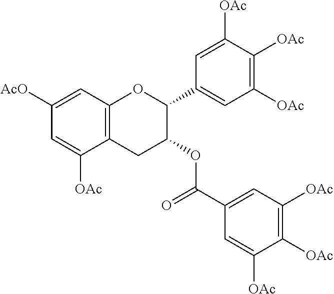 [SMILES: CC(=O)Oc1cc(OC(C)=O)c2c(c1)O[C@H](c1cc(OC(C)=O)c(OC(C)=O)c(OC(C)=O)c1)[C@H](OC(=O)c1cc(OC(C)=O)c(OC(C)=O)c(OC(C)=O)c1)C2] |
| 5: R₃ = OH, R₆ = OH 7: R₃ = H, R₆ = H 23: R₃ = Br, R₆ = Br 25: R₃ = CH₃, R₆ = CH₃ | 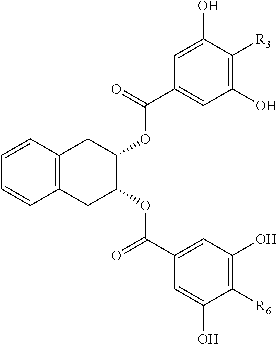 |
| 6: R₃ = OAc, R₆ = OAc 8: R₃ = H, R₆ = H 24: R₃ = Br, R₆ = Br 26: R₃ = CH₃, R₆ = CH₃ | 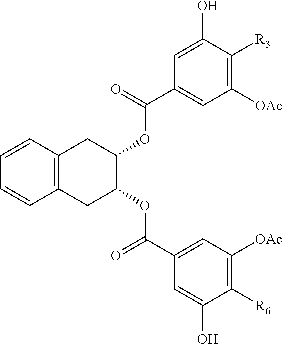 |
| 27: R₃ = OH, R₆ = OH 28: R₃ = OAc, R₆ = OAc 29: R₃ = NHBoc, R₆ = NHBoc 30: R₃ = NH₂, R₆ = NH₂ 31: R₃ = NHAc, R₆ = NHAc | 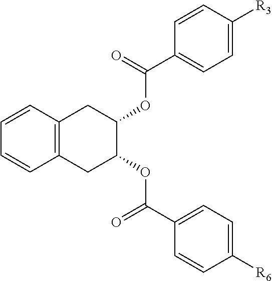 |
| 32 | 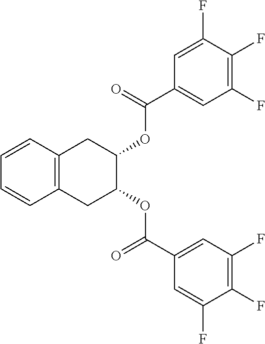 [SMILES: O=C(O[C@H]1Cc2ccccc2C[C@H]1OC(=O)c1cc(F)c(F)c(F)c1)c1cc(F)c(F)c(F)c1] |
| 33 | 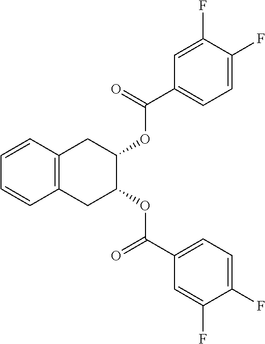 [SMILES: O=C(O[C@H]1Cc2ccccc2C[C@H]1OC(=O)c1ccc(F)c(F)c1)c1ccc(F)c(F)c1] |
| 34 | 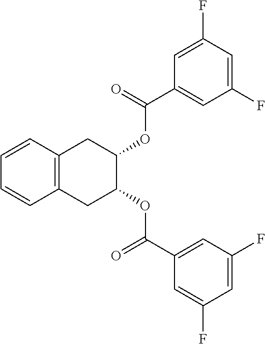 [SMILES: O=C(O[C@H]1Cc2ccccc2C[C@H]1OC(=O)c1cc(F)cc(F)c1)c1cc(F)cc(F)c1] |
| 16 | 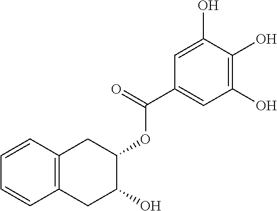 [SMILES: O=C(O[C@H]1Cc2ccccc2C[C@H]1O)c1cc(O)c(O)c(O)c1] |
| 17 | 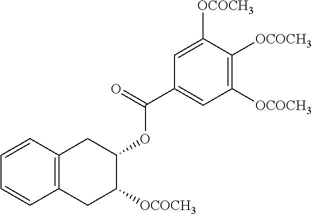 |
| 21 | 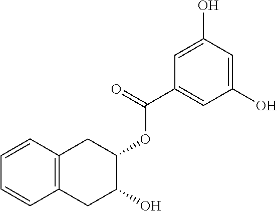 [SMILES: O=C(O[C@H]1Cc2ccccc2C[C@H]1O)c1cc(O)cc(O)c1] |
| 22 | 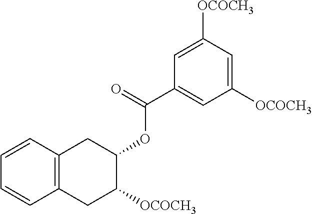 |

TABLE A
Representative compounds of the invention.

| Compound # | Structure |
|---|---|
| 35: X = Br, Y = Z = H 36: Y = Br, X = Z = H 37: X = Br, Y = H, Z = OH 38: X = Br, Y = H, Z = OAc 39 (= 27^{a}): Y = OH, X = Z = H 40 (= 28^{a}): Y = OAc, X = Y = H 41 (= 25^{a}): Y = Me, X = Z = OH 42 (= 26^{a}): Y = Me, X = Z = OAc 43 (= 32^{a}): X = Y = Z = F 44 (= 33^{a}): X = Y = F, Z = H 45 (= 34^{a}): X = Z = F, Y = H 46: X = Z = H, Y = F 47: X = F, Y = CF₃, Z = H 48: X = CF₃, Y = F, Z = H 49: X = Z = Cl, Y = OH 50: X = Z = Cl, Y = OAc 51: Y = C1, X = Z = H 52: X = C1, Y = Z = H 55 (= 23^{a}): X = Z = OH, Y = Br 56 (= 31^{a}): X = Z = H, Y = NHAc 57 (= 30^{a}): X = Z = H, Y = NH₂ 58 (= 24^{a}): X = Z = OAc, Y = Br | 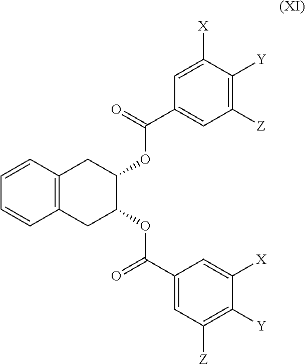 |

^{a}Compound number in Table 1.

In an embodiment, there is provided herein a pharmaceutical composition comprising a compound of the invention (e.g. a compound of Formula I, Ia, II, III, IV, V, VI, VII, VIII, IX, X, or XI; a compound shown in Table 1; a compound shown in Table A; a compound shown in Scheme 1; a compound shown in Scheme 2; a compound shown in Scheme 3; or an EGCG analog) and one or more than one pharmaceutically acceptable carriers. Many pharmaceutically acceptable carriers are known in the art. It will be understood by those in the art that a pharmaceutically acceptable carrier must be compatible with the other ingredients of the formulation and tolerated by a subject in need thereof.

In another embodiment, a pharmaceutical composition comprises at least one additional active ingredient including, but not limited to, antioxidants, free-radical scavenging agents, peptides, growth factors, antibiotics, bacteriostatic agents, immunosuppressives, anticoagulants, buffering agents, anti-inflammatory agents, anti-pyretics, time-release binders, anesthetics, steroids and corticosteroids. In yet another embodiment, the pharmaceutical composition comprises at least one additional active ingredient including, but not limited to, other active ingredients commonly used in therapy for cancer such as bortezomib, docetaxel, paclitaxel, cabazitaxel, erlotinib and other natural, modified or synthetic chemotherapeutic agents known in the art. Other non-limiting examples of additional active ingredients which may be included in pharmaceutical compositions of the invention include vinblastine, vincristine, camptothecin, toptecan, etoposid, and teniposide. In a particular embodiment, the pharmaceutical composition of the invention comprises a compound of the invention and bortezomib (e.g. Velcade™) and a pharmaceutically acceptable carrier. In another embodiment, the pharmaceutical composition of the invention comprises bortezomib (Velcade™), Taxol™, Metformin, docetaxel and/or erlotinib and a pharmaceutically acceptable carrier. In another embodiment, the pharmaceutical composition of the invention comprises bortezomib (Velcade™) or Metformin. In another embodiment, the pharmaceutical composition of the invention comprises a therapeutic for diabetes, such as, for example, a biguanide compound, a sulfonyl urea compound, a meglitinide compound, and/or a thiazolidinedione compound.

In an embodiment, the pharmaceutical compositions of the invention comprise a compound of formula I and a pharmaceutically acceptable carrier, optionally in association with at least one additional active agent. In another embodiment, the pharmaceutical compositions of the invention comprise a compound of formula Ia, II, III, IV, V, VI, VII, VIII, IX, X or XI; or a compound shown in Table 1; or a compound shown in Table A; or a compound shown in Scheme 1, 2, or 3; and a pharmaceutically acceptable carrier, optionally in association with at least one additional active agent. In an embodiment, an at least one additional active agent is a therapeutic agent for cancer or a chemotherapeutic agent. In one embodiment, an at least one additional active agent is bortezomib (Velcade™), or docetaxel, or erlotinib.

In an aspect, compounds and compositions provided herein may be used for treating various types of cancer, and/or for inhibiting cancer cell growth. In an embodiment, compounds and compositions provided herein inhibit chymotrypsin-like activity of 20S proteasome and/or 26S proteasome.

In another aspect, compounds and compositions of the invention comprise a compound selected from the group consisting of the compounds described herein, pharmaceutically acceptable salts, analogs, and mixtures thereof. The compound may be an analog of a tea polyphenol, e.g. an analog of EGCG. Pharmaceutically acceptable salts are known in the art and it should be understood that pharmaceutically acceptable salts of the compounds described herein are encompassed by the present invention.

Compositions and formulations of the invention include those suitable for oral, rectal, nasal, topical (including transdermal, buccal and sublingual), vaginal, parental (including subcutaneous, intramuscular, intravenous and intradermal) and pulmonary administration. Compositions of the present invention suitable for oral administration can be presented for example as discrete units such as capsules, cachets or tablets, each containing a predetermined amount of the active ingredient; or as an oil-in-water liquid emulsion, water-in-oil liquid emulsion or as a supplement within an aqueous solution. The active ingredient can also be presented as bolus, electuary, or paste. Formulations suitable for topical administration in the mouth include lozenges comprising the active ingredient, pastilles comprising the active ingredient in gelatin and glycerin, or sucrose and acacia.

Pharmaceutical compositions for topical administration according to the present invention can be formulated for example as an ointment, cream, suspension, lotion, powder, solution, paste, gel, spray, aerosol or oil. Alternatively, a formulation may comprise a patch or a dressing such as a bandage or adhesive plaster impregnated with active ingredients, and optionally one or more excipients or diluents.

Formulations suitable for topical administration to the eye also include eye drops wherein the active ingredient is dissolved or suspended in a suitable carrier, especially a sterile aqueous solvent for the agent. Formulations for rectal administration may be provided as a suppository with a suitable base comprising, for example, cocoa butter or a salicylate. Formulations suitable for vaginal administration can be presented as pessaries, tampons, creams, gels, pastes, foams or spray formulations containing in addition to the agent, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held to the nose. Suitable formulations wherein the carrier is a liquid for administration by nebulizer, include for example aqueous or oily solutions of the agent.

Formulations suitable for parenteral administration include aqueous and non-aqueous isotonic sterile injection solutions which may contain preservatives, buffers, bacteriostatic agents and solutes which render the formulation isotonic with the blood of the patient; and aqueous and nonaqueous sterile suspensions which can include suspending agents and thickening agents, and liposomes or other microparticulate systems which are designed to target the compound to blood components or one or more organs. Extemporaneous injection solutions and suspensions can be prepared from sterile powders, granules and tablets.

It should be understood that in addition to the ingredients particularly mentioned above, the compositions and formulations of this invention can include other agents conventional in the art regarding the type of formulation in question. For example, formulations suitable for oral administration can include such further agents as sweeteners, thickeners, and flavoring agents. It also is intended that the agents, compositions, and methods of this invention be combined with other suitable compositions and therapies.

Various delivery systems are known and can be used to administer a therapeutic agent of the invention, e.g., encapsulation in liposomes, microparticles, microcapsules and the like. Methods of delivery include, but are not limited to, intraarterial, intramuscular, intravenous, intranasal, and oral routes. In a specific embodiment, the compounds and pharmaceutical compositions of the invention can be administered locally to the area in need of treatment; such local administration can be achieved, for example, by local infusion during surgery, by injection, or by means of a catheter.

Therapeutic amounts can be empirically determined and will vary with the pathology being treated, body mass of the subject being treated, and the efficacy and toxicity of the agent. Similarly, suitable dosage formulations and methods of administering the agents can be readily determined by those of skill in the art. For example, a daily dosage can be divided into one, two or more doses in a suitable form to be administered at one, two or more times throughout a time period.

Compounds and pharmaceutical compositions can be administered by any of a variety of routes, such as orally, intranasally, parenterally or by inhalation, and can take the form, for example, of tablets, lozenges, granules, capsules, pills, ampoule, suppositories or aerosol form. They can also be in the form of suspensions, solutions, and emulsions of the active ingredient in aqueous or nonaqueous diluents, syrups, granulates or powders. In addition to an agent of the present invention, pharmaceutical compositions can also contain other pharmaceutically active compounds.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. For example, in an embodiment each tablet may contain from about 2.5 mg to about 500 mg of the active ingredient and each cachet or capsule may contain from about 2.5 to about 500 mg of the active ingredient.

The magnitude of prophylactic or therapeutic dose of a compound of the invention will, of course, vary with the nature of the severity of the condition to be treated and with the particular compound of the invention and its route of administration. It will also vary according to the age, weight and response of the individual patient. In general, the daily dose range for treating cancer lies within the range of from about 0.001 mg to about 100 mg per kg body weight of a mammal, preferably 0.01 mg to about 10 mg per kg, and most preferably 0.1 to 1 mg per kg, in single or divided doses. On the other hand, it may be necessary to use dosages outside these limits in some cases.

For use where a composition for intravenous administration is employed, in an embodiment a suitable dosage range for treating cancer is from about 0.001 mg to about 25 mg (preferably from 0.01 mg to about 1 mg) of a compound of the invention per kg of body weight per day.

In the case where an oral composition is employed, in an embodiment a suitable dosage range for treating cancer is, e.g. from about 0.01 mg to about 100 mg of a compound of the invention per kg of body weight per day, preferably from about 0.1 mg to about 10 mg per kg.

Ideally, the therapeutic agent of the invention should be administered to achieve peak concentrations of the active compound at sites of the disease. Peak concentrations at disease sites can be achieved, for example, by intravenously injecting the agent, optionally in saline, or orally administering, for example, a tablet, capsule or syrup containing the active ingredient.

Advantageously, the compounds and compositions of the invention can be administered simultaneously or sequentially with other drugs or biologically-active agents. Examples include, but are not limited to, antioxidants, free-radical scavenging agents, peptides, growth factors, antibiotics, bacteriostatic agents, immunosuppressives, anticoagulants, buffering agents, anti-inflammatory agents, anti-pyretics, time-release binders, anesthetics, steroids and corticosteroids, other anti-cancer therapeutics and chemotherapeutic agents such as bortezomib (Velcade™), carfilzomib, docetaxel, paclitaxel, cabazitaxel, and erlotinib. Non-limiting examples of other drugs or biologically-active agents include metformin, Taxol™, vinblastine, vincristine, camptothecin toptecan, etoposid, teniposide, salinosporamide, and epigallocatechin gallate and its analogs.

Accordingly, in the methods and uses of the present invention, compounds of the invention can also be administered in combination with other therapeutic agents. In an embodiment, the present invention provides a method of treating cancer, e.g. multiple myeloma or breast cancer, comprising administering to a subject in need thereof an effective amount of a first agent comprising a compound or composition of the invention, and a second agent. The second agent may be, for example, an anti-cancer therapeutic or chemotherapeutic agent, e.g. bortezomib (Velcade™) or docetaxel, or an EGFR inhibitor, e.g. erlotinib. In another embodiment, the present invention provides a method of treating a metabolic disorder, e.g. type II diabetes, comprising administering to a subject in need thereof an effective amount of a first agent comprising a compound or composition of the invention, and a second agent. The second agent may be, for example, an anti-diabetes therapeutic, e.g. metformin.

Administration in combination with another agent includes co-administration (simultaneous administration of a first and second agent) and sequential administration (administration of a first agent, followed by the second agent, or administration of the second agent, followed by the first agent). The combination of agents used within the methods described herein may have a therapeutic additive or synergistic effect on the condition(s) or disease(s) targeted for treatment. The combination of agents used within the methods described herein also may reduce a detrimental effect associated with at least one of the agents when administered alone or without the other agent(s). For example, the toxicity of side effects of one agent may be attenuated by the other, thus allowing a higher dosage, improving patient compliance, or improving therapeutic outcome. Physicians may achieve the clinical benefits of previously recognized drugs while using lower dosage levels, thus minimizing adverse side effects. In addition, two agents administered simultaneously and acting on different targets may act synergistically to modify or ameliorate disease progression or symptoms.

Another aspect of the present invention is directed to methods of inhibiting proteasomal activity. In particular, without limitation the chymotrypsin activity and/or chymotrypsin-like activity of the 20S proteasome may be inhibited.

Another aspect of the present invention is directed to methods of activating AMPK. In an aspect, the cancer stem cell population, activity of epidermal growth factor receptor (EGFR), or NF-kB, PI3K, Akt and/or mTOR signaling pathways are decreased or inhibited in the subject. In yet another aspect, the CD44^{high}/CD24^{low} cell population is reduced. In another aspect, compounds and compositions of the invention reduce the CD44^{high}/CD24^{low} cell population in TNBC cells.

In an aspect, a method of inhibiting proteasomal activity is provided, comprising contacting a cell with a sufficient amount of a compound or composition of the invention.

In another aspect, the present invention provides a method of inhibiting chymotrypsin-like activity of the 20S proteasome, comprising administering to a subject a therapeutically effective amount of a compound or pharmaceutical composition of the present invention.

In an aspect, a method of activating AMPK is provided, comprising contacting a cell with a sufficient amount of a compound or composition of the invention.

In accordance with another embodiment of the present invention, there is provided a method of treating cancer, comprising administering to a subject a therapeutically effective amount of a compound or pharmaceutical composition of the present invention.

A cancer to be treated in accordance with an embodiment of the present invention may be selected from the group consisting of, but not limited to, prostate cancer, leukemia, lymphoma, hormone-dependent cancers, breast cancer, colon cancer, lung cancer, epidermal cancer, liver cancer, esophageal cancer, stomach cancer, cancer of the brain, and cancer of the kidney. In one embodiment, the cancer is multiple myeloma. In another embodiment, the cancer is breast cancer, e.g., TNBC.

In another embodiment of the present invention, there is provided herein a method of inhibiting tumor cell growth, comprising administering to a subject a therapeutically effective amount of a compound or pharmaceutical composition of the present invention.

In accordance with another embodiment of the present invention, there is provided a method of treating a disease by activating AMPK, comprising administering to a subject a therapeutically effective amount of a compound or pharmaceutical composition of the present invention.

Adenosine 5′-Monophosphate-Activated Protein Kinase (AMP-activated protein kinase or AMPK) activators are believed to play a key role in regulation of carbohydrate and fat metabolism in mammals, including humans. The net effects of AMPK activation may include inhibition of hepatic gluconeogenesis, cholesterol and triglyceride synthesis in liver, and/or enhancement in muscle glucose transport, insulin sensitivity or fatty acid oxidation in muscle and liver. The AMPK system is also a probable target of known antidiabetic compounds such as metformin. It is known that activation of the AMPK signaling system can have beneficial effects. For example, it is expected that in liver, decreased expression of gluconeogenic enzymes would reduce hepatic glucose output and improve overall glucose homeostasis. Both direct inhibition and/or reduced expression of key enzymes in lipid metabolism is expected to lead to decreased fatty acid and cholesterol synthesis and increased fatty acid oxidation. Stimulation of AMPK in skeletal muscle is expected to increase glucose uptake and fatty acid oxidation, resulting in improvement of glucose homeostasis. It is also expected that due to a reduction in intra-myocyte triglyceride accumulation, AMPK activation would lead to improved insulin action.

Accordingly, it is expected that compounds and compositions of the present invention, which are useful as AMPK activators, will be useful to treat conditions associated with AMPK dysregulation, e.g., metabolic disorders. In an embodiment, there is provided a method of activating AMPK in a subject, thereby treating a metabolic disorder, e.g., a condition selected from the group consisting of metabolic syndrome, pre-diabetes, insulin resistance, obesity, dyslipidemia and type II diabetes, the method comprising administering to a subject a therapeutically effective amount of a compound or pharmaceutical composition of the present invention. In an embodiment, glucose uptake into cells is increased, and/or glucose homeostasis is improved.

In another embodiment, there is provided a method of modulating glucose metabolism in a subject, comprising administering to a subject a therapeutically effective amount of a compound or pharmaceutical composition of the present invention. Modulation of glucose metabolism may include, for example, increasing glucose uptake in muscle cells, decreasing glucose neogenesis in hepatic cells, and/or improving glucose homeostasis.

In another embodiment, there is provided a method of modulating lipid metabolism in a subject, comprising administering to a subject a therapeutically effective amount of a compound or pharmaceutical composition of the present invention. Modulation of lipid metabolism may include, for example, decreasing total serum cholesterol, serum LDL-cholesterol, and/or serum triglycerides.

In another embodiment, there is provided a method of treating or preventing a condition selected from the group consisting of metabolic syndrome, pre-diabetes, insulin resistance, obesity, dyslipidemia and type II diabetes, in a subject, comprising administering to a subject a therapeutically effective amount of a compound or pharmaceutical composition of the present invention. Without wishing to be bound by theory, it is contemplated that compounds and compositions of the invention will treat or prevent these conditions via activation of AMPK.

In another embodiment, there is provided a method of treating or preventing a disease characterized by decreased AMPK activity, comprising administering a therapeutically effective amount of a compound or pharmaceutical composition of the present invention to a subject in need thereof, such that AMPK activity is increased.

As used herein, “metabolic disorders” include, but are not limited to, metabolic syndrome, diabetes, type II diabetes, type I diabetes, insulin resistance, hyperinsulinemia, abnormal glucose tolerance, obesity, adiposis hepatica, hyperuricacidemia, arthrolithiasis, hyperlipemia, hypercholesteremia, atherosclerosis or hypertension. The common characteristic of these diseases is a metabolism disorder of glucose, lipid and protein. It is contemplated that any metabolic disorder associated with AMPK dysregulation or treated or prevented by AMPK activation may be treated by the compounds and compositions of the invention. Other related conditions or diseases which may be treated or prevented by compounds and compositions of the invention include, without limitation, hyperglycemia, reduced insulin sensitivity, insulin resistance syndrome, insufficient glucose uptake in muscle cells, insulin oversecretion, hepatic ischemia-reperfusion injury, and ischemia.

For the purpose of the present invention the following terms are defined below:

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “inhibition” is intended to mean a substantial slowing, interference, suppression, prevention, delay and/or arrest of a chemical or biochemical action.

The term “pharmacological inhibition” is intended to mean a substantial slowing, interference, suppression, prevention, delay and/or arrest of a chemical action which is caused by an effective amount of a compound, drug, or agent.

The term “inhibitor” is intended to mean a compound, drug, or agent that substantially slows, interferes, suppresses, prevents, delays and/or arrests a chemical action.

The term “polyphenol” is intended to mean a compound with more than one phenolic moiety. A phenolic compound is an aromatic compound containing an aromatic nucleus to which is directly bonded at least one hydroxyl group. The term polyphenol includes, without limitation, (−)EGCG, (−)EGC, (−)ECG, and (−)EC, such as those that can be extracted from leaves of the tea plant Camellia sinensis, and analogs thereof; as well as structurally similar synthetic analogs.

The term “per-acetate” or “per-acetylated” or “per-acylated”, as used herein is intended to mean a polyphenol that is connected by a group such that all the hydroxyl groups of the polyphenol are acylated.

The term “alkyl group”, as used herein, is understood as referring to a saturated, monovalent unbranched or branched hydrocarbon chain. Examples of alkyl groups include, but are not limited to, C₁₋₁₀ alkyl groups. Examples of C₁₋₁₀ alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, nonyl and decyl.

The term “aryl”, as used herein, is understood as referring to 5-, 6- and 7- or more membered aromatic groups, for example phenyl or naphthyl, that may include from zero to four heteroatoms selected independently from O, N and S in the ring, for example, pyrrolyl, furyl, thiophenyl, imidazolyl, oxazole, thiazolyl, triazolyl, pyrazolyl, pyridyl, pyrazinyl, pyridazinyl and pyrimidinyl, and the like. Those aryl groups having heteroatoms in the ring structure may also be referred to as “aryl heterocycles” or “heteroaryl”. The aromatic ring can be substituted at one or more ring positions. Aryl groups can also be part of a polycyclic group. For example, aryl groups include fused aromatic moieties such as naphthyl, anthracenyl, quinolyl, indolyl, and the like.

The term “acyl group” is intended to mean a group having the formula RC═O, wherein R is an alkyl, alkenyl, cycloalkyl, heterocycloalkyl, cycloalkenyl, heterocycloalkenyl, or an aryl group.

The term “alkenyl” refers to a straight or branched chain alkyl moiety having two or more carbon atoms (e.g., two to six carbon atoms, C₂₋₆ alkenyl) and having in addition one double bond, of either E or Z stereochemistry where applicable. This term would include, for example, vinyl, 1-propenyl, 1- and 2-butenyl, 2-methyl-2-propenyl, etc.

The term “cycloalkyl” refers to a saturated alicyclic moiety having three or more carbon atoms (e.g., from three to six carbon atoms) and which may be optionally benzofused at any available position. This term includes, for example, cyclopropyl, cyclobutyl, cyclopentyl, cyclohexyl, indanyl and tetrahydronaphthyl.

The term “heterocycloalkyl” refers to a saturated heterocyclic moiety having three or more carbon atoms (e.g., from three to six carbon atoms) and one or more heteroatom from the group N, O, S (or oxidised versions thereof) and which may be optionally benzofused at any available position. This term includes, for example, azetidinyl, pyrrolidinyl, tetrahydrofuranyl, piperidinyl, indolinyl and tetrahydroquinolinyl.

The term “cycloalkenyl” refers to an alicyclic moiety having three or more carbon atoms (e.g., from three to six carbon atoms) and having in addition one double bond. This term includes, for example, cyclopentenyl or cyclohexenyl.

The term “heterocycloalkenyl” refers to an alicyclic moiety having from three to six carbon atoms and one or more heteroatoms from the group N, O, S (or oxides thereof) and having in addition one double bond. This term includes, for example, dihydropyranyl.

The term “halogen” means a halogen atom such as fluorine, chlorine, bromine, or iodine.

The term “optionally substituted” means optionally substituted with one or more of the aforementioned groups (e.g., alkyl, aryl, heteroaryl, acyl, alkenyl, cycloalkyl, heterocycloalkyl, cycloalkenyl, heterocycloalkenyl, or halogen), at any available position or positions.

The term “analog” is intended to mean a compound that is similar or comparable, but not identical, to a reference compound, i.e. a compound similar in function, structure, properties and/or appearance to the reference compound. For example, the reference compound can be a reference green tea polyphenol and an analog is a substance possessing a chemical structure or chemical properties similar to those of the reference green tea polyphenol. As used herein, an analog is a chemical compound that may be structurally related to another but differs in composition (for example as in the replacement of one atom by an atom of a different element or in the presence of a particular functional group). An analog may be derived from a natural source or be prepared using chemical synthesis.

The term “cancer” is intended to mean any cellular trait or neoplasia, associated with the loss of normal controls which results in unregulated growth, lack of differentiation and ability to invade or lead to invasion of local tissues and metastases. More specifically, cancer is intended to include, without limitation, prostate cancer, leukemia, lymphoma, hormone-dependent cancers, breast cancer, colon cancer, lung cancer, epidermal cancer, liver cancer, esophageal cancer, stomach cancer, cancer of the brain, cancer of the kidney, multiple myeloma and TNBC, as well as premalignant conditions such as smoldering multiple myeloma or high-grade prostatic intraepithelial neoplasia.

The terms “treatment” or “treating” are intended to mean obtaining a desired pharmacologic and/or physiologic effect, such as inhibition of cancer cell growth or induction of apoptosis of a cancer cell or an improvement in a disease condition in a subject or improvement of a symptom associated with a disease or a medical condition in a subject. The effect may be prophylactic in terms of completely or partially preventing a disease or symptom associated therewith and/or may be therapeutic in terms of a partial or complete cure for a disease and/or the pathophysiologic effect attributable to the disease. “Treatment” as used herein covers any treatment of a disease in a mammal and includes: (a) preventing a disease or condition (such as preventing cancer) from occurring in an individual who may be predisposed to the disease but has not yet been diagnosed as having it; (b) inhibiting the disease, (e.g., arresting its development); or (c) relieving the disease (e.g., reducing symptoms associated with the disease).

The term “biological activity” is intended to mean having the ability to inhibit cell growth, induce apoptosis, activate AMPK, suppress transforming activity in cancer cells, and/or inhibit the proteasome, e.g., inhibit the chymotrypsin-like activity of the proteasome. “Biological activity” also means having therapeutic efficacy and/or the ability to treat cancer or a metabolic disorder in a subject.

The term “therapeutically effective” is intended to mean an amount of a compound sufficient to substantially improve a symptom associated with a disease or a medical condition or to improve, ameliorate or reduce the underlying disease or medical condition. For example, in the treatment of cancer, a compound which decreases, prevents, delays, suppresses, or arrests any symptom of the disease would be therapeutically effective. A therapeutically effective amount of a compound may provide a treatment for a disease such that the onset of the disease is delayed, hindered, or prevented, or the disease symptoms are ameliorated, or the term of the disease is altered.

The term “chymotrypsin-like activity” refers to the ability of the eukaryotic proteasome β subunit to cleave amino acid sequences after hydrophobic residues, and is intended to include chymotrypsin activity.

AMP-activated protein kinase (AMPK) is a physiological cellular energy sensor, which is known to suppress cell proliferation, induce apoptosis and reduce the stem cell population in cancer cells. The term “AMPK activation” is intended to mean activation of the AMPK signaling pathway. It will be understood by those skilled in the art that such activation can be direct or indirect, e.g., activation may be effected through a change in the phosphorylation state of the kinase, through action on a downstream target of the kinase, and so on. For example, non-limiting examples of possible molecular targets of the EGCG analogs of the invention include activation of AMPK signaling, decrease of the cancer stem cell population, reduction in the CD44^{high}/CD24^{low} cell population, down-regulating activity of EGFR, and/or suppression of NF-kB, PI3K, Akt and/or mTOR pathways which are downstream of AMPK signaling.

As used herein, the term “subject” includes mammals, including humans.

When the compounds of this invention are administered in combination with other agents, they may be administered sequentially or concurrently to an individual. Alternatively, pharmaceutical compositions according to the present invention may be comprised of a combination of analogs of the present invention, as described herein, and another therapeutic or prophylactic agent known in the art.

It will be understood that a specific “effective amount” for any particular in vivo or in vitro application will depend upon a variety of factors including the activity of the specific compound employed, the age, body weight, general health, sex, and/or diet of the individual, time of administration, route of administration, rate of excretion, drug combination and the severity of the particular disease being treated. For example, the “effective amount” may be the amount of polyphenol compound of the invention necessary to achieve inhibition of proteosomal chymotrypsin-like activity in vivo or in vitro.

The terms “UPS”, “proteasome” and “proteasomal” are used interchangeably throughout the specification.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include citric acid, lactic acid, tartaric acid, fatty acids, and the like. Pharmaceutically acceptable salts are known in the art.

Salts may also be formed with bases. Such salts include salts derived from inorganic or organic bases, for example alkali metal salts such as magnesium or calcium salts, and organic amine salts such as morpholine, piperidine, dimethylamine or diethylamine salts.

As used herein, the term “pharmaceutically acceptable carrier” includes any and all solvents such as phosphate buffered saline, water, saline, dispersion media, coatings, antibacterial and antifungal agents, isotonic and absorption delaying agents and the like. The use of such media and agents for pharmaceutically active substances is well known in the art. Supplementary active ingredients can also be incorporated into the compositions. The pharmaceutical compositions of the invention can be formulated according to known methods for preparing pharmaceutically useful compositions.

Formulations are described in a number of sources which are well known and readily available to those skilled in the art. For example, Remington's Pharmaceutical Science (Martin E W (1995) Easton Pa., Mack Publishing Company, 19th ed.) describes formulations which can be used in connection with the subject invention.

The present description refers to a number of chemical terms and abbreviations used by those skilled in the art. Nevertheless, definitions of selected terms are provided for clarity and consistency.

### Abbreviations:

OsO4: Osmium tetroxide; NMO: N-Methylmorpholine-N-Oxide; Ac₂O: Acetic anhydride; Py: Pyridine; TFA: Trifluoroacetic acid; DIPEA: N,N-Diisopropylethylamine; DMAP: 4-Dimethylaminopyridine; DCC: 1,3-Dicyclohexylcarbodiimide; Bn: Benzyloxy; MeOH: Methanol; TLC: Thin Layer Chromatography; NMR: Nuclear Magnetic Resonance; MS: Mass Spectroscopy; ESI: Electrospray Ionization; FAB: Fast Atom Bombardment; PEG: Polyethylene Glycol; DMF: Dimethylformamide; DMSO: Dimethyl Sulfoxide; THF: Tetrahydrofuran; DNC: 3,5-dinitrocatechol; (−)-EGCG: (−)-Epigallocatechin gallate; Pro-EGCG: (−)-Epigallocatechin gallate octa-acetate; EtOAC: Ethyl acetate; MOM: methoxy methyl; DCM: dichloromethane; TMS: tetramethylsilane; QTOF: quadrupole time-of-flight; N-boc: N-tert-butoxycarbonyl; Suc-LLVY-AMC: N-Succinyl-Leu-Leu-Val-Tyr-AMC (AMC=7-amino-4-methylcoumarin).

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Methods of Synthesis

Compounds of the invention can be prepared according to the synthetic routes outlined below and by following the methods described herein.

The preparation of the compounds of the present invention is illustrated in Schemes 1, 2, and 3. With reference to compounds 5 and 7 and their respective per-acetates 6 and 8, 1,4-Dihydronaphthalene 11 was dihydroxylated with osmium tetraoxide affording the cis-diol 12. Compound 12 is treated with DCC/DMAP and one molar equivalent of benzyl-protected gallic acid affording the corresponding monobenzoate 14. When more than two equivalents of benzyl-protected gallic acid is used in the reaction sequence, the dibenzoate 15 is obtained. Removal of the O-benzyl protecting group of compounds 14 and 15 by palladium catalyzed hydrogenolysis gave compounds 16 and 5 respectively. Acetylation of compounds 16 and 5 gave the corresponding acetates 17 and 6. A similar reaction sequence of compound 12 with 3,5-dibenzyloxybenzoic acid gave the series 21 and 7 which were converted to their respective acetates 22 and 8. A similar approach was used for the synthesis of compounds in Table A.

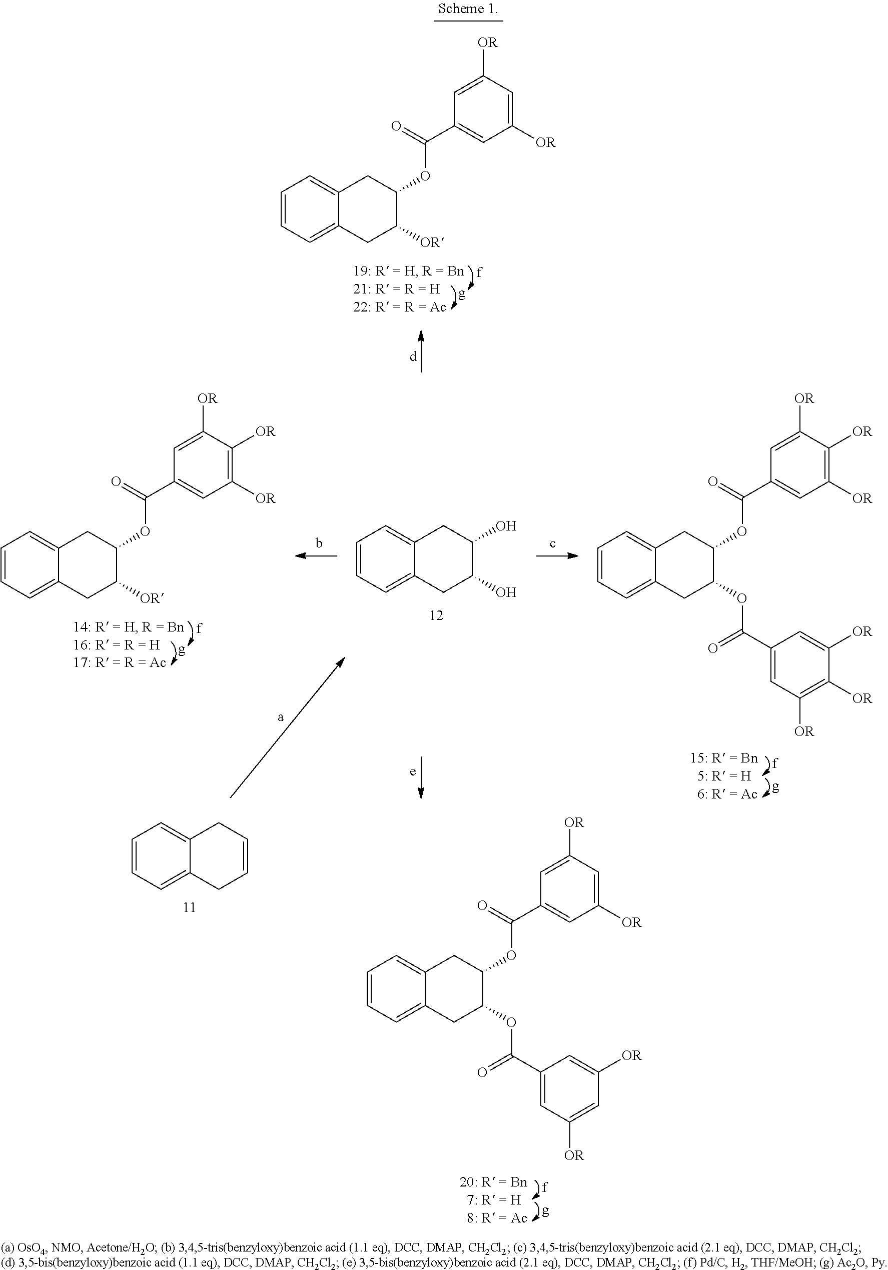

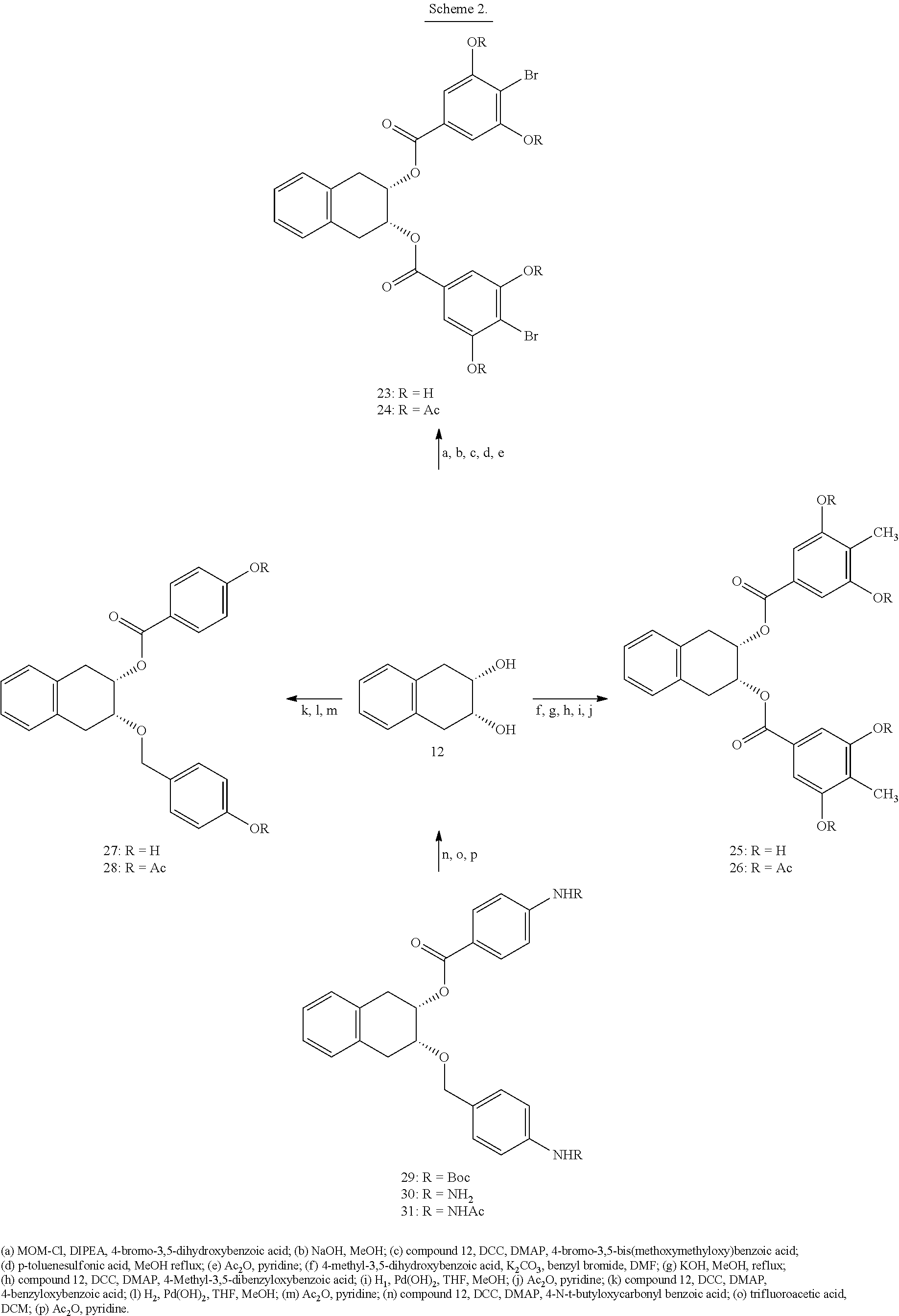

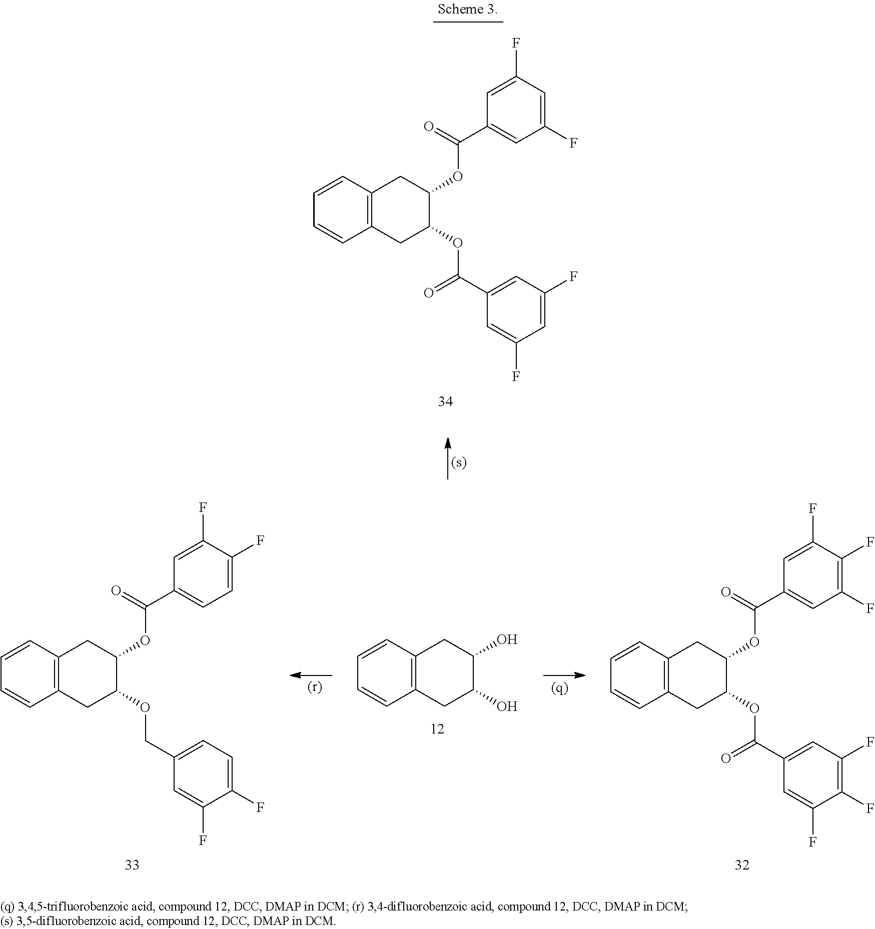 [SMILES: O=C(O[C@H]1Cc2ccccc2C[C@H]1OC(=O)c1cc(F)cc(F)c1)c1cc(F)cc(F)c1 ; O=C(O[C@H]1Cc2ccccc2C[C@H]1OCc1ccc(F)c(F)c1)c1ccc(F)c(F)c1 ; O[C@@H]1Cc2ccccc2C[C@@H]1O ; O=C(O[C@H]1Cc2ccccc2C[C@H]1OC(=O)c1cc(F)c(F)c(F)c1)c1cc(F)c(F)c(F)c1]

### EXPERIMENTAL METHODS

### General Methods.

Starting materials and reagents, purchased from commercial suppliers, were used without further purification. Anhydrous methylene chloride was distilled under nitrogen from CaH₂. Anhydrous DMF was distilled under vacuum from CaH₂. Reaction flasks were flame-dried under a stream of N₂. All moisture-sensitive reactions were conducted under a nitrogen atmosphere. Flash chromatography was carried out using silica-gel 60 (70-230 mesh). The melting points were uncorrected. ¹H-NMR and ¹³C NMR (300 MHz) spectra were measured with TMS as an internal standard when CDCl₃, CD₃OD and acetone-d₆ were used as solvent. High-resolution (ESI) MS spectra were recorded using a QTOF-2 Micromass spectrometer.

### Biological Assays

### Materials.

Purified rabbit 20S proteasome and fluorogenic substrate Suc-LLVY-AMC for the proteasomal chymotrypsin like (CT-like) activity were obtained from Calbiochem Inc. (San Diego, Calif.). Fetal bovine serum (FBS) was from Tissue Culture Biologicals (Tulare, Calif.). Penicillin and streptomycin were purchased from Invitrogen Co. (Carlsbad, Calif.). RPMI 1640 medium was purchased from Invitrogen Co. (Carlsbad, Calif.). MTT (3-4,5-dimethylthiazol-2-yl-2.5-diphenyl-tetrazolium bromide) was purchased from Sigma-Aldrich.

### Cell Culture.

Human breast cancer MDA-MB-231 cells were purchased from American Type Culture Collection (Manassas, Va.) and grown in RPMI 1640 or D-MEM/F-12 medium supplemented with 10% FBS, 100 units/ml of penicillin, and 100 μg/ml of streptomycin as described by Chen et al. Cancer Res. (2006) 66, 10425. Cells were maintained at 37° C. and 5% CO₂.

Human multiple myeloma cells (Arp and Opm1) cells, kindly provided by Dr. Ramesh Batchu (Wayne State University), were grown in RPMI-1640 medium supplemented with 10% FBS, 100 units/ml of penicillin, and 100 μg/ml of streptomycin. Cells were maintained at 37° C. in 5% CO₂ (Mujtaba T, et al., Int J Mol Med. (2012) 29(1):102-6).

### MTT Assay.

Cells were grown in a 96-well plate. Triplicate wells of cells were treated with indicated concentrations of EGCG or EGCG analogs for 24 h. After aspiration of medium, MTT (1 mg/ml) was then added to the cell cultures, followed by incubation for 3 h at 37° C. After cells were crystallized, MTT was removed and DMSO was added to dissolve the metabolized MTT product. The absorbance was then measured on a Wallac Victor3 1420 Multi-label counter at 540 nm.

### Western Blot Analysis.

A whole cell extract was prepared from the treated cells as described by Landis-Piwowar et al. Cancer Res. (2007) 67, 4303, Chen et al. Cancer Res. (2007) 67, 1636, and Chen et al. Biochem. Pharm. (2005) 69, 1421. Cell extracts (30 μg) were separated by an SDS-PAGE gel and transferred to nitrocellulose membranes. The membrances were blotted by specific antibodies including anti-AMPK, p-AMPK, EGFR, p-EGFR (Cell Signaling Tech., Danvers, Mass.), PARP (Enzo Life Sciences, Plymouth Meeting, Pa.), actin (Santa Cruz Biotechnology, Santa Cruz, Calif.). The membranes were visualized by enhanced chemiluminescence, as described previously by Landis-Piwowar et al. Cancer Res. (2007) 67, 4303, Chen et al. Cancer Res. (2007) 67, 1636, and Chen et al. Biochem. Pharm. (2005) 69, 1421.

### Flow Cytometry Analysis.

The treated cells were washed once with phosphate-buffered saline (PBS) and then harvested with Cell Dissociation Buffer (enzyme-free, Invitrogen, cat. #13150-016). Detached cells were washed with PBS containing 1% FCS (a wash buffer), and resuspended in the wash buffer (1,000 cells/100 μl). The cells were stained with combinations of fluorochrome-conjugated monoclonal antibodies obtained from BD Biosciences (San Diego, Calif.) against human CD44 (FITC; cat. #555478) and CD24 (PE; cat. #555428) or their respective isotype controls at concentrations recommended by the manufacturer and incubated at 4° C. in the dark for 30 to 40 min. The labeled cells were washed in the wash buffer, then fixed in PBS containing 1% paraformaldehyde, and then analyzed on a FACSVantage (BD Biosciences) (Sheridan et al. Breast Cancer Res. (2006) 8, R59).

### Example 1

### Preparation of cis-1,2,3,4-tetrahydro-naphthalene-2,3-diol (12)

To a solution of 1,4-dihydronaphthalene (500 mg, 3.84 mmol) in acetone/H₂O (3.0/1.0 mL) was added a solution of NMO in H₂O (1.43 mL, 50% wt, 6.90 mmol) and a solution of OsO₄ in 2-methyl-2-propanol (313 μL, 2.5% wt., 25 μmol). The mixture was stirred at room temperature for 16 h. Saturated Na₂SO₃ aqueous solution (10 mL) was added and stirred for an additional 15 min H₂O (10 mL) and EtOAc (30 mL) was added and stirred for 5 min. The aqueous phase was extracted with EtOAc (4×30 mL). The combined organic phase was washed with brine (20 mL) and dried over anhydrous Na₂SO₄. The solution was concentrated by rotary evaporator and vacuum drying to give the crude product which was purified by silica gel chromatography (hexane/EtOAc/CH₂Cl₂=5/1/1) to afford 521.7 mg (83%) of the title compound as a white solid. ¹H NMR (acetone-d₆, 300 MHz) δ7.13 (m, 2H), 7.09 (m, 2H), 4.11 (t, J=5.4, 2H), 3.01 (m, 4H), 2.36 (s, 2H); ¹³C NMR (acetone-d₆, 75 MHz) δ132.87, 129.11, 126.25, 69.24, 34.32.

### Preparation of Monobenzoates 14 and 19

To a solution of the corresponding benzoic acid (0.22 mmol) in dry CH₂Cl₂ (20 mL), dicyclohexylcarbodiimide (DCC, 45 mg, 0.22 mmol) was added. The resulting mixture was stirred at room temperature for 4 h. 4-Dimethylaminopyridine (DMAP, 3 mg, 0.025 mmol) was added, then a solution of diol 12 (33 mg, 0.2 mol) in CH₂Cl₂ (5 mL) was added dropwise. The mixture was stirred at room temperature overnight. Then the mixture was concentrated, ethyl acetate (1 mL) was added and cooled in fridge, the urea byproduct was filtered and the filtrate was evaporated. The resulting residue was purified by column chromatography (ethyl acetetate/n-hexane=1:3) to afford the desired compound as a pale yellow amorphous solid.

### Compound 14:

white solid (61% yield). ¹H NMR (CDCl₃, 300 MHz) δ 7.43-7.13 (m, 22H), 5.48 (brs, 1H), 5.15 (s, 2H), 5.11 (s, 4H), 4.33 (s, 1H), 3.28-3.03 (m, 4H); ¹³C NMR (CDCl₃, 75 MHz) δ 166.4, 152.7, 142.7, 137.6, 136.9, 133.3, 132.7, 129.5, 129.3, 128.9, 128.8, 128.5, 128.3, 127.8, 126.7, 126.6, 125.3, 109.4, 75.4, 73.4, 71.4, 69.8, 68.1, 35.0, 32.2.

### Compound 19

white solid (65% yield). ¹H NMR (CDCl₃, 300 MHz) δ 7.44-7.13 (m, 16H), 6.82 (s, 1H), 5.50 (brs, 1H), 5.14 (s, 1H), 5.05 (s, 4H), 4.35 (s, 1H), 3.31-3.08 (m, 4H); ¹³C NMR (CDCl₃, 75 MHz) δ 166.6, 160.0, 136.8, 133.4, 132.8, 132.3, 129.6, 129.3, 129.0, 128.5, 128.0, 126.7, 126.7, 108.9, 107.4, 73.7, 70.6, 68.0, 35.0, 32.1.

### Preparation of Dibenzoates 15 and 20

To a solution of compound 12 (33 mg, 0.2 mmol) in CH₂Cl₂ (3.0 mL) were added the corresponding benzoic acid (0.42 mmol), 4-dimethylaminopyridine (DMAP, 6 mg, 0.05 mmol) and dicyclohexylcarbodiimide (DCC, 87 mg, 0.42 mmol). The mixture was stirred at room temperature overnight. Then the mixture was concentrated, EtOAc (1 mL) was added and cooled in fridge, the urea byproduct was filtered and the filtrate was evaporated. The resulting residue was purified by column chromatography (EtOAc/n-hexane=1:6) to afford the desired compound as a white amorphous solid.

### Compound 15

white solid (59% yield). ¹H NMR (CDCl₃, 300 MHz) δ 7.43-7.22 (m, 38H), 5.72 (brs, 1H), 5.01-4.96 (m, 12H), 3.38 (dd, J=17.4 Hz, J==4.8 Hz, 1H), 3.25 (dd, J=17.4 Hz, 6.9 Hz, 1H); ¹³C NMR (CDCl₃, 75 MHz) δ 165.7, 152.7, 142.7, 137.7, 136.7, 132.6, 129.4, 128.8, 128.7, 128.5, 128.3, 128.2, 127.8, 126.9, 125.2, 109.2, 75.3, 71.2, 70.5, 32.5.

### Compound 20

white solid (71% yield). ¹H NMR (CDCl₃, 300 MHz) δ 7.42-7.18 (m, 28H), 6.74 (s, 1H), 5.72 (brs, 1H), 4.91 (m, 9H), 3.34 (m, 4H); ¹³C NMR (CDCl₃, 75 MHz) δ 165.9, 160.0, 136.6, 132.5, 132.2, 129.4, 128.8, 128.4, 127.9, 126.8, 108.6, 107.8, 70.6, 70.4, 32.3.

### General Procedures for Palladium Catalyzed Hydrogenolysis: Preparation of Compounds 5, 7, 16 and 21

To a solution of benzylated substrate (0.1 mmol) in THF/MeOH (3 mL, 1:2) was added palladium hydroxide (20 mg, 20% on carbon). The resulting mixture was stirred at room temperature until TLC showed that the reaction was completed (within 6 h). Then the reaction mixture was filtered to remove the catalyst. The filtrate was evaporated to afford product as a white solid.

### Compound 16

white solid (95% yield). ¹H NMR (CD₃OD, 300 MHz) δ 7.13-7.01 (m, 6H), 5.39 (m, 1H), 4.25 (m, 1H), 3.14-3.06 (m, 4H); ¹³C NMR (CD₃OD, 75 MHz) δ 167.1, 145.2, 138.6, 133.5, 132.8, 129.0, 128.8, 126.1, 126.0, 120.6, 109.0, 72.5, 67.4, 34.4, 32.1; HRMS m/z calculated for C₁₇H₁₆O₆Na 339.0840. found 339.0839.

### Compound 5

white solid (95% yield). ¹H NMR (CD₃OD, 300 MHz) δ 7.21-7.16 (m, 4H), 7.09 (s, 4H), 5.61 (m, 2H), 3.37-3.25 (m, 4H); ¹³C NMR (CD₃OD, 75 MHz) δ 165.3, 145.1, 138.0, 132.8, 129.0, 126.3, 120.9, 109.0, 69.7, 31.9; HRMS m/z calculated for C₂₄H₂₀O₁₀Na 491.0947. found 491.0949.

### Compound 21

white solid (95% yield). ¹H NMR (CD₃OD, 300 MHz) δ 7.14-7.07 (m, 4H), 6.92 (s, 2H), 6.44 (s, 1H), 5.43 (m, 1H), 4.27 (m, 1H), 3.17-3.10 (m, 4H); ¹³C NMR (CD₃OD, 75 MHz) δ 170.6, 166.0, 134.2, 133.2, 132.9, 129.3, 129.1, 126.3, 126.2, 108.2, 107.4, 73.2, 67.2, 35.0, 32.1; HRMS m/z calculated for C₁₇H₁₆O₅Na 323.0891. found 323.0890.

### Compound 7

white solid (95% yield). ¹H NMR (CD₃OD, 300 MHz) δ 7.16 (s, 4H), 6.88 (s, 4H), 6.46 (s, 2H), 5.66 (m, 2H), 3.31 (m, 4H); ¹³C NMR (CD₃OD, 75 MHz) δ 166.3, 158.6, 132.5, 131.9, 128.9, 126.4, 107.7, 107.3, 70.4, 31.8; HRMS m/z calculated for C₂₄H₂₀O₈Na 459.1047. found 459.1050.

### Preparation of the Acetates 6, 8, 17 and 22

To a solution of the corresponding substrate (0.1 mmol), acetic anhydride (0.5 mL) in pyridine (0.5 mL) was added at room temperature. The resulting mixture was stirred overnight. Then EtOAc (50 mL) was added and 1N HCl (1 mL) and washed with CuSO4 solution (3×10 mL), water (2×10 mL) and brine (10 mL), dried over sodium sulfate and evaporated. The residue was purified by column chromatography over silica gel (ethyl acetate/n-hexane 3:2) to afford the title product as a white solid.

### Compound 17

white solid (92% yield). ¹H NMR (CDCl₃, 300 MHz) δ 7.74 (s, 2H), 7.21-7.11 (m, 4H), 5.64 (m, 1H), 5.42 (m, 1H), 3.26-3.16 (m, 4H), 2.30 (s, 9H), 2.07 (s, 3H); ¹³C NMR (CDCl₃, 75 MHz) δ 170.9, 167.8, 166.6, 164.1, 143.7, 139.0, 132.6, 132.3, 129.3, 128.6, 126.8, 126.7, 122.5, 70.9, 69.7, 32.5, 31.7, 21.4, 20.8, 20.4; HRMS m/z calculated for C₂₅H₂₄O₁₀Na 507.1266. found 507.1262.

### Compound 6

white solid (94% yield). ¹H NMR (CDCl₃, 300 MHz) δ 7.73-7.70 (m, 4H), 7.22-7.14 (m, 4H), 5.68 (m, 2H), 3.31 (m, 4H), 2.28 (s, 18H); ¹³C NMR (CDCl₃, 75 MHz) δ 167.9, 166.6, 164.1, 143.7, 139.1, 132.2, 129.4, 128.4, 126.9, 122.6, 71.1, 32.1, 20.8, 20.4; HRMS m/z calculated for C₃₆H₃₂O₁₆Na 743.1576. found 743.1583.

### Compound 22

white solid (90% yield). ¹H NMR (CDCl₃, 300 MHz) δ 7.60 (s, 2H), 7.21-7.11 (m, 5H), 5.64 (m, 1H), 5.44 (m, 1H), 3.26-3.17 (m, 4H), 2.31 (s, 6H), 2.08 (s, 3H),; ¹³C NMR (CDCl₃, 75 MHz) δ 170.9, 169.0, 164.6, 151.1, 132.6, 132.5, 132.3, 129.3, 126.8, 126.7, 120.7, 120.6, 70.8, 69.7, 32.4, 31.8, 21.4, 21.3; HRMS m/z calculated for C₂₃H₂₂O₈Na 449.1201. found 449.1207.

### Compound 8

white solid (91% yield). NMR (CDCl₃, 300 MHz) δ 7.58 (s, 4H), 7.23-7.14 (m, 6H), 5.70 (m, 2H), 3.32 (m, 4H), 2.28 (s, 12H); ¹³C NMR (CDCl₃, 75 MHz) δ 169.0, 164.6, 151.1, 132.3, 132.2, 129.4, 126.9, 120.8, 120.6, 71.0, 32.1, 21.2; HRMS m/z calculated for C₃₂H₂₈O₁₂Na 627.1468. found 627.1473.

### Compounds 23 and 24

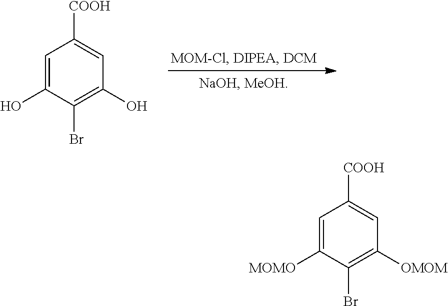

To a solution of 4-bromo-3,5-dihydroxylbenzoic acid (500 mg, 2.1 mmol) in dry DCM at 0° C. was added diisopropylethylamine (2.49 g, 18.9 mmol) dropwise, followed by methoxymethyl chloride (1.5 g, 18.9 mmol) dropwise and stirred for 48 h at room temperature. After completion of the reaction as indicated by TLC, the reaction mixture was quenched with saturated NH₄Cl solution (10 ml) and extracted twice with DCM. Removal of the solvent gave the intermediate ether ester.

To this ether ester in MeOH (80 ml) was added 15% NaOH in MeOH (80 ml) and the whole was heated at 70° C. for 3 h. The pH of the reaction was adjusted to 5-6 by addition of 6N HCl at 0° C. followed by filtration. The MOM-protected benzoic acid was obtained as a white solid, m.p. 172° C., (400 mg, 60% yield): ¹H NMR (400 MHz, CDCl₃) δ: 7.54 (s, 2H), 5.32 (s, 4H), 3.53 (s, 6H); ¹³C NMR (400 MHz, d₄-CH₃OH) δ: 167.3, 154.8, 130.7, 109.6, 108.5, 94.8, 55.2; Negative ESI MS m/z: 318 (M−1); HRMS (ESI) m/z calculated for (M-H) C₁₁H₁₂ Br O₆, 318.9811. found 318.9824.

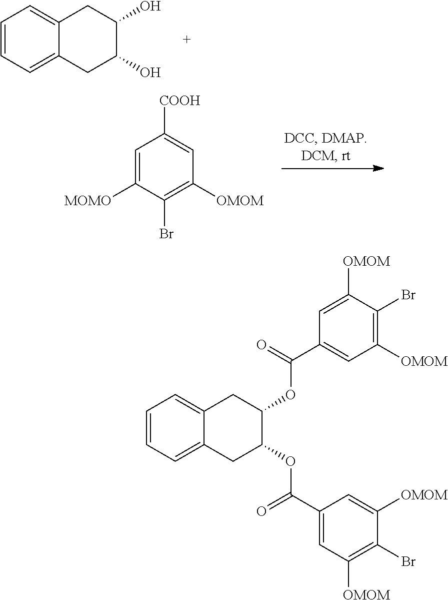

The acid (466 mg, 1.4 mmol) obtained above and dicyclohexylcarbodiimide (296 mg, 1.4 mmol) were taken in dry DCM and stirred for 1 h at room temperature. Then 4-dimethylaminopyridine (14 mg, 0.08 mmol) and the diol 12 (100 mg, 0.6 mmol), was added and stirred at room temperature. After 24 h the formed precipitate was filtered off, and purified by column chromatography (3:7, ethyl acetate:hexane) to give the product as a clear white solid, m.p. 138° C., (400 mg, 85% yield): ¹H NMR (400 MHz, CDCl₃) δ: 7.41 (s, 4H), 7.22-7.16 (m, 4H), 5.71-5.65 (m, 2H), 5.2 (s, 8H), 3.42 (s, 12H), 3.38-3.22 (m, 4H); ¹³C NMR (400 MHz, CDCl₃) δ: 165.0, 154.8, 132.0, 130.1, 129.0, 126.5, 110.2, 109.8, 95.1, 70.6, 56.4, 32.0; ESI MS m/z: 793 (M⁺ Na); HRMS (ESI) m/z calculated for (M⁺ Na) C₃₂H₃₄⁷⁹Br₂ O₁₂ Na, 791.0309. found 791.0338.

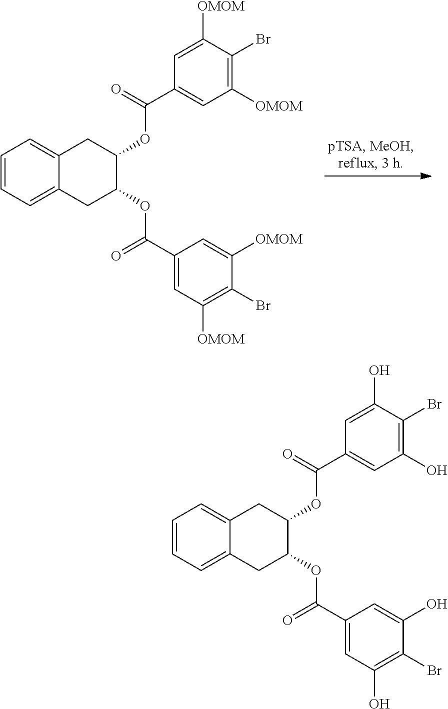

para-Toulenesulfonic acid (35 mg, 0.18 mmol) was added to a solution of the diester substrate (350 mg, 0.45 mmol) obtained above in MeOH and refluxed for 3 h. After completion of the reaction, the reaction mixture was neutralized by solid NaHCO₃, filtered and dried over NaSO₄. Purification by column chromatography gave the product 23 as white solid, m.p. 164° C., (180 mg, 65% yield): ¹H NMR (400 MHz, CDCl₃) δ: 3.4-3.2 (m, 4H), 5.75-5.6 (m, 2H), 7.11 (s, 4H), 7.20 (s, 4H), 9.13 (brs, 4H); ¹³C NMR (400 MHz, d₃-CH₃Cl) δ: 205.4, 164.8, 155.3, 132.4 130.2, 129.0, 126.4, 107.8, 70.2, 31.6; ESI MS m/z: 594 (M).

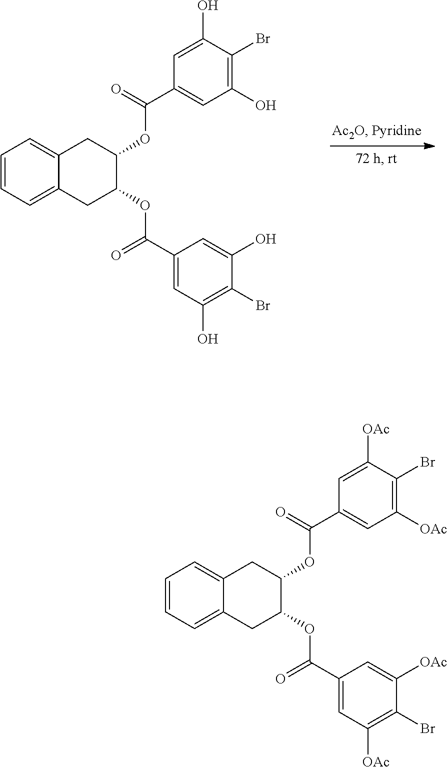 [SMILES: O=C(O[C@H]1Cc2ccccc2C[C@H]1OC(=O)c1cc(O)c(Br)c(O)c1)c1cc(O)c(Br)c(O)c1 ; CC(=O)Oc1cc(C(=O)O[C@H]2Cc3ccccc3C[C@H]2OC(=O)c2cc(OC(C)=O)c(Br)c(OC(C)=O)c2)cc(OC(C)=O)c1Br]

To compound 23 (80 mg, 0.16 mmol) was added acetic anhydride (1 ml) and pyridine (1 ml) and stirred for 3 days at room temperature. After completion of the reaction, the reaction mixture was diluted with ethyl acetate and washed with 1N HCl (10 ml), aqueous CuSO₄ solution (10 ml×3), brine (10 ml×3) and dried over Na₂SO₄. Purification by column chromatography gave compound 24 as a white solid, m.p. 158° C., (80 mg, 62% yield): ¹H NMR (400 MHz, CDCl₃) δ: 2.33 (s, 12H), 3.41-3.35 (m, 4H), 5.8-5.7 (m, 2H), 7.2 (s, 4H), 7.7 (s, 4H); ¹³C NMR (400 MHz, d₆-Acetone) δ: 205.2, 167.6, 163.6, 149.7, 132.2, 130.8, 129.0, 126.4, 121.9, 117.3, 71.0, 31.3, 19.7; ESI MS m/z: 785 (M⁺ 23); HRMS (ESI) m/z, calculated for (M⁺ Na) C₃₂H₂₆⁷⁹Br₂ O₁₂ Na, 782.9683. found 782.9714.

### Compounds 25 and 26

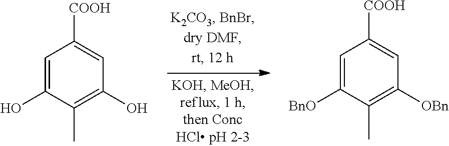 [SMILES: Cc1c(O)cc(C(=O)O)cc1O ; Cc1c(OCc2ccccc2)cc(C(=O)O)cc1OCc1ccccc1]

4-Methyl-3,5-dihydroxybenzoic acid (1 g, 5.9 mmol), K₂CO₃ (2.9 g, 21 mmol), and BnBr (3.12 g, 18.2 mmol) were dissolved in dry DMF and stirred for 12 h. Water was added to the reaction mixture and extracted with EtOAc thrice. The combined organic phase was evaporated and dissolved in 8N KOH in MeOH (50 ml) and refluxed for another 1 h. After completion of the reaction, the mixture was acidified with concentrated HCl to pH 2-3. The formed precipitate was filtered, dissolved in EtOAc and washed with water, brine and dried over NaSO₄. Removal of the solvent gave crude benzyloxybenzoic acid which on passing through a small pad of celite with ethyl acetate, gave pure product as a white solid, m.p. 220° C., (1.4 g, 70% yield): ¹H NMR (400 MHz, d₆-Acetone) δ: 7.55-7.32 (m, 12H), 5.22 (s, 4H), 2.23 (s, 3H); ¹³C NMR (500 MHz, d₆-Acetone) δ: 166.5, 157.2, 137.4, 128.9, 128.4, 127.7, 127.3, 120.3, 106.3, 70.0, 8.4; ESI MS m/z: 347 (M-H); HRMS (ESI) m/z, calculated for (M⁺ Na) C₂₂H₂₀O₄Na, 371.1253. found 371.1264.

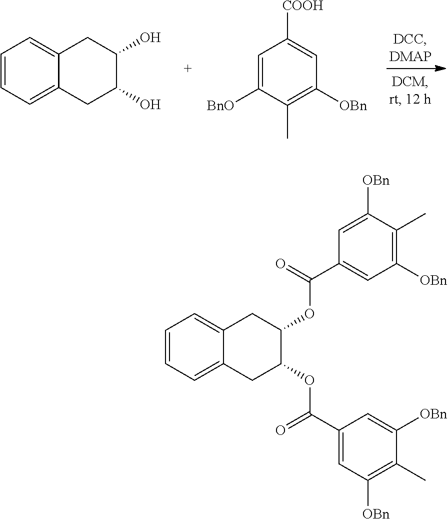 [SMILES: O[C@@H]1Cc2ccccc2C[C@@H]1O ; Cc1c(OCc2ccccc2)cc(C(=O)O)cc1OCc1ccccc1 ; Cc1c(OCc2ccccc2)cc(C(=O)O[C@H]2Cc3ccccc3C[C@H]2OC(=O)c2cc(OCc3ccccc3)c(C)c(OCc3ccccc3)c2)cc1OCc1ccccc1]

The diol 12 (50 mg, 0.3 mmol), the acid (217 mg, 0.61 mmol) obtained above, dicyclohexylcarbodiimide (263 mg, 0.61 mmol) and 4-dimethylaminopyridine (18 mg, 0.07 mmol) were dissolved in dry DCM and stirred at room temperature for 12 h. The DCM was removed and EtOAc was added and kept in the freezer for 12 h. The formed precipitate was filtered off and the crude product was purified by column chromatography to give the product as a white solid, m.p. 78° C., (120 mg, 48% yield): ¹H NMR (400 MHz, CDCl₃) δ: 2.16 (s, 6H), 3.2-3.4 (dt, 4H), 4.88 (q, 8H), 5.71 (t, 2H), 7.2-7.32 (m, 28H); ¹³C NMR (500 MHz, CDCl₃) δ: 165.8, 157.1, 136.8, 132.3, 129.0, 128.4, 128.0, 127.8, 127.2, 126.6, 121.6, 106.2, 70.1, 70.0, 32.1, 9.1; ESI MS m/z: 847 (M+Na); HRMS (ESI) m/z, calculated for (M⁺ Na) C₅₄H₄₈O₈Na, 847.3241. found 847.3254.

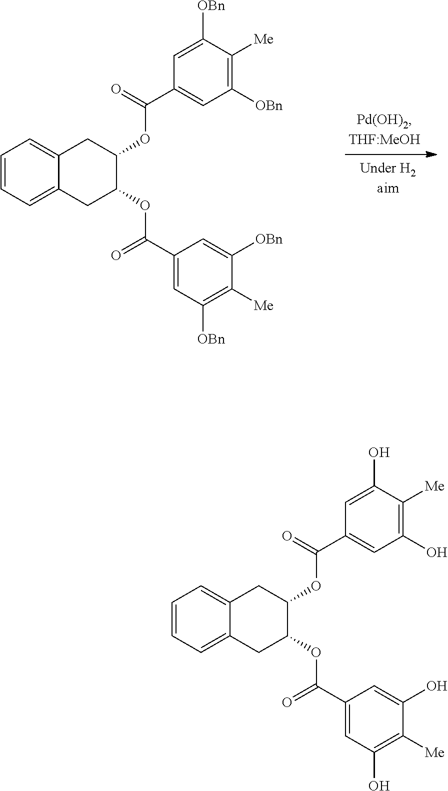 [SMILES: Cc1c(OCc2ccccc2)cc(C(=O)O[C@H]2Cc3ccccc3C[C@H]2OC(=O)c2cc(OCc3ccccc3)c(C)c(OCc3ccccc3)c2)cc1OCc1ccccc1 ; Cc1c(O)cc(C(=O)O[C@H]2Cc3ccccc3C[C@H]2OC(=O)c2cc(O)c(C)c(O)c2)cc1O]

Pd(OH)₂ (60 mg, 20% wt) was added to a solution of substrate (300 mg, 0.3 mmol) in THF:MeOH (1:2, 6 ml), and the reaction mixture was stirred at room temperature for 3 h. After complete conversion of the starting material into product, the Pd(OH)₂ was filtered off and the solvent was removed to give the product 25 as a white solid, m.p. 142° C., (166 mg, 99% yield): ¹H NMR (500 MHz, d₆-Acetone) δ: 8.42 (s, 4H), 7.14 (s, 4H), 7.0 (s, 4H), 5.6 (t, 2H), 3.38-3.22 (m, 4H), 2.07 (s, 6H); ¹³C NMR (500 MHz, d₆-Acetone) δ: 165.4, 156.1, 132.6, 128.9, 128.2, 126.3, 116.7, 107.5, 69.8, 31.7, 8.0; ESI MS m/z: 463 (M-H); HRMS (ESI) m/z, calculated for (M⁺ Na) C₂₆H₂₄O₈Na, 487.1363. found 487.1370.

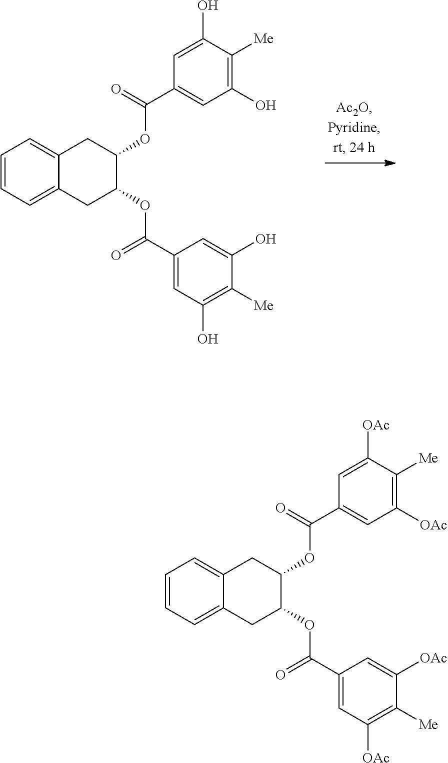 [SMILES: Cc1c(O)cc(C(=O)O[C@H]2Cc3ccccc3C[C@H]2OC(=O)c2cc(O)c(C)c(O)c2)cc1O ; CC(=O)Oc1cc(C(=O)O[C@H]2Cc3ccccc3C[C@H]2OC(=O)c2cc(OC(C)=O)c(C)c(OC(C)=O)c2)cc(OC(C)=O)c1C]

To compound 25 (50 mg, 0.1 mmol) was added acetic anhydride (1 ml) and pyridine (1 ml) and stirred for 24 h at room temperature. After completion of the reaction, the reaction mixture was diluted with ethyl acetate and washed with 1N HCl (10 ml), CuSO₄, (10 ml×3), brine (10 ml×3) and dried over Na₂SO₄. Purification by column chromatography gave compound 26 as a white solid, m.p. 110° C., (60 mg, 88% yield): ¹H NMR (400 MHz, CDCl₃) δ: 7.53 (s, 4H), 7.25-7.15 (m, 4H), 5.66 (t, 2H), 3.4-3.29 (m, 4H). 2.31 (s, 12H), 2.01 (s, 6H); ¹³C NMR (500 MHz, d₃-CH₃C1) δ: 168.6, 164.4, 149.8, 132.1, 129.5, 129.1, 128.9, 126.5, 121.0, 70.6, 31.9, 20.6, 10.4; ESI MS m/z: 655 (M+Na); HRMS (ESI) m/z, calculated for (M⁺ Na) C₃₄H₃₂O₁₂Na, 655.1786. found 655.1800.

### Compounds 27 and 28

### 4-Benzyloxybenzoic Acid

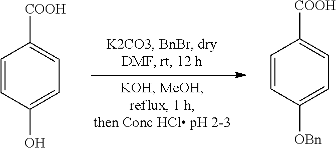 [SMILES: O=C(O)c1ccc(O)cc1 ; O=C(O)c1ccc(OCc2ccccc2)cc1]

The 4-hydroxybenzoic acid (2 g, 14.4 mmol), K₂CO₃ (4.1 g, 30 mmol), and BnBr (5.4 g, 30 mmol) were dissolved in dry DMF and stirred for 12 h. Water was added to the reaction mixture and extracted with EtOAc thrice. The combined organic phase was evaporated and dissolved in 8N KOH in MeOH (50 mL) and refluxed for another 1 h. After completion of the reaction, the mixture was acidified with concentrated HCl to pH 2-3. The formed precipitate was filtered, dissolved in EtOAc and washed with water, brine and dried over Na₂SO₄. Removal of the solvent gave crude 4-benzyloxy-benzoic acid which on passing through a small pad of celite with ethyl acetate gave pure product as a white solid, m.p. 189-191° C. (2.4 g, 72% yield); ¹H NMR (400 MHz, d6-Acetone) δ: 11.0 (brs, 1H), 8.01 (s, 2H), 7.51-7.34 (m, 5H), 7.12 (d, 2H), 5.22 (s, 2H).

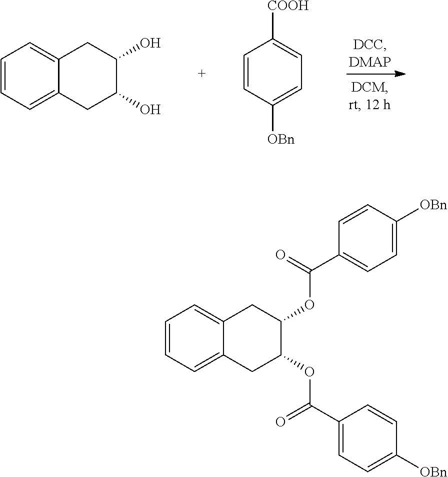 [SMILES: O[C@@H]1Cc2ccccc2C[C@@H]1O ; O=C(O)c1ccc(OCc2ccccc2)cc1 ; O=C(O[C@H]1Cc2ccccc2C[C@H]1OC(=O)c1ccc(OCc2ccccc2)cc1)c1ccc(OCc2ccccc2)cc1]

Diol 12 (50 mg, 0.3 mmol), 4-benzyloxybenzoic acid (291 mg, 0.61 mmol), dicyclohexylcarbodiimide (263 mg, 0.61 mmol) and 4-dimethylaminopyridine (9 mg, 0.07 mmol) were dissolved in dry DCM and stirred at room temperature for 12 h. The DCM was removed and EtOAc was added and kept in the freezer for 12 h. The formed precipitate was filtered off and the crude product was purified by column chromatography to give the product as a white solid, m.p. 134° C., (115 mg, 64% yield): ¹H NMR (400 MHz, CDCl₃) δ: 3.23-3.4 (m, 4H), 5.09 (2, 4H), 5.67 (t, 2H), 6.93 (d, 4H), 7.14-7.41 (m, 14H), 7.92 (d, 4H); ¹³C NMR (500 MHz, CDCl₃) δ: 165.6, 162.5, 136.1, 132.5, 131.7, 129.1, 128.6, 128.2, 127.4, 126.4, 122.7, 114.4, 70.0, 69.9, 32.2; ESI MS m/z: 607 (M+Na); HRMS (ESI) m/z, calculated for (M⁺ Na) C₃₈H₃₂O₆Na, 607.2091. found 607.2101.

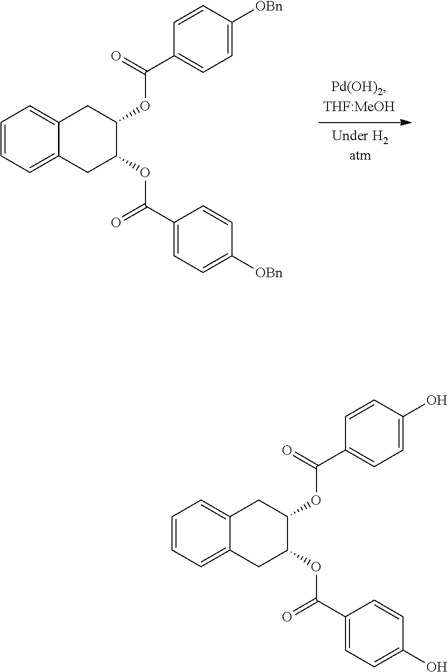 [SMILES: O=C(O[C@H]1Cc2ccccc2C[C@H]1OC(=O)c1ccc(OCc2ccccc2)cc1)c1ccc(OCc2ccccc2)cc1 ; O=C(O[C@H]1Cc2ccccc2C[C@H]1OC(=O)c1ccc(O)cc1)c1ccc(O)cc1]

Pd(OH)₂ (40 mg, 20% wt) was added to a solution of the substrate (230 mg, 0.3 mmol) obtained above in THF:MeOH (1:2, 6 ml), and the reaction mixture was stirred at room temperature for 3 h. After complete conversion of the starting material into product, the Pd(OH)₂ was filtered off and the solvent was removed to give compound 27 as a white solid, m.p. 142° C., (155 mg, 98% yield): ¹H NMR (400 MHz, d₆-Acetone) δ: 7.82 (d, 4H), 7.22-7.17 (m, 4H), 6.84 (d, 4H), 5.65 (t, 2H), 3.32 (dt, 4H); ¹³C NMR (500 MHz, d₆-Acetone) δ: 165.2, 162.6, 132.8, 131.6, 129, 126.2, 120.9, 115.2, 69.6, 31.9; ESI MS m/z: 403 (M-H); HRMS (ESI) m/z, calculated for (M⁺ Na) C₂₄H₂₀O₆Na, 427.1152. found 427.1164.

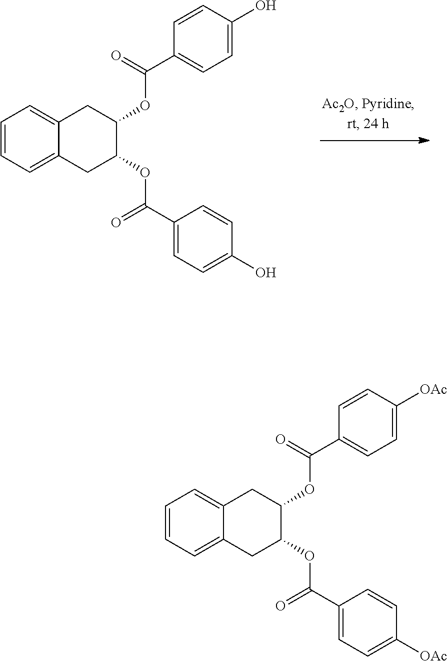 [SMILES: O=C(O[C@H]1Cc2ccccc2C[C@H]1OC(=O)c1ccc(O)cc1)c1ccc(O)cc1 ; CC(=O)Oc1ccc(C(=O)O[C@H]2Cc3ccccc3C[C@H]2OC(=O)c2ccc(OC(C)=O)cc2)cc1]

To compound 27 (40 mg, 0.09 mmol) was added acetic anhydride (1 ml) and pyridine (1 ml) and stirred for 24 h at room temperature. After completion of the reaction, the reaction mixture was diluted with ethyl acetate and washed with 1N HCl (10 ml), CuSO₄, (10 ml×3), brine (10 ml×3) and dried over Na₂SO₄. Purification by column chromatography gave compound 28 as white solid, m.p. 150° C., (42 mg, 88% yield): ¹H NMR (400 MHz, CDCl₃) δ: 8.01 (d, 4H), 7.2-7.11 (m, 8H), 5.7 (t, 2H), 3.33 (4H), 2.3 (s, 6H); ¹³C NMR (500 MHz, CDCl₃) δ: 168.8, 165.1, 154.4, 132.2, 131.2, 129.1, 127.6, 126.5, 121.6, 70.3, 32.1, 21.1; ESI MS m/z: 511 (M+Na); HRMS (ESI) m/z, calculated for (M⁺ Na) C₂₈H₂₄O₈Na, 511.1387. found 511.1375.

### Compounds 29, 30 and 31

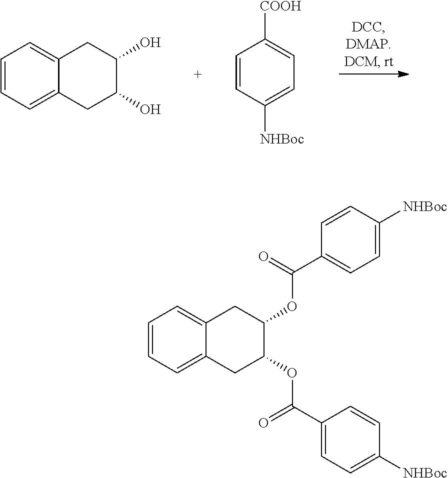 [SMILES: O[C@@H]1Cc2ccccc2C[C@@H]1O ; CC(C)(C)OC(=O)Nc1ccc(C(=O)O)cc1 ; CC(C)(C)OC(=O)Nc1ccc(C(=O)O[C@H]2Cc3ccccc3C[C@H]2OC(=O)c2ccc(NC(=O)OC(C)(C)C)cc2)cc1]

The N-boc protected acid (45 mg, 0.21 mmol) and dicyclohexylcarbodiimide (42 mg, 0.21 mmol) were taken in dry DCM and stirred for 2 h at room temperature. 4-Dimethylaminopyridine (3 mg, 0.03 mmol) and the diol 12 (20 mg, 0.12 mmol) were added and stirred at room temperature. After 24 h the formed precipitate was filtered off, and purified by column chromatography (1.5:8.5, ethyl acetate:hexane) to give compound 29 as a white solid, m.p. 110° C., (45 mg, 60% yield): ¹H NMR (400 MHz, CDCl₃) δ: 1.47 (s, 18H), 3.4-3.3.35 (m, 4H), 5.8-5.65 (m, 2H), 7.2 (s, 4H), 7.63 (d, 4H), 7.90 (d, 4H). 8.78 (brs, 2H); ¹³C NMR (400 MHz, d6-Acetone) δ: 205.3, 165.0, 152.4, 144.2, 132.7, 130.5, 129.0, 126.3, 123.7, 117.2, 117.1, 79.8, 69.8, 31.8, 27.5; ESI MS m/z: 625 (M+Na); HRMS (ESI) m/z, calculated for (M⁺ Na) C₃₄H₃₈N₂O₈Na, 625.2520. found 625.2543.

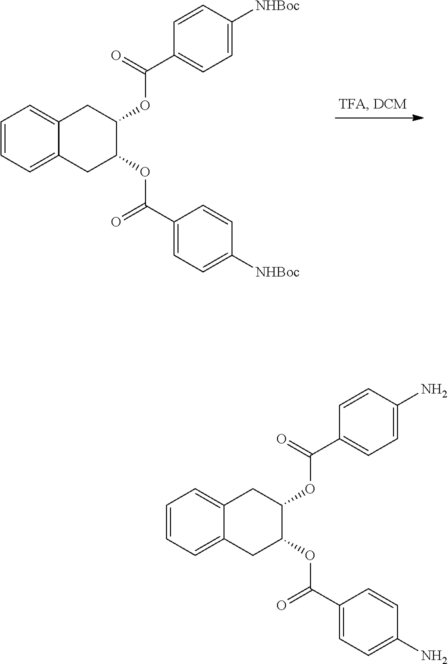 [SMILES: CC(C)(C)OC(=O)Nc1ccc(C(=O)O[C@H]2Cc3ccccc3C[C@H]2OC(=O)c2ccc(NC(=O)OC(C)(C)C)cc2)cc1 ; Nc1ccc(C(=O)O[C@H]2Cc3ccccc3C[C@H]2OC(=O)c2ccc(N)cc2)cc1]

Compound 29 (130 mg, 0.2 mmol) was dissolved in DCM and a little excess of trifluoroacetic acid (246 mg, 2 mmol) was added and stirred at room temperature overnight. The excess of trifluoroacetic acid was removed and the crude product was purified by column chromatography to give compound 30 as a white solid, m.p. 94° C., (80 mg, 92% yield): ¹H NMR (300 MHz, d₆-Acetone) δ: 3.29-3.36 (m, 4H), 5.58-5.62 (m 2H), 6.62 (d, 4H), 6.76 (d, 1/2H), 7.17 (s, 4H), 7.69 (d, 4H), 7.92 (d, 1/2H); ¹³C NMR (400 MHz, d6-Acetone) δ: 205.3, 165.4, 132.9, 131.3, 128.9, 126.1, 117.6, 112.8, 69.2, 32.0; ESI MS m/z: 425 (M+Na); HRMS (ESI) m/z, calculated for (M⁺ Na) C₂₄H₂₂N₂O₄Na, 425.1471. found 425.1485.

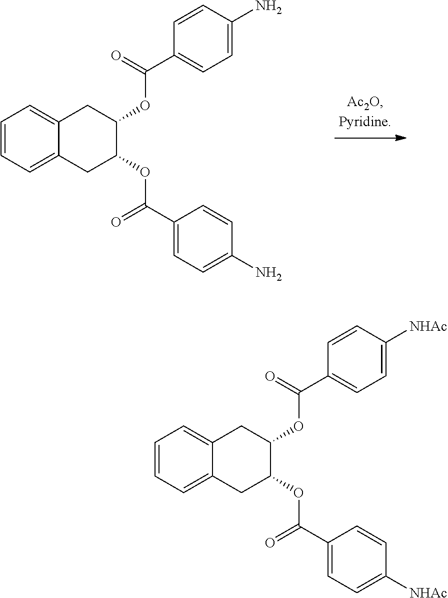 [SMILES: Nc1ccc(C(=O)O[C@H]2Cc3ccccc3C[C@H]2OC(=O)c2ccc(N)cc2)cc1 ; CC(=O)Nc1ccc(C(=O)O[C@H]2Cc3ccccc3C[C@H]2OC(=O)c2ccc(NC(C)=O)cc2)cc1]

Compound 30 (40 mg, 0.09 mmol), acetic anhydride (0.5 ml) and pyridine (0.5 ml) were stirred at room temperature for 24 h. After completion of the reaction, ethyl acetate was added and stirred for 5 min, then 1N HCL (1 ml) was added and stirred for another 5 min. The solution was washed with CuSO₄ solution (2×10 ml), water (2×10 ml), brine (2×10 ml), dried over Na₂SO₄ and purified by column chromatography to give compound 31 as a white solid, m.p. 128° C., (40 mg, 82% yield): ¹H NMR (300 MHz, d₂-DCM) δ: 2.14 (s, 6H), 3.34-3.24 (m, 4H), 5.5.72-5.62 (m, 2H), 7.18 (s, 4H), 7.55 (d, 4H), 7.90 (d, 6H); ¹³C NMR (300 MHz, d₂-DCM) δ: 169.7, 165.5, 142.9, 132.4, 130.6, 129.0, 126.3, 124.9, 118.7, 70.0, 31.9, 24.0; ESI MS m/z: 509 (M+Na); HRMS (ESI) m/z, calculated for (M⁺ Na) C₂₈H₂₆N₂O₆Na, 509.1683. found 509.1702.

### Compound 32

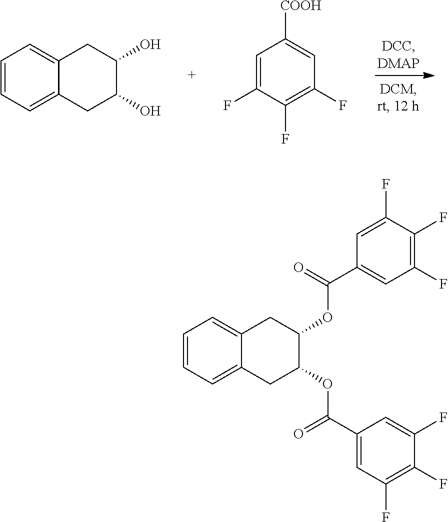 [SMILES: O[C@@H]1Cc2ccccc2C[C@@H]1O ; O=C(O)c1cc(F)c(F)c(F)c1 ; O=C(O[C@H]1Cc2ccccc2C[C@H]1OC(=O)c1cc(F)c(F)c(F)c1)c1cc(F)c(F)c(F)c1]

The diol 12 (50 mg, 0.3 mmol), 3,4,5-trifluorobenzoic acid (109 mg, 0.61 mmol), DCC (128 mg, 0.61 mmol) and DMAP (7 mg, 0.07 mmol) were dissolved in dry DCM and stirred at room temperature for 12 h. The DCM was removed and EtOAc was added and kept in the freezer for 12 h. The formed precipitate was filtered off and the crude product was purified by column chromatography to give the product as a white solid, m.p. 118-120° C. (80 mg, 54% yield). ¹H NMR (400 MHz, CDCl₃) δ: 3.4-3.24 (m, 4H), 5.70 (t, 2H), 7.30-7.18 (m, 14H), 7.65-7.55 (m, 4H); ¹³C NMR (300 MHz, CDCl₃) δ: 31.8, 71.0, 77.4, 114.1, 114.2, 114.3, 114.4, 125.6, 125.7, 126.8, 129.1, 131.5, 141.5, 141.7, 144.8, 145.0, 145.2, 149.2, 149.3, 149.4, 152.5, 152.6, 152.7, 152.7, 163.2.

### Compound 33

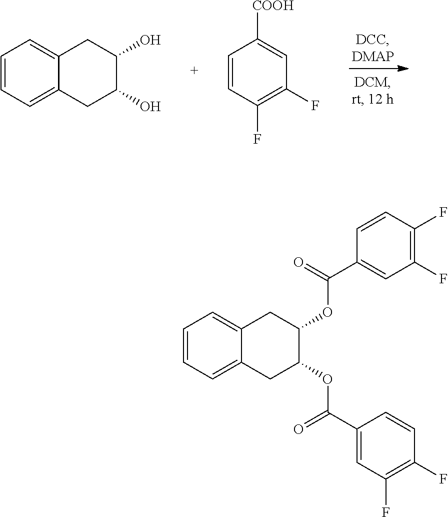 [SMILES: O[C@@H]1Cc2ccccc2C[C@@H]1O ; O=C(O)c1ccc(F)c(F)c1 ; O=C(O[C@H]1Cc2ccccc2C[C@H]1OC(=O)c1ccc(F)c(F)c1)c1ccc(F)c(F)c1]

The diol 12 (50 mg, 0.3 mmol), 3,4-difluorobenzoic acid (101 mg, 0.61 mmol), DCC (131 mg, 0.61 mmol) and DMAP (7 mg, 0.07 mmol) were dissolved in dry DCM and stirred at room temperature for 12 h. The DCM was removed and EtOAc was added and kept in the freezer for 12 h. The formed precipitate was filtered off and the crude product was purified by column chromatography to give the product as a white solid, m.p. 102-104° C. (91 mg, 66% yield). ¹H NMR (400 MHz, CDCl₃) δ: 3.4-3.3 (m, 4H), 5.71 (t, 2H), 7.28-7.15 (m, 6H), 7.80-7.70 (m, 4H); ¹³C NMR (500 MHz, CDCl₃) δ: 164.0, 164.0, 154.8, 154.7, 152.7, 152.6, 151.1, 151.0, 149.0, 131.8, 129.1, 126.9, 126.9, 126.9, 126.8, 126.7, 126.6, 126.6, 126.6, 126.6, 119.0, 118.9, 118.9, 117.5, 117.3, 70.6, 31.9; HRMS (ESI) m/z, calculated for (M⁺ Na) C₂₄H₁₆O₄F₄Na, 467.0876. found 467.0885.

### Compound 34

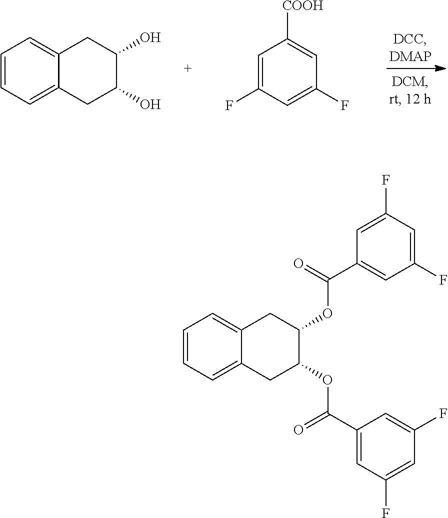 [SMILES: O[C@@H]1Cc2ccccc2C[C@@H]1O ; O=C(O)c1cc(F)cc(F)c1 ; O=C(O[C@H]1Cc2ccccc2C[C@H]1OC(=O)c1cc(F)cc(F)c1)c1cc(F)cc(F)c1]

The diol 12 (50 mg, 0.3 mmol), 3,5-difluorobenzoic acid (101 mg, 0.61 mmol), DCC (131 mg, 0.61 mmol) and DMAP (7 mg, 0.07 mmol) were dissolved in dry DCM and stirred at room temperature for 12 h. The DCM was removed and EtOAc was added and kept in the freezer for 12 h. The formed precipitate was filtered off and the crude product was purified by column chromatography to give the product as a white solid, m.p. 136-138° C. (91 mg, 66% yield). ¹H NMR (400 MHz, CDCl₃) δ: 3.4-3.30 (m, 4H), 5.72 (t, 2H), 7.02-6.98 (m, 2H) 7.30-7.18 (m, 4H), 7.47-7.40 (m, 4H); ³³C NMR (500 MHz, CDCl₃) δ: 164.4, 164.3, 163.8, 163.8, 163.7, 161.1, 161.0, 133.1, 133.0, 132.9, 131.7, 129.1, 126.7, 112.8, 112.7, 112.6, 112.5, 109.0, 108.7, 108.3, 70.9, 31.8; HRMS (ESI) m/z, calculated for (M⁺ Na) C₂₄H₁₈O₄F₄Na, 467.0876. found 467.0886.

### Compound 46

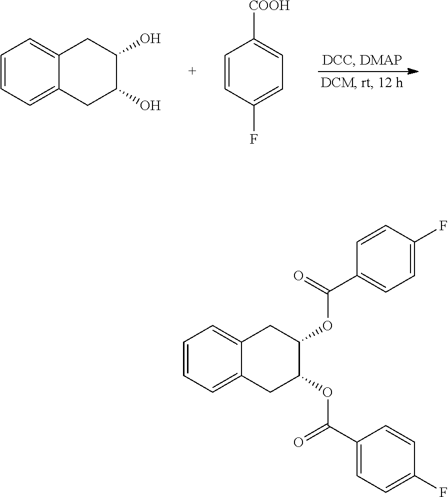 [SMILES: O[C@@H]1Cc2ccccc2C[C@@H]1O ; O=C(O)c1ccc(F)cc1 ; O=C(O[C@H]1Cc2ccccc2C[C@H]1OC(=O)c1ccc(F)cc1)c1ccc(F)cc1]

The diol 12 (50 mg, 0.3 mmol), 4-fluorobenzoic acid (87 mg, 0.61 mmol), DCC (128 mg, 0.61 mmol) and DMAP (7 mg, 0.07 mmol) were dissolved in dry DCM and stirred at room temperature for 12 h. The DCM was removed and EtOAc was added and kept in the freezer for 12 h. The formed precipitate was filtered off and the crude product was purified by column chromatography to give pure compound 46 as a white solid, m.p. 108-110° C. (30 mg, 24% yield). ¹H NMR (400 MHz, CDCl3) δ: 7.99-7.96 (m, 4H), 7.25-7.04 (m, 8H), 5.71 (m, 2H), 3.4-3.33 (m, 4H); ¹³C NMR (500 MHz, CDCl3) δ: 166.8, 164.9, 164.8, 132.2, 132.1, 129.1, 126.5, 126.2, 126.2, 115.6, 115.4, 70.3, 32.0; HRMS (ESI) m/z calculated for (M⁺Na) C₂₄H₁₈O₄F₂Na, 431.10654. found 431.10680.

### Compound 47

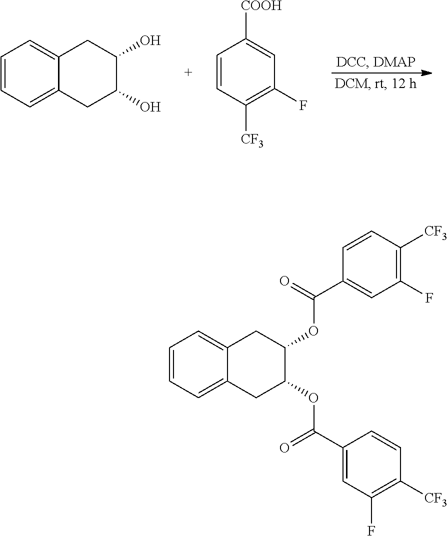 [SMILES: O[C@@H]1Cc2ccccc2C[C@@H]1O ; O=C(O)c1ccc(C(F)(F)F)c(F)c1 ; O=C(O[C@H]1Cc2ccccc2C[C@H]1OC(=O)c1ccc(C(F)(F)F)c(F)c1)c1ccc(C(F)(F)F)c(F)c1]

The diol 12 (50 mg, 0.3 mmol), 3-fluoro-4-trifluoromethylbenzoic acid (133 mg, 0.61 mmol), DCC (128 mg, 0.61 mmol) and DMAP (7 mg, 0.07 mmol) were dissolved in dry DCM and stirred at room temperature for 12 h. The DCM was removed and EtOAc was added and kept in the freezer for 12 h. The formed precipitate was filtered off and the crude product was purified by column chromatography to give compound 47 as a white solid. m.p. 91-93° C. (90 mg, 54% yield). ¹H NMR (400 MHz, CDCl₃) δ: 7.85-7.65 (m, 6H), 7.25-7.18 (m, 4H), 5.79-5.77 (m, 2H), 3.4-3.33 (m, 4H); ¹³C NMR (500 MHz, CDCl₃) δ: 163.6, 163.6, 160.5, 160.5, 158.5, 158.5, 135.5, 135.5, 135.4, 131.6, 129.1, 127.5, 127.5, 127.5, 127.4, 126.8, 125.2, 125.1, 123.0, 122.7, 122.6, 122.4, 122.3, 120.9, 120.9, 118.1, 117.9, 71.1, 31.8; MS HRMS (ESI) m/z calculated for (M⁺Na) C₂₆H₁₆O₄F₈Na, 567.0813. found 567.0827.

### Compound 48

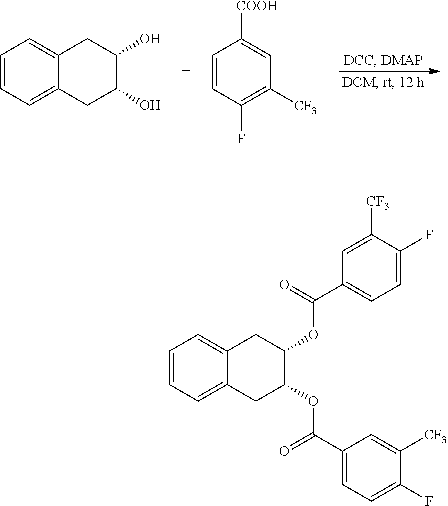 [SMILES: O[C@@H]1Cc2ccccc2C[C@@H]1O ; O=C(O)c1ccc(F)c(C(F)(F)F)c1 ; O=C(O[C@H]1Cc2ccccc2C[C@H]1OC(=O)c1ccc(F)c(C(F)(F)F)c1)c1ccc(F)c(C(F)(F)F)c1]

The diol 12 (50 mg, 0.3 mmol), 3-trifluoromethyl-4-fluorobenzoic acid (130 mg, 0.61 mmol), DCC (128 mg, 0.61 mmol) and DMAP (7 mg, 0.07 mmol) were dissolved in dry DCM and stirred at room temperature for 12 h. The DCM was removed and EtOAc was added and kept in the freezer for 12 h. The formed precipitate was filtered off and the crude product was purified by column chromatography to give compound 48 as a white solid. m.p. 100-102° C. (80 mg, 48% yield). ¹H NMR (400 MHz, CDCl₃) δ: 8.23-8.16 (m, 4H), 7.26-7.18 (m 6H), 5.77-5.76 (m, 2H), 3.4-3.34 (m 4H); ¹³C NMR (500 MHz, CDCl₃) δ: 163.8, 163.7, 163.7, 161.6, 161.6, 135.5, 135.5, 131.7, 129.3, 129.3, 129.2, 129.1, 126.8, 126.4, 126.4, 122.9, 120.8, 119.1, 119.0, 118.8, 118.7, 117.4, 117.2, 70.8, 31.9; HRMS (ESI) m/z calculated for (M⁺Na) C₂₆H₁₆O₄F₈Na, 567.0813. found 567.0819.

### 4-Benzyloxy-3,5-dichlorobenzoic acid

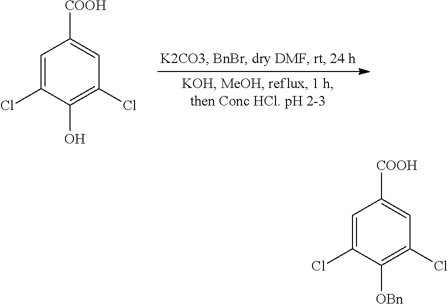 [SMILES: O=C(O)c1cc(Cl)c(O)c(Cl)c1 ; O=C(O)c1cc(Cl)c(OCc2ccccc2)c(Cl)c1]

The 4-hydroxy-3,5-dichloro-benzoic acid (2 g, 9.6 mmol), K₂CO₃ (4.1 g, 20.2 mmol), and BnBr (5.4 g, 20.1 mmol) were dissolved in dry DMF and stirred for 24 h. Water was added to the reaction mixture and extracted with EtOAc thrice. The combined organic phase was evaporated and dissolved in 8N KOH in MeOH (50 mL) and refluxed for another 1 h. After completion of the reaction, the mixture was acidified with concentrated HCl to pH 2-3. The formed precipitate was filtered, dissolved in EtOAc and washed with water, brine and dried over Na₂SO₄. Removal of the solvent gave product as light yellow color solid (2.1 g, 74% yield). ¹H NMR (400 MHz, d6-Acetone) δ: 11.8 (brs, 1H), 8.01 (s, 2H), 7.59 (d, 2H), 7.42 (d, 3H), 5.1 (s, 2H); ¹³C NMR (400 MHz, d6-Acetone) δ: 164.1, 136.1, 130.2, 129.6, 128.6, 128.5, 128.4, 128.2, 75.0.

### 1,4-Dihydronaphth-2,3-diyl di-4-benzyloxy-3,5-dichlorobenzoate

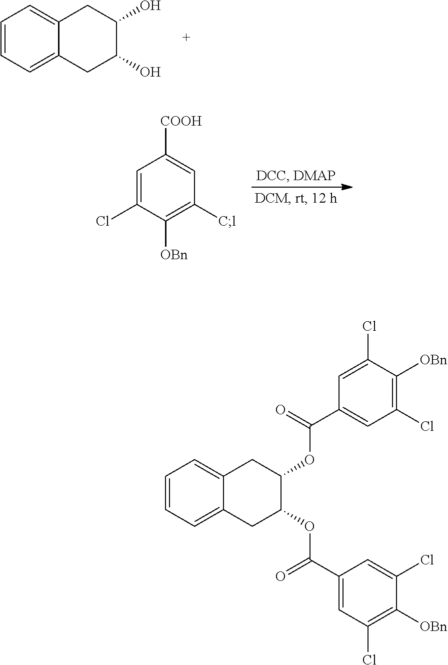

The diol 12 (50 mg, 0.3 mmol), 4-benzyloxy-3,5-dichloro-benzoic acid (185 mg, 0.61 mmol), DCC (128 mg, 0.61 mmol) and DMAP (7 mg, 0.07 mmol) were dissolved in dry DCM and stirred at room temperature for 24 h. The DCM was removed and EtOAc was added and kept in the freezer for 12 h. The formed precipitate was filtered off and the crude product was purified by column chromatography to give the product as a white solid, m.p. 134-136° C. (125 mg, 58% yield). ¹H NMR (400 MHz, CDCl₃) δ: 7.9 (s, 4H), 7.54-7.52 (m, 4H), 7.40-7.37 (m, 6H), 7.25-7.19 (m, 4H), 5.71 (t, 2H), 5.09 (s, 4H), 3.33 (d, 4H); ¹³C NMR (500 MHz, CDCl₃) δ: 163.4, 155.0, 135.6, 131.7, 130.3, 130.0, 129.1, 128.6, 128.5, 128.5, 127.1, 126.7, 75.2, 70.8, 31.8.

### Compound 49

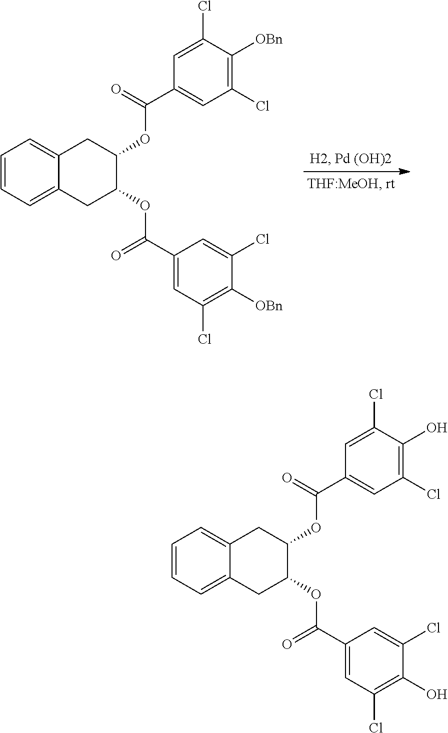 [SMILES: O=C(O[C@H]1Cc2ccccc2C[C@H]1OC(=O)c1cc(Cl)c(OCc2ccccc2)c(Cl)c1)c1cc(Cl)c(OCc2ccccc2)c(Cl)c1 ; O=C(O[C@H]1Cc2ccccc2C[C@H]1OC(=O)c1cc(Cl)c(O)c(Cl)c1)c1cc(Cl)c(O)c(Cl)c1]

The dibenzoate substrate (120 mg, 0.16 mmol), was dissolved in THF:MeOH (1:2) and Pd(OH)₂ (20 mg) was added and stirred at room temperature under H₂ atm for 3 h. After completion of the reaction as indicated by TLC, the reaction mixture was filtered through a small pad of celite and the solvents were removed under reduced pressure and purified by column chromatography to give compound 49 as a white solid, m.p. 98-100° C. (78 mg, 86% yield). ¹H NMR (400 MHz, d6-Acetone) δ: 9.8 (brs, 1H) 7.88 (s, 4H), 7.21 (s, 4H), 5.78-5.72 (m, 2H), 3.41-3.35 (m, 4H); ¹³C NMR (400 MHz, d6-Acetone) δ: 163.3, 153.3, 132.3, 129.7, 128.9, 126.4, 123.0, 121.8, 70.6, 31.5.

### Compound 50

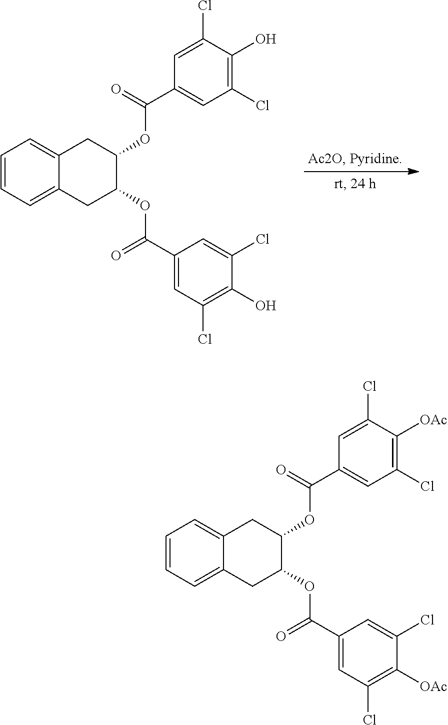 [SMILES: O=C(O[C@H]1Cc2ccccc2C[C@H]1OC(=O)c1cc(Cl)c(O)c(Cl)c1)c1cc(Cl)c(O)c(Cl)c1 ; CC(=O)Oc1c(Cl)cc(C(=O)O[C@H]2Cc3ccccc3C[C@H]2OC(=O)c2cc(Cl)c(OC(C)=O)c(Cl)c2)cc1Cl]

To the substrate (50 mg, 0.09 mmol) was added Ac₂O (1 mL), and pyridine (1 mL) and stirred for 24 h at room temperature. After completion of the reaction, the reaction mixture was diluted with ethyl acetate and washed with 1N HCl (10 mL), CuSO₄, (10 mL×3), brine (10 mL×3), dried over Na₂SO₄, and purified by column chromatography to give compound 50 as a white solid, m.p. 95-97° C. (48 mg, 80% yield). ¹H NMR (400 MHz, CDCl₃) δ: 7.93 (s, 4H), 7.3-7.18 (m, 4H), 5.78-5.71 (m, 2H), 3.38-3.3 (m, 4H), 2.4 (s, 6H); ¹³C NMR (500 MHz, CDCl₃) δ: 166.6, 163.3, 147.8, 131.6, 129.8, 129.4, 129.1, 129.1, 126.8, 71.0, 31.8, 21.0.

### Compound 51

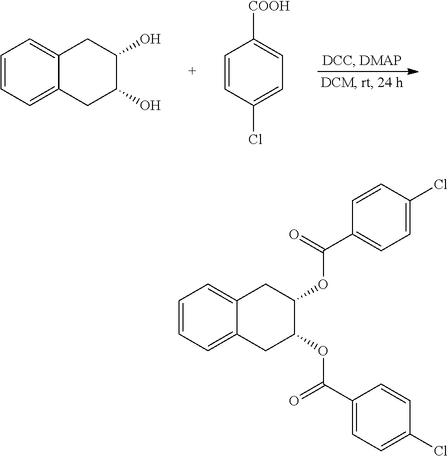 [SMILES: O[C@@H]1Cc2ccccc2C[C@@H]1O ; O=C(O)c1ccc(Cl)cc1 ; O=C(O[C@H]1Cc2ccccc2C[C@H]1OC(=O)c1ccc(Cl)cc1)c1ccc(Cl)cc1]

The diol 12 (50 mg, 0.3 mmol), 4-chlorobenzoic acid (97 mg, 0.61 mmol), DCC (128 mg, 0.61 mmol) and DMAP (7 mg, 0.07 mmol) were dissolved in dry DCM and stirred at room temperature for 24 h. The DCM was removed and EtOAc was added and kept in the freezer for 12 h. The formed precipitate was filtered off and the crude product was purified by column chromatography to give compound 51 as a white solid, m.p. 145-147° C. (102 mg, 75% yield). ¹H NMR (400 MHz, CDCl₃) δ: 7.9 (d, 4H), 7.36 (d, 4H), 7.25-7.15 (m, 4H), 5.72 (t, 2H), 3.33 (d, 4H); ¹³C NMR (500 MHz, CDCl₃) δ: 165.1, 139.6, 132.1, 131.0, 129.1, 128.7, 128.4, 126.6, 70.3, 32.0.

### Compound 52

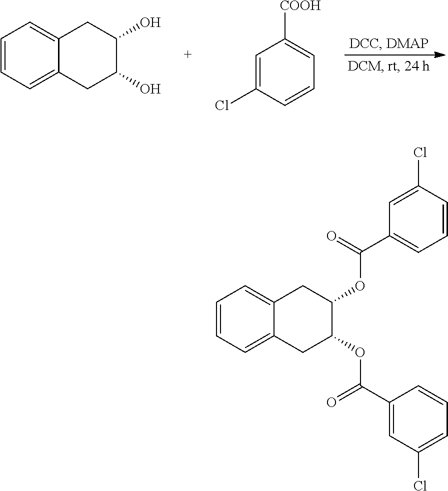 [SMILES: O[C@@H]1Cc2ccccc2C[C@@H]1O ; O=C(O)c1cccc(Cl)c1 ; O=C(O[C@H]1Cc2ccccc2C[C@H]1OC(=O)c1cccc(Cl)c1)c1cccc(Cl)c1]

The diol 12 (50 mg, 0.3 mmol), 3-chlorobenzoic acid (97 mg, 0.61 mmol), DCC (128 mg, 0.61 mmol) and DMAP (7 mg, 0.07 mmol) were dissolved in dry DCM and stirred at room temperature for 24 h. The DCM was removed and EtOAc was added and kept in the freezer for 12 h. The formed precipitate was filtered off and the crude product was purified by column chromatography to give compound 52 as a white solid, m.p. 153-155° C. (98 mg, 73% yield). ¹H NMR (500 MHz, CDCl₃) δ: 7.95 (2, 2H), 7.90 (d, 2H), 7.50 (d, 2H), 7.35 (t, 2H), 7.22-7.18 (m, 4H), 5.74 (t, 2H), 3.41-3.32 (m, 4H); ¹³C NMR (500 MHz, CDCl₃) δ: 164.8, 134.5, 133.2, 132.0, 132.0, 131.7, 129.7, 129.7, 129.1, 127.8, 126.8, 70.5, 31.9.

### 3-Benzyloxy-5-bromobenzoic acid

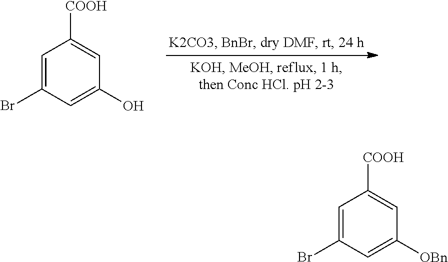 [SMILES: O=C(O)c1cc(O)cc(Br)c1 ; O=C(O)c1cc(Br)cc(OCc2ccccc2)c1]

The 5-hydroxy-3-bromo-benzoic acid (1 g, 4.6 mmol), K₂CO₃ (1.33 g, 9.66 mmol), and BnBr (1.61 g, 9.43 mmol) were dissolved in dry DMF (10 mL) and stirred for 24 h. Water was added to the reaction mixture and extracted with EtOAc thrice (3×10 mL). The combined organic phase was evaporated and dissolved in 8N KOH in MeOH (50 mL) and refluxed for another 1 h. After completion of the reaction, the mixture was acidified with concentrated HCl to pH 2-3. The formed precipitate was filtered, dissolved in EtOAc and washed with water, brine and dried over Na₂SO₄. Removal of the solvent gave pure product as a light yellow color solid, m.p. 148-150° C. (1.1 g, 78% yield). ¹H NMR (400 MHz, d6-Acetone) δ: 11.8-10.8 (brs, 1H), 7.78, (s, 1H), 7.62, (s, 1H), 7.55-7.2 (m, 6H), 5.21 (s, 2H).

### 1,4-Dihydronaphth-2,3-diyl di-5-benzyloxy-3-bromobenzoate

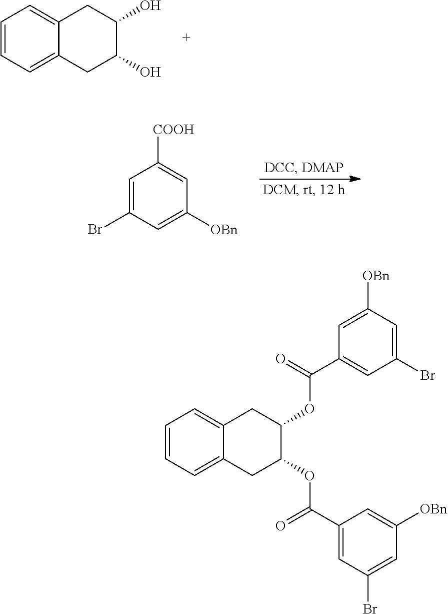 [SMILES: O[C@@H]1Cc2ccccc2C[C@@H]1O ; O=C(O)c1cc(Br)cc(OCc2ccccc2)c1 ; O=C(O[C@H]1Cc2ccccc2C[C@H]1OC(=O)c1cc(Br)cc(OCc2ccccc2)c1)c1cc(Br)cc(OCc2ccccc2)c1]

The diol 12 (50 mg, 0.3 mmol), 5-benzyloxy-3-bromo-benzoic acid (191 mg, 0.61 mmol), DCC (129 mg, 0.61 mmol) and DMAP (7 mg, 0.07 mmol) were dissolved in dry DCM (2 mL) and stirred at room temperature for 24 h. The DCM was removed and EtOAc (10 mL) was added and kept in the freezer for 12 h. The formed precipitate was filtered off, the solvent was removed and the crude product was purified by column chromatography using ethyl acetate and hexane as eluent. (1:3) to give the product as a white solid, m.p. 111-113° C. (145 mg, 65% yield). ¹H NMR (400 MHz, CDCl₃) δ: 7.68 (s, 2H), 7.48 (s, 2H), 4.4-7.18 (m, 16H), 5.69 (t, 2H), 4.95 (s, 2H), 3.32 (d, 4H).

### Compound 37

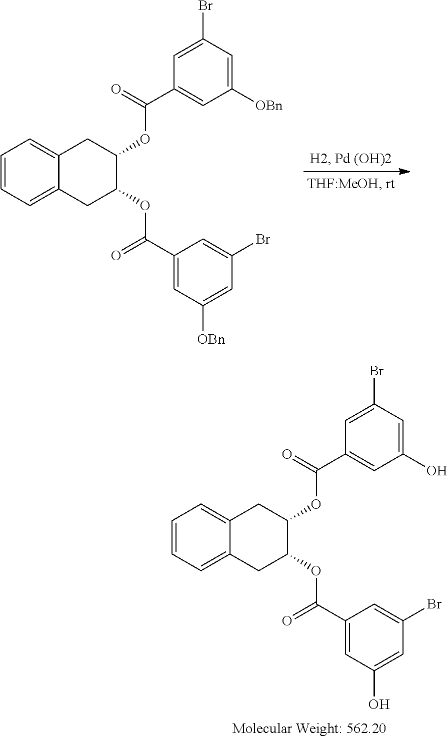 [SMILES: O=C(O[C@H]1Cc2ccccc2C[C@H]1OC(=O)c1cc(Br)cc(OCc2ccccc2)c1)c1cc(Br)cc(OCc2ccccc2)c1 ; O=C(O[C@H]1Cc2ccccc2C[C@H]1OC(=O)c1cc(O)cc(Br)c1)c1cc(O)cc(Br)c1]

The substrate (100 mg, 0.134 mmol), was dissolved in THF:MeOH (1:2) and Pd(OH)₂ (20 mg) was added and stirred at room temperature under H₂ atm for 3 h. After completion of the reaction as indicated by TLC, the reaction mixture was filtered through a small pad of celite and the solvents were removed under reduced pressure and purified by column chromatography to give compound 37 as a white solid, m.p. 78-80° C. (75 mg, 99% yield). ¹H NMR (400 MHz, d6-Acetone) δ: 8.68 (s, 2H) 7.46 (s, 3H), 7.31-7.19 (m, 5H), 7.06 (d, 2H), 5.72 (t, 2H), 3.41-3.32 (m, 4H).

### Compound 38

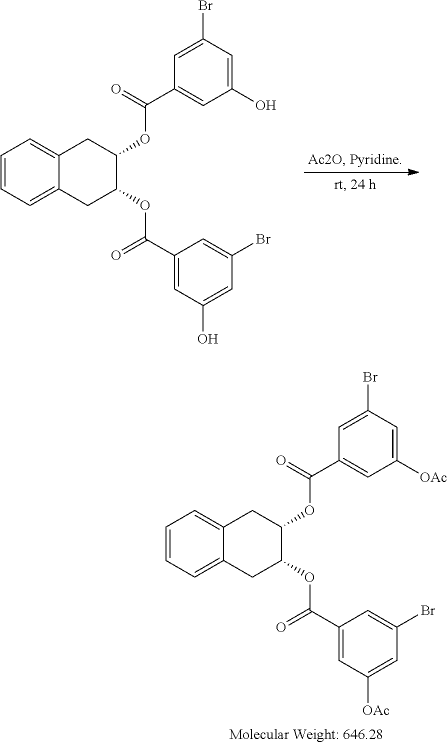 [SMILES: O=C(O[C@H]1Cc2ccccc2C[C@H]1OC(=O)c1cc(O)cc(Br)c1)c1cc(O)cc(Br)c1 ; CC(=O)Oc1cc(Br)cc(C(=O)O[C@H]2Cc3ccccc3C[C@H]2OC(=O)c2cc(Br)cc(OC(C)=O)c2)c1]

To the substrate (50 mg, 0.089 mmol) was added Ac₂O (0.5 mL), and pyridine (0.5 mL) and stirred for 24 h at room temperature. After completion of the reaction, the reaction mixture was diluted with ethyl acetate and washed with 1N HCl (10 mL), CuSO₄, (10 mL×3), brine (10 mL×3), dried over Na₂SO₄, and purified by column chromatography to give compound 38 as a white solid, m.p. 68-70° C. (51 mg, 87% yield). ¹H NMR (400 MHz, CDCl3) δ: 7.85 (s, 2H), 7.68 (s, 2H), 7.39 (t, 2H), 7.27-7.16 (m, 4H), 5.71 (t, 2H), 3.40-3.3 (m, 4H), 2.29 (s, 6H).

### Compound 36

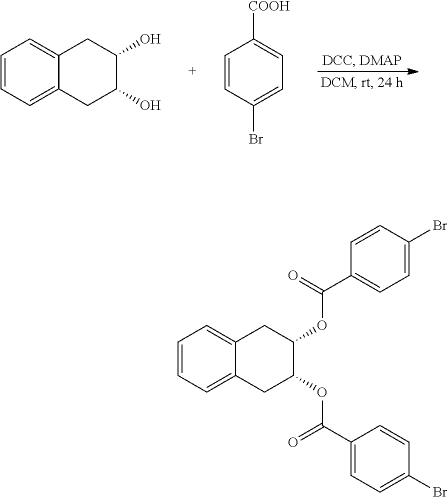 [SMILES: O[C@@H]1Cc2ccccc2C[C@@H]1O ; O=C(O)c1ccc(Br)cc1 ; O=C(O[C@H]1Cc2ccccc2C[C@H]1OC(=O)c1ccc(Br)cc1)c1ccc(Br)cc1]

The diol 12 (50 mg, 0.3 mmol), 4-bromobenzoic acid (125 mg, 0.61 mmol), DCC (128 mg, 0.61 mmol) and DMAP (7 mg, 0.07 mmol) were dissolved in dry DCM (2 mL) and stirred at room temperature for 24 h. The DCM was removed and EtOAc (10 mL) was added and kept in the freezer for 12 h. The formed precipitate was filtered off, The solvent was removed and the crude product was purified by column chromatography using ethyl acetate and hexane as eluent (1:4) to give compound 36 as a white solid, m.p. 160-162° C. (101 mg, 63% yield). ¹H NMR (400 MHz, CDCl₃) δ: 7.82 (d, 4H), 7.55 (d, 4H), 7.3-7.15 (m, 4H), 5.71 (t, 2H), 3.32 (d, 4H).

### Compound 35

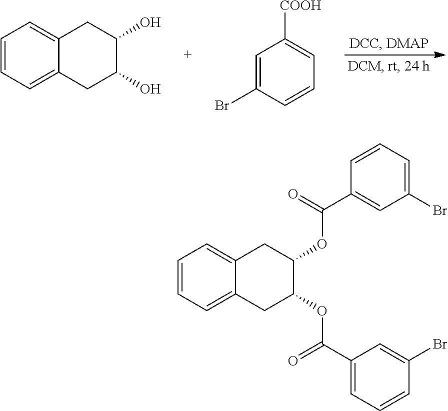 [SMILES: O[C@@H]1Cc2ccccc2C[C@@H]1O ; O=C(O)c1cccc(Br)c1 ; O=C(O[C@H]1Cc2ccccc2C[C@H]1OC(=O)c1cccc(Br)c1)c1cccc(Br)c1]

The diol 12 (50 mg, 0.3 mmol), 3-bromobenzoic acid (97 mg, 0.61 mmol), DCC (128 mg, 0.61 mmol) and DMAP (7 mg, 0.07 mmol) were dissolved in dry DCM (2 mL) and stirred at room temperature for 24 h. The DCM was removed and EtOAc (10 mL) was added and kept in the freezer for 12 h. The formed precipitate was filtered off. The solvent was removed and the crude product was purified by column chromatography using ethyl acetate and hexane as eluent (1:4) to give compound 35 as a white solid, m.p. 123-125° C. (100 mg, 61% yield). ¹H NMR (500 MHz, CDCl₃) δ: 8.1 (s, 2H), 7.89 (d, 2H), 7.65 (d, 2H), 7.3-7.18 (m, 6H), 5.74 (t, 2H), 3.34 (d, 4H).

### Inhibition of Purified 20S Proteasome Activity by EGCG and its Analogs.

A purified rabbit 20S proteasome (35 ng) was incubated with 20 μM of substrate Suc-LLVY-AMC in 100 μl assay buffer (20 mM Tris-HCl, pH 7.5), in the presence of EGCG or EGCG analogs at different concentrations or the solvent for 2 h at 37° C., followed by measurement of hydrolysis of the fluorogenic substrates using a Wallac Victor3™ multi-label counter with 355-nm excitation and 460-nm emission wavelengths.

### Inhibition of Cellular Proteasome by EGCG and its Analogs.

Human breast cancer MDA-MB-231 cells were treated with compound 5 or 7 for 24 hours. Cell lysates were subjected to chymotrypsin activity assay and Western blotting analysis as described before.

### MTT Assay.

Cells were grown in a 96-well plate. Triplicate wells of cells were treated with indicated concentrations of EGCG or EGCG analogs for 24 h. After aspiration of medium, MTT (1 mg/ml) was then added to the cell cultures, followed by incubation for 3 h at 37° C. After cells were crystallized, MTT was removed and DMSO was added to dissolve the metabolized MTT product. The absorbance was then measured on a Wallac Victor3 1420 Multi-label counter at 540 nm.

### Example 2

### Inhibition of Chymotrypsin-Like Activity of Purified 20S Proteasome

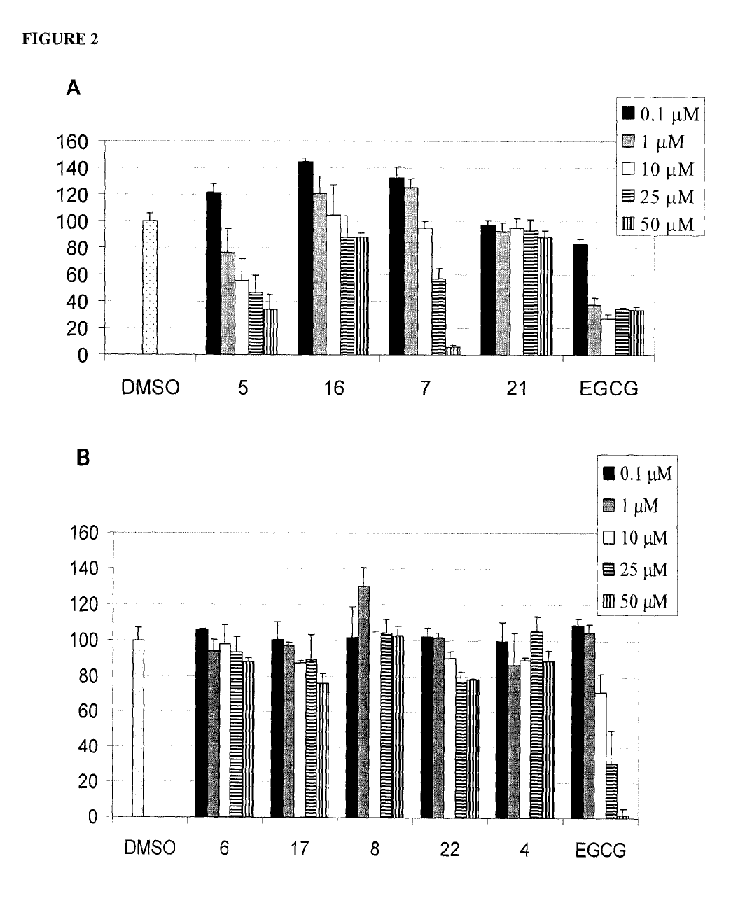

With reference to FIG. 2, EGCG potently inhibited the proteasomal chymotryptic activity consistent with our previous observation. Compound 5, which is a substituted tetralin that can be viewed as an analog of EGCG, inhibited the proteasomal chymotrypsin-like activity with an IC₅₀ value of 19 μM. Compound 16, which lacks a gallate moiety did not inhibit proteasomal chymotryptic activity even at a concentration of 50 μM. On the other hand, compound 7 (IC₅₀=29 μM) is only modestly less active than EGCG or 5 even though it lacks the gallate ester. Not surprisingly, compound 21 is not active in proteasome inhibition even at 50 μM. Upon acetylation none of the resulting derivatives 4, 6, 8, 17 and 22 exhibited proteasomal inhibition under these conditions.

### Example 3

### COMT Influences the Proteasome Inhibitory Activity of Derivatives 5 and 7

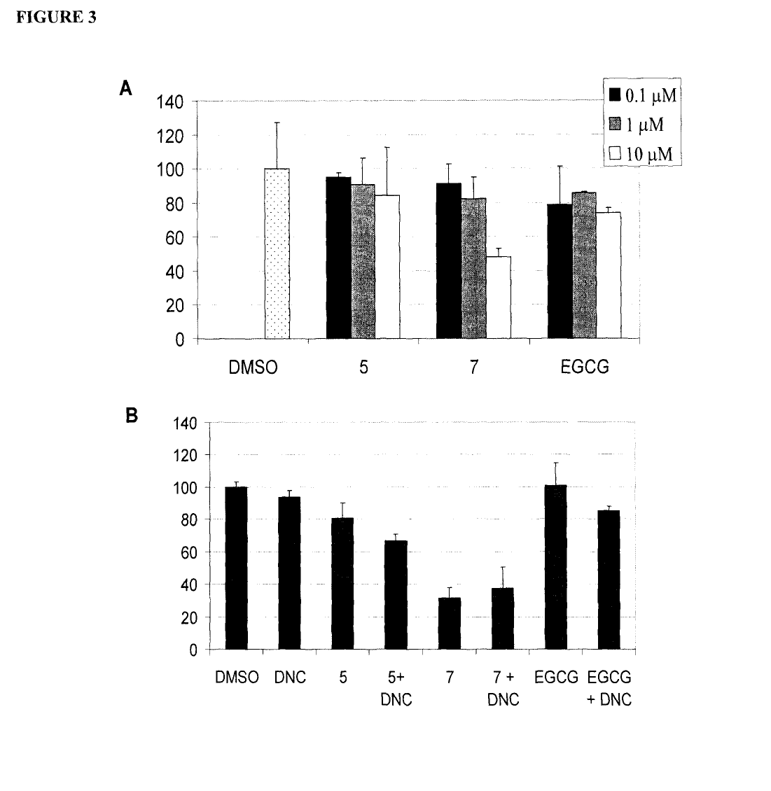

Human breast cancer MDA-MB-231 cell lysate that contains high COMT activity were treated with varying concentrations of compound 5 or 7. FIG. 3 illustrates that compound 7 at concentrations ranging between 1-10 μM inhibited proteasomal activity between 18-51% while compound 5 only inhibited proteasomal activity 10-16% under the same conditions. It would not have been expected from the data of EXAMPLE 2 that compound 7 is more active than compound 5 in inhibiting the proteasomal activity of MDA-MB-231 cell lysates. These results indicate that compound 5 may be more susceptible to biotransformation by COMT compared to compound 7. By comparison EGCG at 10 μM only inhibited the chymotrypsin-like activity in these cells by approximately 22%. Thus, consistent with previous reports EGCG is also susceptible to methylation by COMT (H., Lu, X. Meng, C. S. Yang, Drug Metabolism and Disposition; 31; 572, 2003).

### Example 4

### Inhibition of MDA-MB-231 Tumor Cell Growth

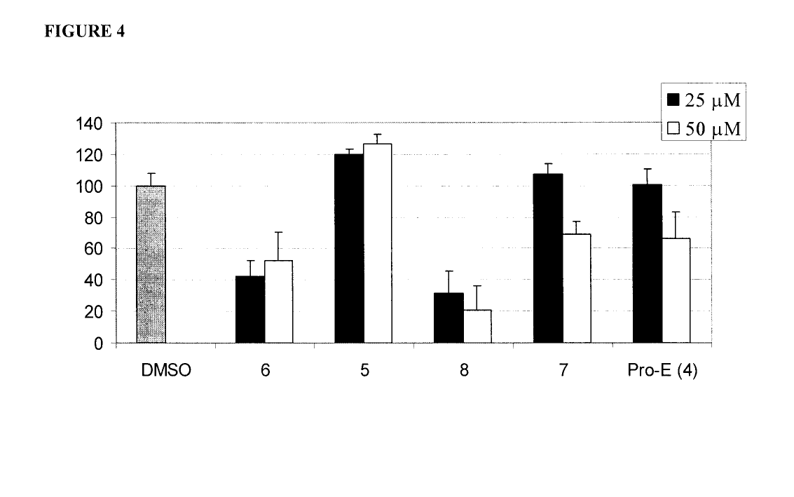

Compound 4 designated here as pro-EGCG exhibits enhanced growth inhibitory activity compared to EGCG (1) in a number of cancer cell lines (Lam, W. H. et al., Bioorg. Med. Chem. 2004, 12, 5587; Landis-Piwowar, K. R. et al., Internat. J. Mol. Med. 2005, 15 735). It has now been determined that the per-acetates 6 and 8 are more potent in inhibiting cell growth compared to their non-acetylated precursors 5 and 7. FIG. 4 shows the growth inhibitory activity of compound 4 compared to analogs 5 and 7 and their corresponding peracetates 6 and 8 in human breast cancer MDA-MB-231 cells. Surprisingly the per acetylated analog 8 was the most potent analog, exhibiting 70-79% inhibition in MDA-MB-231 cells growth at 25 to 50 μM. The per acetylated analog 6 induced about 50% inhibition in MDA-MB-231 cells Both per acetylated analogs were more potent than pro-EGCG 4 which showed 0 to 32% inhibition at equimolar concentrations.

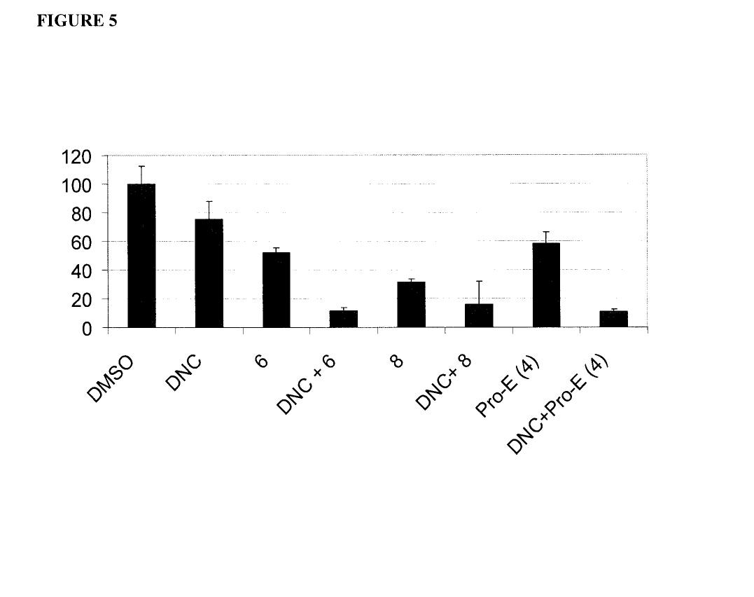

To determine whether the enhanced activity of the per acetylated analog 8 is due to diminished biotransformation, we examined whether the inhibitory activity of compounds 8 (the per-acetylated analog of 7) or 6 (the per-acetylated analog of 5) as well as that of pro-EGCG 4 is affected in the presence of 3,5-dinitrocatechol (DNC), a tight-binding inhibitor of COMT. If DNC inhibits COMT-mediated methylation of compound 5 or EGCG, one skilled in the art would observe increased growth-inhibitory activity of compound 6 or pro-EGCG on the addition of DNC. On the other hand, the growth inhibitory activity of compound 8 would not be significantly affected in the presence of DNC if analog 7 were not a substrate of COMT or would be less susceptible to its activity. MDA-MB-231 cells were treated with compounds 8 or 6, the per-acetylated analogs of compounds 7 or 5, in the presence or absence of DNC. Compound 6 alone at 50 μM inhibited cell proliferation by 48%. In the presence of 10 μM DNC, the inhibition of cell proliferation mediated by analog 6 increased to 88% (FIG. 5). A similar effect was observed with Pro-EGCG 4 whose inhibition of cell proliferations increased from 42% to 89% inhibition in the presence of DNC. In contrast to analog 6 and Pro-EGCG (4), the inhibition of cell proliferation mediated by analog 8 was not greatly enhanced in the presence of DNC (69% versus 84% inhibition) Thus the compound of the invention that lacks the chatechol unit on each of the adjacent aromatic rings is as susceptible to methylation mediated by COMT, which manifests higher inhibition of cell growth proliferation.

### Example 5

### Accumulation of Ubiquitinated Proteins

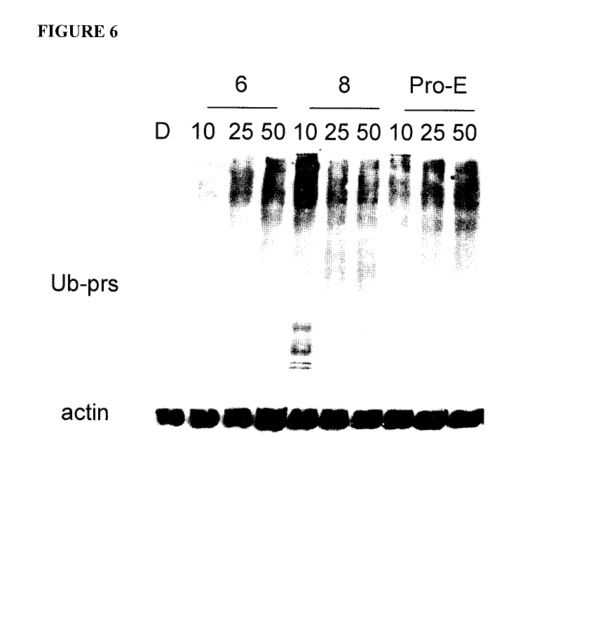

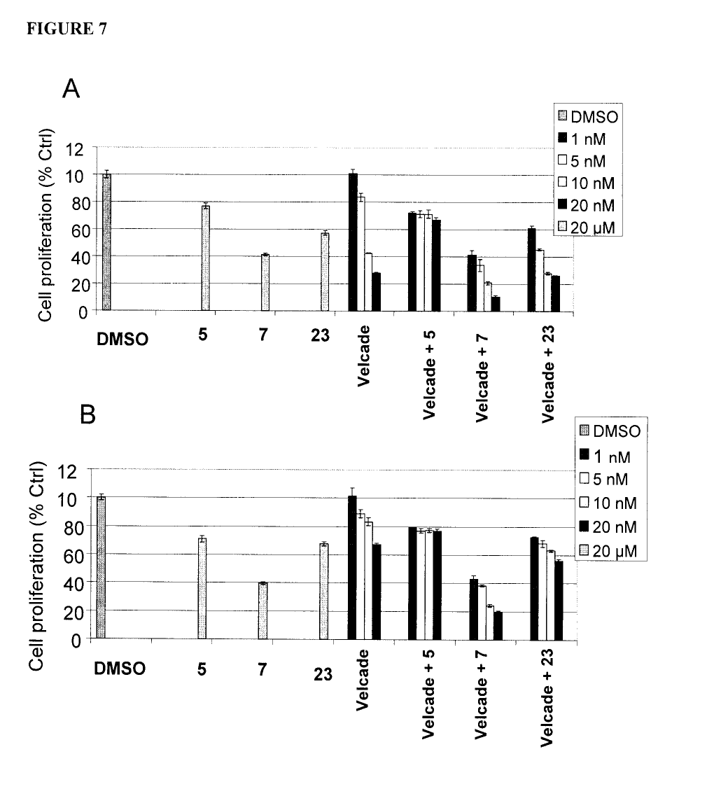

With reference to FIG. 6, the experiments of this example 5 were undertaken to investigate whether in MDA-MB-231 cells pre-acetylated compounds 6 and compound 8 as well as Pro-EGCG 4 would be able to inhibit proteasome and manifest accumulation of ubiquitinated proteins. Indeed, at the doses tested and illustrated in FIG. 7, higher levels of ubiquitinated proteins were accumulated by compound 8 compared to compound 6 and Pro-EGCG 4 in MDA-MB-231 cells, indicating that more proteasome activity was inhibited by analog 8.

### Example 6

### Inhibition of Cell Proliferation of Human Multiple Myeloma Cells by Compounds of the Invention in Combination with Bortezomib (Velcade™)

When combined with Velcade™, compounds 7 and 23 but not 5 showed a synergistic inhibitory effect against cell proliferation in human multiple myeloma cells. Among the three analogs, compound 7 was the most potent inhibitor of cell proliferation. Cell proliferation was inhibited approximately 60% in ARP cells treated with 20 μM of compound 7 (FIG. 2A). Treatment with Velcade™ reduced cell proliferation in a dose-dependent manner and further inhibition was observed when combined with compound 7 (FIG. 7A). The inhibitory effect of bortezomib was interfered with by co-treatment with compound 5 antagonized the inhibitory effect of Velcade™ (FIG. 7A). The compound 23 also increased Velcade™ bortezomib-induced inhibition of cell proliferation but the effect was weaker than that seen with compound 7 (FIG. 7A).

The data generated from OPM1 cells showed a similar pattern of effects. However the OPM1 cell line appears to be more resistant to treatment with Velcade™ bortezomib and the various combinations (FIG. 7B).

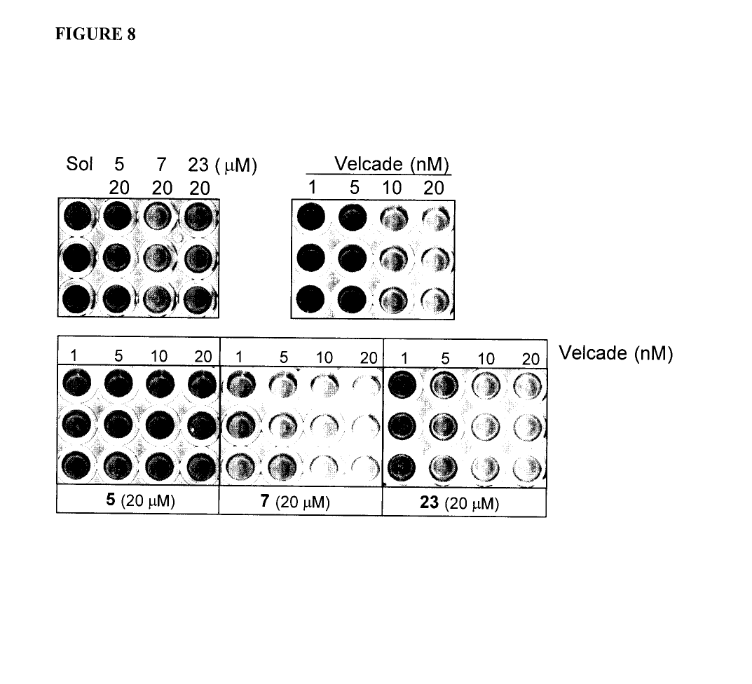

In FIG. 8, the color changes of the MTT assay in a 96 well-plate (in the same experiment shown in FIG. 7A) using ARP cells are presented. Deep purple color indicates fully viable cells; light purple color indicates a reduced number of viable cells; and yellowish color indicates an absence of viable cells (FIG. 8). The color change pattern was consistent with the potencies of the compounds for inhibiting ARP tumor cell growth (compare FIGS. 8 and 7A). The results depicted given in FIGS. 7 and 8 show that compounds 7 and 23 unexpectedly exhibit synergistic effect on human multiple myeloma cells when combined with Velcade™ bortezomib, while and compound 5 could partially block the inhibitory effect of Velcade™ bortezomib on these malignant cells.

### Example 7

### EGCG Analogs Pro-EGCG can Activate AMPK Signaling

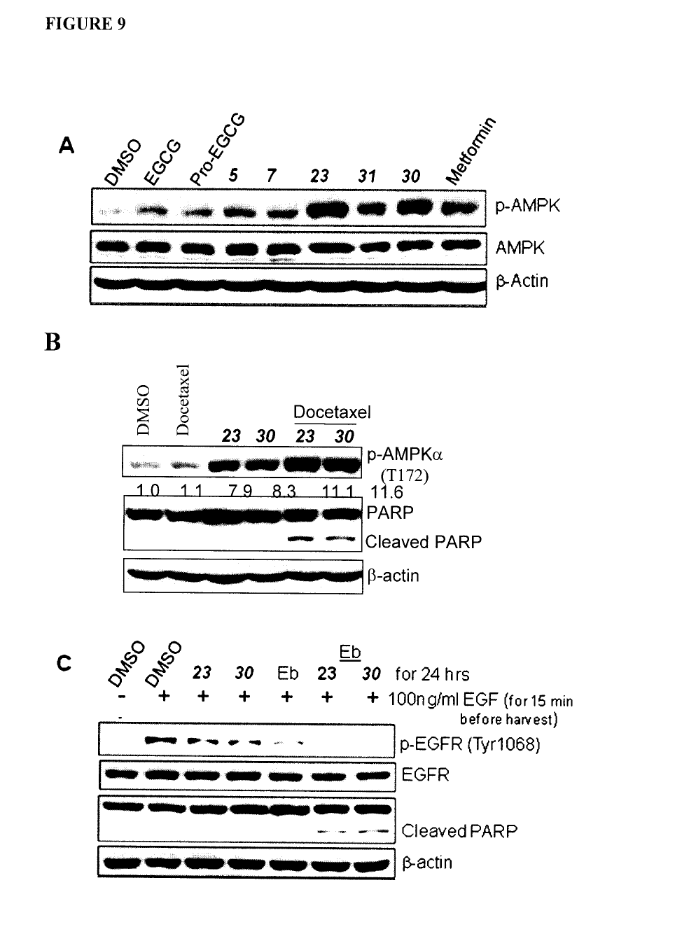

Activation of AMP-activated protein kinase (AMPK), a physiological cellular energy sensor, strongly suppresses cell proliferation, induces apoptosis and reduces the stem cell population in cancer cells. Many studies have reported that metformin, an anti-diabetes drug, is a potent AMPK activator in different cancer cells. AMPK activators have the potential to inhibit tumor cell growth and produce synergistic effects when combined with anti-cancer drugs. We tested several natural and synthetic compounds in human breast cancer cells and found that EGCG (a green tea polyphenol) and Pro-EGCG (a synthetic EGCG analog; compound 4) can activate AMPK signaling (FIG. 9A).

In our studies, the human breast cancer cell line and treatments of the cells in vitro were as follows: The human breast cancer cell line MDA-MB-231 derives from a human adenocarcinoma that metastatizes to the pleural effusion (Cailleau et al., In Vitro, 1978, 14: 911-915; Cailleau et al., Journal of the National Cancer Institute, 1974, 53: 661-674). This cell line expresses high levels of EGFR (Godden et al., Anticancer Res., 1992, 12: 1683-1688), and is one of the breast cancer cell lines for the study of hormone-independent and triple negative breast cancer. The MDA-MB-231 cells were obtained from American Type Culture Collection (Manassas, Va.) and grown in D-MEM/F-12 medium supplemented with 10% FBS and were maintained at 37° C. and 5% CO₂ (Chen et al., Cancer research, 2006, 66: 10425-10433). In our studies, the MDA-MB-231 cells were treated with EGCG, EGCG analogs or combination treatment with anti-cancer drugs Docetaxel or Erlotinib. Metformin, an anti-diabetes drug and AMPK activator, was used as a positive control.

The Western blot analysis was performed as follows: A whole cell extract was prepared from the treated cells as described previously (Landis-Piwowar et al., Cancer research, 2007, 67: 4303-4310; Chen et al., Cancer research, 2007, 67: 1636-1644; Chen et al., Biochemical pharmacology, 2005, 69: 1421-1432). The cell extracts (30 μg) were then separated by an SDS-PAGE gel and transferred to nitrocellulose membranes. The membrances were blotted by specific antibodies including anti-AMPK, p-AMPK, EGFR, p-EGFR (Cell Signaling Tech. Danvers, Mass.), PARP (Enzo Life Sciences, Plymouth Meeting, Pa.), actin (Santa Cruz Biotechnology, Santa Cruz, Calif.). The membranes were visualized by enhanced chemiluminescence, as described previously (Landis-Piwowar et al., Cancer research, 2007, 67: 4303-4310; Chen et al., Cancer research, 2007, 67: 1636-1644; Chen et al., Biochemical pharmacology, 2005, 69: 1421-1432).

The flow cytometry analysis was performed as follows: The treated cells were washed once with phosphate-buffered saline (PBS) and then harvested with Cell Dissociation Buffer (enzyme-free, Invitrogen, cat. #13150-016). Detached cells were washed with PBS containing 1% FCS (a wash buffer), and resuspended in the wash buffer (1,000 cells/100 μl). The cell were stained with combinations of fluorochrome-conjugated monoclonal antibodies obtained from BD Biosciences (San Diego, Calif., USA) against human CD44 (FITC; cat. #555478) and CD24 (PE; cat. #555428) or their respective isotype controls at concentrations recommended by the manufacturer and incubated at 4° C. in the dark for 30 to 40 min. The labeled cells were washed in the wash buffer, then fixed in PBS containing 1% paraformaldehyde, and then analyzed on a FACSVantage (BD Biosciences) (Sheridan et al., Breast Cancer Res., 2006, 8: R59).

In FIG. 9A, human breast cancer MDA-MB-231 cells were treated with 20 μM of EGCG, Pro-EGCG and other EGCG analogs (compounds 5, 7, 23, 30 and 31), or 10 mM of metformin for 3 hrs. Cell lysates were analyzed by Western blot using antibodies of anti-AMPK, p-AMPK, PARP, p-EGFR, EGFR or β-actin. In FIG. 9B, human breast cancer MDA-MB-231 cells were treated with 20 μM of EGCG analogs 23 and 30, 10 nM of docetaxel alone, or combined treatment with compounds 23 and 30 plus docetaxel for 24 hrs.

In order to discover more AMPK activators, we tested a series of EGCG analogs in human breast cancer cells. The MDA-MB-231 cells were treated with 20 μM of EGCG analogs 5, 7, 23, 30 and 31. EGCG, Pro-EGCG and Metformin were used as positive controls. We found that EGCG analogs 23 and 30 were more potent AMPK activators even at lower concentration than metformin (FIG. 9A). EGCG analogs 23 and 30 could also sensitize these TNBC cells to Docetaxel, and the combination treatment induced more apoptotic(as reflected by increased PARP cleavage) cell death than each treatment alone (FIG. 9B). EGCG analogs 23 and 30 could also sensitize these TNBC cells to the EGFR inhibitor Erlotinib and the combination treatment was more effective than each treatment alone in terms of reducing p-EGFR and inducing apoptotic cell death(as reflected by increased PARP cleavage) (FIG. 9C).

The results show that EGCG analogs are potent AMPK activators in breast cancer cells. Indeed, the EGCG analogs 23 and 30 were more potent AMPK activators than EGCG and Pro-EGCG, and even more potent than metformin (FIG. 9A). In addition, synergistic effects were found when these EGCG analogs were used in combination with other anti-cancer drugs such as docetaxel and erlotinib.

### Example 8

### EGCG Analogs 23 and 30 Significantly Decreased a Population of CD44^{high}/CD24^{low} Cells in TNBC Cells

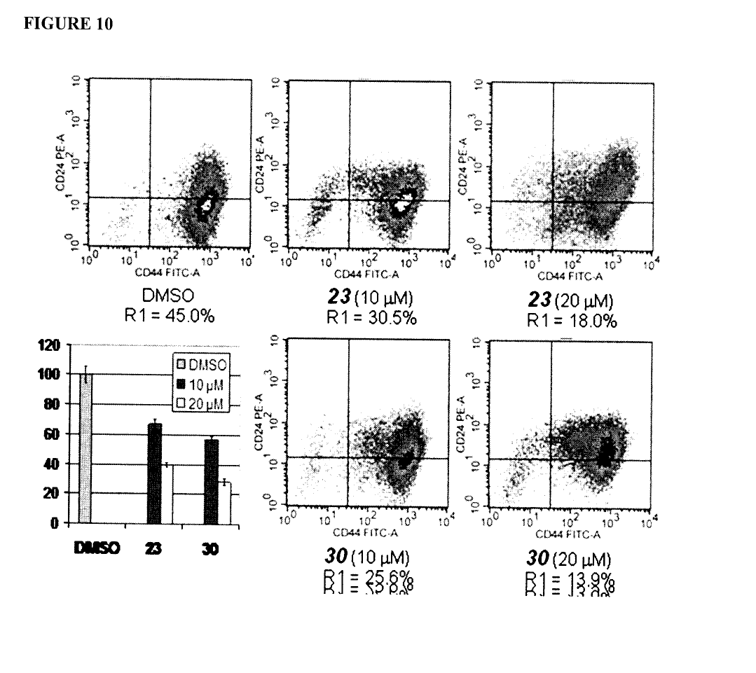

Metformin could selectively target cancer stem cells and reduce the CD44high/CD24low cell population in TNBC cells through activation of AMPK signaling (Hirsch et al., Cancer Research, 2009, 69: 7507-7511). To determine whether EGCG analogs 23 and 30 can reduce the stem cell population in the breast cancer cells, the human breast cancer MDA-MB-231 cells were treated with different concentrations of compounds 23 and 30 for 48 hours. The treated cells were stained with special antibodies against human CD44 (FITC), CD24 (PE) or their respective isotype controls, followed by washing, fixing and analysed by flow cytometry. Treatment of the MDA-MB-231 cells with 10 or 20 μM of compound 30 resulted in a 43.3% and 71.7% decrease of the CD44high/CD24low population, respectively (FIG. 10).

The results show that both EGCG analogs 23 and 30 can reduce the CD44high/CD24low cell population, and compound 30 was more potent than compound 23.

In summary, the results show that EGCG analogs 23 and 30 can activate AMPK and can enhance the efficacy of clinical anticancer drugs. In the above experiments, MDA-MB-231 cells were combinationally treated with 23 or 30 plus Docetaxel, and treated with 23, 30 or Docetaxel alone as controls. The results showed that only the combination treatment induced apoptotic cell death at the treatment condition (FIG. 9B). The results suggest that EGCG analogs can sensitize TNBC cells to EGFR inhibitors. We tested this hypothesis in the same cell line and demonstrated that EGCG analogs 23 and 30 showed synergistic effect when combined with an EGFR inhibitor Erlotinib (FIG. 9C). Interestingly, EGCG analogs 23 and 30 can reduce CD44^{high}/CD24^{low} cell population in TNBC cells, probably associated with their AMPK activation property (FIG. 10).

### Example 9

### EGCG Analogs 23 and 30 Significantly Inhibited Mammosphere Formation

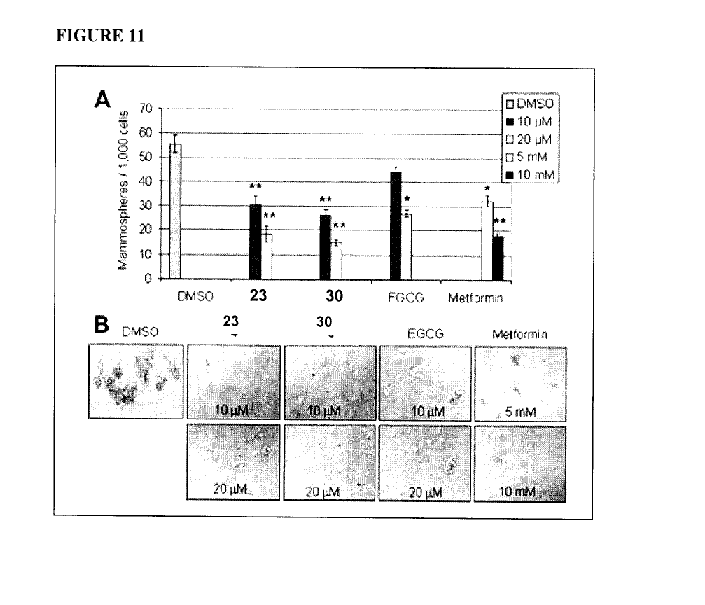

Tumor stem cells have the characteristic of forming tumor spheres. An experiment of mammosphere formation is a useful tool to identify a human mammary stem/progenitor-cell population and measure stem cell-like behavior. To examine whether compounds 23 and 30 can target cancer stem or stem-like cells and inhibit mammosphere formation, we conducted a mammosphere formation assay. Metformin and EGCG were used as controls. The results showed that treatment of MDA-MB-231 cells with 10 or 20 μM of 23 or 30 for 7 days resulted in inhibition of mammosphere formation by 45.1% and 66.7% or 52.2% and 73.3%, respectively (FIG. 11). As comparisons, treatment with 10 or 20 μM of EGCG only inhibited mammosphere formation by 20.1% and 51.3%, and treatment with 5 and 10 mM of metformin inhibited mammosphere formation by 41.4% and 67.6%, respectively (FIG. 11). Therefore, EGCG analogs 23 and 30 were much more potent than EGCG and metformin in terms of inhibition of mammosphere formation.

### Mammosphere Formation Assay

Mammosphere formation assay was performed to assess the capacity of cancer stem cell self-renewal. Single cell suspensions of MDA-MB-231 cells were thoroughly suspended and plated on ultra low adherent wells of 6-well plates (Corning, Lowell, Mass.) at 1000 cells/well in 1.5 ml of sphere formation medium (1:1 DMEM/F12 medium supplemented with 50 units/ml penicillin, 50 mg/ml streptomycin, B-27 and N-2). One milliliter of sphere formation medium was added every 3-4 days. After 7 days of incubation with different concentrations of EGCG analogs 23, 30 or metformin, the formed spheres were collected by centrifugation at 300 g for 5 min and counted with an inverted phase-contrast Zeiss Axiovert 25 microscope.

### Example 10

### EGCG Analogs 23 and 30 Inhibited Proliferation of Breast Cancer Cells in a Dose-Dependent Manner Associated with Activation of AMPK and Induction of p21 Protein

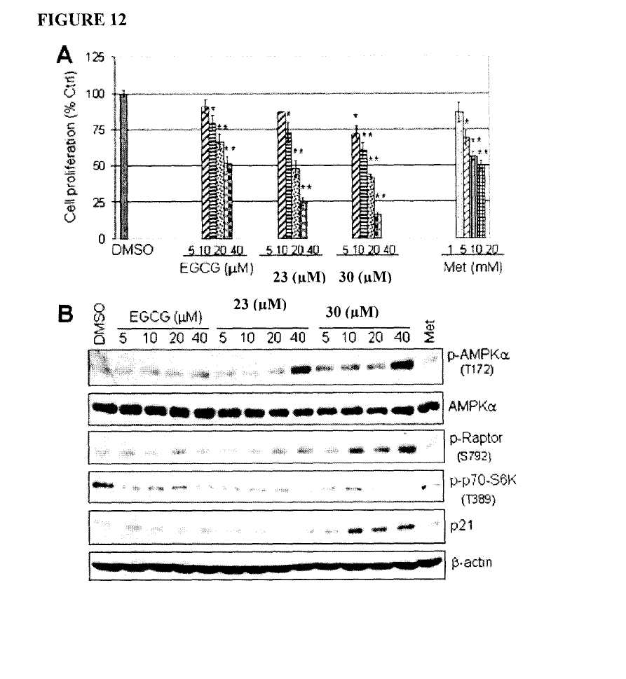

It has been reported that AMPK activation by an authentic AMPK activator AMP-mimetic 5-aminoimidazole-4-carboxamide ribonucleoside (AICAR) results in cell cycle arrest and inhibition of cell proliferation in hepatoma HepG2 cells. In order to determine whether EGCG analogs 23 and 30 could play a similar role as AI-CAR in suppression of cell proliferation, we treated human breast cancer MDA-MB-231 cells with different concentrations of compounds 23 and 30, followed by Western-blot analysis and cell proliferation assay. Cells treated with metformin and natural product of EGCG were used as controls. The results showed that both EGCG analogs 23 and 30 could inhibit cell proliferation in a dose-dependent manner and their inhibitory effects were more potent than EGCG and metformin even when analogs 23 and 30 were used at much lower concentrations compared with metformin treatment (FIG. 12A). Results in Western blot showed that compounds 23 and 30 activated AMPK in a dose-dependent manner as well, as measured by increased levels of phosphor-AMPKα and phosphor-Raptor, one of the direct downstream substrate proteins of AMPK (FIG. 12B). Our data also showed that activation of AMPK by 23 and 30 could suppress mTOR pathway measured by decreased phosphor-p70-S6K (FIG. 12B), demonstrating the functionality of these EGCG analogs as AMPK activators. Inhibition of breast cancer cell proliferation by treatment with compounds 23 and 30 was associated with increased levels of p21 protein (FIG. 12B).

While specific embodiments of the present invention have been described in the examples, it is apparent that modifications and adaptations of the present invention will occur to those skilled in the art. The embodiments of the present invention are not intended to be restricted by the examples. It is to be expressly understood that such modifications and adaptations which will occur to those skilled in the art are within the scope of the present invention, as set forth in the following claims. For instance, features illustrated or described as part of one embodiment can be used in another embodiment, to yield a still further embodiment. Thus, it is intended that the present invention cover such modifications and variations as come within the scope of the claims and their equivalents.

The contents of all documents and references cited herein are hereby incorporated by reference in their entirety.

1.-121. (canceled)

122. A compound having the structure of formula I: 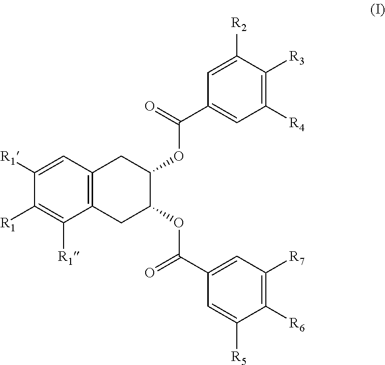
wherein:
R₁, R₁′ and R₁″ are each independently selected from the group consisting of H, alkyl, alkenyl, cycloalkyl, heterocycloalkyl, cycloalkenyl, heterocycloalkenyl, aryl, halogen, OH, an acyloxy group, and NR₈, R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, alkyl, OH, acyloxy, NR₈R₉ or a halogen, wherein R₈ and R₉ are as defined above;
or an analog or a pharmaceutically acceptable salt thereof;
with the proviso that when R₁, R₁′ and R₁″ are all H and R₂, R₄, R₅ and R₇ are all OH, then R₃ and R₆ are not H or OH; and when R₁, R₁′ and R₁″ are all H and R₂, R₄, R₅ and R₇ are all acyloxy, then R₃ and R₆ are not H or acyloxy.

123. The compound of claim 122, wherein the compound has the structure of formula XI: 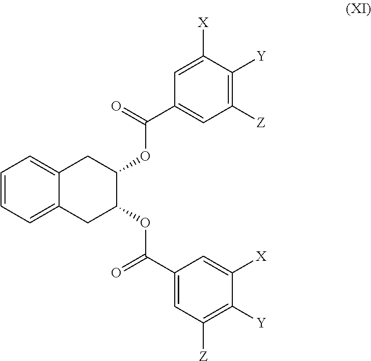
wherein:
X, Y and Z are each independently H, Br, F, Cl, OH, Me, NH₂, OAc, NHAc or CF₃; or an analog or a pharmaceutically acceptable salt thereof;
with the proviso that, when X and Z are both OH, then Y is not H or OH; and when X and Z are both OAc, then Y is not H or OAc.

124. The compound of claim 123, wherein X and Z are the same.

125. A pharmaceutical composition comprising a compound of claim 122 and a pharmaceutically acceptable carrier.

126. A method for activating AMPK in a cell, comprising contacting the cell with an effective amount of at least one compound having the structure of formula I: 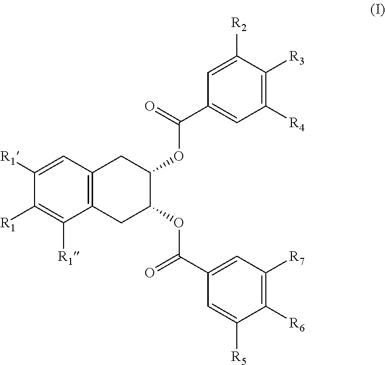
wherein:
R₁, R₁′ and R₁″ are each independently selected from the group consisting of H, alkyl, alkenyl, cycloalkyl, heterocycloalkyl, cycloalkenyl, heterocycloalkenyl, aryl, halogen, OH, an acyloxy group, and NR₈, R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, alkyl, OH, acyloxy, NR₈R₉ or a halogen, wherein R₈ and R₉ are as defined above;
or an analog or pharmaceutically acceptable salt thereof;
such that AMPK activity in the cell is activated.

127. The method of claim 126, wherein said contacting occurs in vivo.

128. The method of claim 127, wherein said contacting comprises administering the at least one compound to a subject by a route selected from the group consisting of oral, parenteral, subcutaneous, intravenous, intramuscular, intraperitoneal, intraarterial, transdermal, and mucosal administration.

129. A method for inhibiting tumor cell growth and/or treating cancer in a subject, comprising administering to the subject a therapeutically effective amount of at least one compound of claim 126, or an analog or pharmaceutically acceptable salt thereof, such that tumor cell growth is inhibited and/or cancer is treated in the subject.

130. The method claim 129, wherein cancer stem cell population, activity of epidermal growth factor receptor (EGFR), or NF-kB, PI3K/Akt and/or mTOR signaling pathways are decreased or inhibited.

131. The method of claim 129, wherein the CD44^{high}/CD24^{low} cell population is reduced.

132. The method of claim 129, wherein the compound or composition is administered orally, parenterally, subcutaneously, intravenously, intramuscularly, intraperitoneally, intraarterially, transdermally, or through the mucosa.

133. The method of claim 129, wherein the subject is a human.

134. The method of claim 129, wherein the compound or composition is administered in combination with a second therapeutic agent.

135. The method of claim 134, wherein the second therapeutic agent is an anti-cancer therapeutic agent, a chemotherapeutic agent, an EGFR inhibitor, an AMPK activator and/or a proteasomal inhibitor.

136. A method for treating a metabolic disorder, comprising administering to a subject in need thereof a therapeutically effective amount of at least one compound of claim 126, or an analog or pharmaceutically acceptable salt thereof, such that the metabolic disorder is treated.

137. A method for increasing the response of a disease to a proteasome inhibitor, comprising administering a therapeutically effective amount of at least one compound of claim 126 and the proteasome inhibitor to a subject in need thereof.

138. A method for activating AMPK in a cell, comprising contacting the cell with an effective amount of at least one compound having the structure of formula XI: 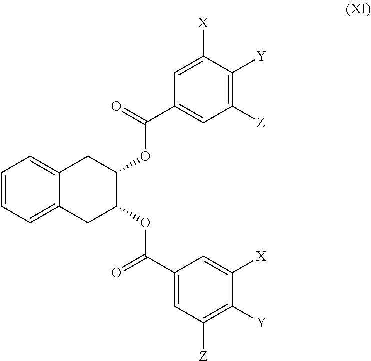
wherein:
X, Y and Z are each independently H, Br, F, Cl, OH, Me, NH₂, OAc, NHAc or CF₃; or an analog or pharmaceutically acceptable salt thereof;
such that AMPK activity in the cell is activated.

139. A method for inhibiting tumor cell growth and/or treating cancer in a subject, comprising administering to the subject a therapeutically effective amount of at least one compound of claim 138, or an analog or pharmaceutically acceptable salt thereof, such that tumor cell growth is inhibited and/or cancer is treated in the subject.

140. The method of claim 139, wherein the tumor or cancer is triple-negative breast cancer (TNBC) or is hormone-dependent.

141. A method for treating a metabolic disorder, comprising administering to a subject in need thereof a therapeutically effective amount of a compound of claim 138, or an analog or pharmaceutically acceptable salt thereof, such that the metabolic disorder is treated.
